(19) United States
(12) Patent Application Publication
(10) Pub. No.: US 2026/0164920 A1
(43) Pub. Date: Jun. 11, 2026

(54) COMPOUND, COMPOSITION, ORGANIC OPTOELECTRONIC DEVICE, AND DISPLAY DEVICE

(71) Applicant: SAMSUNG SDI CO., LTD., Yongin-si (KR)

(72) Inventors: Dosik KIM, Suwon-si (KR); Eunhye AN, Suwon-si (KR); Ho Kuk JUNG, Suwon-si (KR); Dongjin JANG, Suwon-si (KR); Ji Hun SHIN, Suwon-si (KR); Soonhyung KWON, Suwon-si (KR); Seokwoo KANG, Suwon-si (KR)

(21) Appl. No.: 19/402,035

(22) Filed: Nov. 26, 2025

(30) Foreign Application Priority Data
Dec. 10, 2024 (KR) 10-2024-0182811

(51) Int. Cl. H10K 50/12; C07D 403/14; H10K 85/30; H10K 85/40; H10K 85/60

(52) U.S. Cl. CPC H10K 50/12; C07D 403/14; H10K 85/342; H10K 85/346; H10K 85/40; H10K 85/622; H10K 85/626; H10K 85/654; H10K 85/6572; H10K 85/6574; H10K 85/6576; H10K 85/658

(57) ABSTRACT

A compound, a composition including the compound, an organic optoelectronic device including the compound or composition, and a display device including the organic optoelectronic device, the compound being represented by Chemical Formula 1:

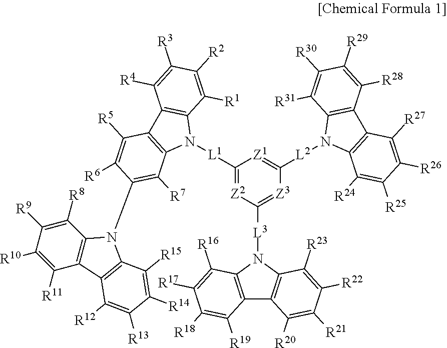

### CROSS-REFERENCE TO RELATED APPLICATION

This application claims priority to and the benefit of Korean Patent Application No. 10-2024-0182811 filed with the Korean Intellectual Property Office on Dec. 10, 2024, the entire contents of which are incorporated herein by reference.

### BACKGROUND

### 1. Field

Embodiments relate to a compound, a composition, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively, and the other is a light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

The embodiments may be realized by providing a compound, the compound being represented by Chemical Formula 1:

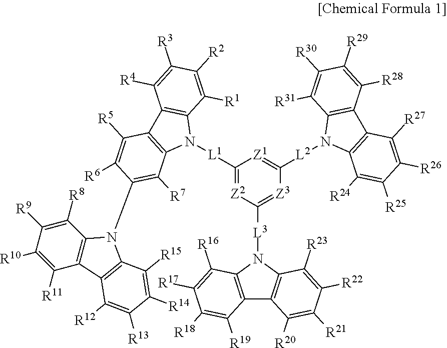

wherein, in Chemical Formula 1, Z¹ to Z³ are each independently N or CR^{a}, provided that at least two of Z¹ to Z³ are N, L¹ to L³ are each independently a single bond or a substituted or unsubstituted C6 to C20 arylene group, provided that one or two of L¹ to L³ are a substituted or unsubstituted o-phenylene group or a substituted or unsubstituted m-phenylene group, and R¹ to R³¹ and R^{a} are each independently hydrogen, deuterium, a substituted or unsubstituted C1 to C30 alkyl group, a substituted or unsubstituted C6 to C30 aryl group, a substituted or unsubstituted C2 to C30 heterocyclic group, excluding a carbazolyl group, a substituted or unsubstituted silyl group, a substituted or unsubstituted amine group, a halogen, a cyano group, or a combination thereof.

The embodiments may be realized by providing a composition, the composition including a first compound, the first compound being the compound according to an embodiment, and a second compound, the second compound being different from the first compound and including at least one carbazole moiety.

The embodiments may be realized by providing an organic optoelectronic device including an anode and a cathode facing each other, and an organic layer between the anode and the cathode, the organic layer including the compound according to an embodiment.

The embodiments may be realized by providing an organic optoelectronic device including an anode and a cathode facing each other, and an organic layer between the anode and the cathode, the organic layer including the composition according to an embodiment.

The embodiments may be realized by providing a display device including the organic optoelectronic device according to an embodiment.

### BRIEF DESCRIPTION OF THE DRAWINGS

Features will become apparent to those of skill in the art by describing in detail exemplary embodiments with reference to the attached drawings in which:

FIG. 1 is a cross-sectional view showing an example of an organic light emitting diode, which is an example of an organic optoelectronic device according to some example embodiments, and

FIG. 2 is a cross-sectional view showing another example of an organic light emitting diode, which is an example of an organic optoelectronic device according to some example embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, or one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a halogen, or a cyano group. In a specific example of the present embodiments, the “substituted” refers to replacement of at least one hydrogen of a substituent or a compound by deuterium, a C1 to C20 alkyl group, a C6 to C30 aryl group, or a cyano group. In a specific example of the present embodiments, the “substituted” refers to replacement of at least one hydrogen of a substituent or a compound by deuterium, a C1 to C5 alkyl group, a C6 to C18 aryl group, or a cyano group. In a specific example of the present embodiments, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of any hydrogen atom by another substituent and thus the remaining of all of the hydrogen atoms.

In the present specification, “hydrogen (—H)” may also include “deuterium substitution (—D)” or “tritium substitution (—T).” For example, any hydrogen in any compound described herein may be protium, deuterium, or tritium (e.g., based on natural or artificial substitution).

As used herein, the term “or” is not an exclusive term, e.g., “A or B” would include A, B, or A and B.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

As an example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, or a combination thereof.

As used herein, hole characteristics refer to an ability to donate an electron to form a hole when an electric field is applied. A hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied. Electrons formed in the cathode may be easily injected into the light emitting layer and transported in the light emitting layer due to conductive characteristics according to a lowest unoccupied molecular orbital (LUMO) level.

Hereinafter, a compound according to example embodiments is described.

The compound according to example embodiments may be represented by Chemical Formula 1.

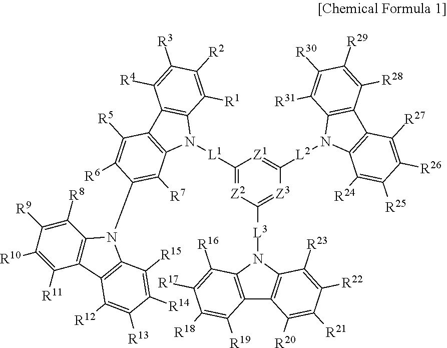

In Chemical Formula 1, Z¹ to Z³ may each independently be or include, e.g., N or CR^{a}, provided at least two of Z¹ to Z³ are N.

L¹ to L³ may each independently be or include, e.g., a single bond or a substituted or unsubstituted C6 to C20 arylene group, provided that one or two of L¹ to L³ are a substituted or unsubstituted o-phenylene group or a substituted or unsubstituted m-phenylene group.

R¹ to R³¹ and R^{a} are each independently hydrogen, deuterium, a substituted or unsubstituted C1 to C30 alkyl group, a substituted or unsubstituted C6 to C30 aryl group, a substituted or unsubstituted C2 to C30 heterocyclic group, excluding a carbazolyl group, a substituted or unsubstituted silyl group, a substituted or unsubstituted amine group, a halogen, a cyano group, or a combination thereof.

The compound represented by Chemical Formula 1 may effectively separate HOMO/LUMO distributions by asymmetrically arranging substituted or unsubstituted carbazolyl groups and substituted or unsubstituted biscarbazolyl groups having hole properties centered on a nitrogen-containing ring having electronic properties, and thus, if the compound is applied to a device, life-span characteristics of the device may be improved. In addition, the compound represented by Chemical Formula 1 may help lower an operating voltage and increase luminous efficiency by specifying the bonding position of the substituted or unsubstituted biscarbazolyl group.

In an implementation, in Chemical Formula 1, Z¹ to Z³ may each be N.

In an implementation, in Chemical Formula 1, Z¹ and Z² may each be N and Z³ may be CR^{a}.

In an implementation, in Chemical Formula 1, Z¹ and Z³ may each be N and Z² may be CR^{a}.

In an implementation, in Chemical Formula 1, Z² and Z³ may each be N and Z¹ may be CR^{a}.

In an implementation, in Chemical Formula 1, L¹ to L³ may each independently be, e.g., a single bond, a substituted or unsubstituted o-phenylene group, a substituted or unsubstituted m-phenylene group, a substituted or unsubstituted p-phenylene group, a substituted or unsubstituted biphenylene group, a substituted or unsubstituted terphenylene group, a substituted or unsubstituted naphthylene group, a substituted or unsubstituted benzofuranylene group, a substituted or unsubstituted dibenzofuranylene group, a substituted or unsubstituted benzothiophenylene group, a substituted or unsubstituted dibenzothiophenylene group, or a substituted or unsubstituted fluorenylene group, provided that one or two of L¹ to L³ are a substituted or unsubstituted o-phenylene group or a substituted or unsubstituted m-phenylene group.

In an implementation, in Chemical Formula 1, one or two of L¹ to L³ may be a substituted or unsubstituted o-phenylene group.

In an implementation, in Chemical Formula 1, at least one of L¹ to L³ may be different from the others, and thus the compound may have an asymmetric structure.

In an implementation, in Chemical Formula 1, one of L¹ to L³ may be a substituted or unsubstituted o-phenylene group and two of L¹ to L³ may be single bonds.

In an implementation, in Chemical Formula 1, two of L¹ to L³ may be substituted or unsubstituted o-phenylene groups and one of L¹ to L³ may be a single bond.

In an implementation, in Chemical Formula 1, one of L¹ to L³ may be a substituted or unsubstituted o-phenylene group, one of L¹ to L³ may be a substituted or unsubstituted m-phenylene group, and one of L¹ to L³ may be a single bond.

In an implementation, in Chemical Formula 1, R¹ to R³¹ and R^{a} may each independently be, e.g., hydrogen, deuterium, a substituted or unsubstituted methyl group, a substituted or unsubstituted ethyl group, a substituted or unsubstituted phenyl group, a substituted or unsubstituted biphenyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, a substituted or unsubstituted fluorenyl group, a substituted or unsubstituted silyl group, or a cyano group.

In an implementation, the compound may be substituted with at least one deuterium atom. The number of substituted deuterium atoms may be 1 to the maximum number of hydrogens in the chemical formula, e.g., from 1 to 40 or from 1 to 30.

In an implementation, the compound may be represented by Chemical Formula 1A or 1B.

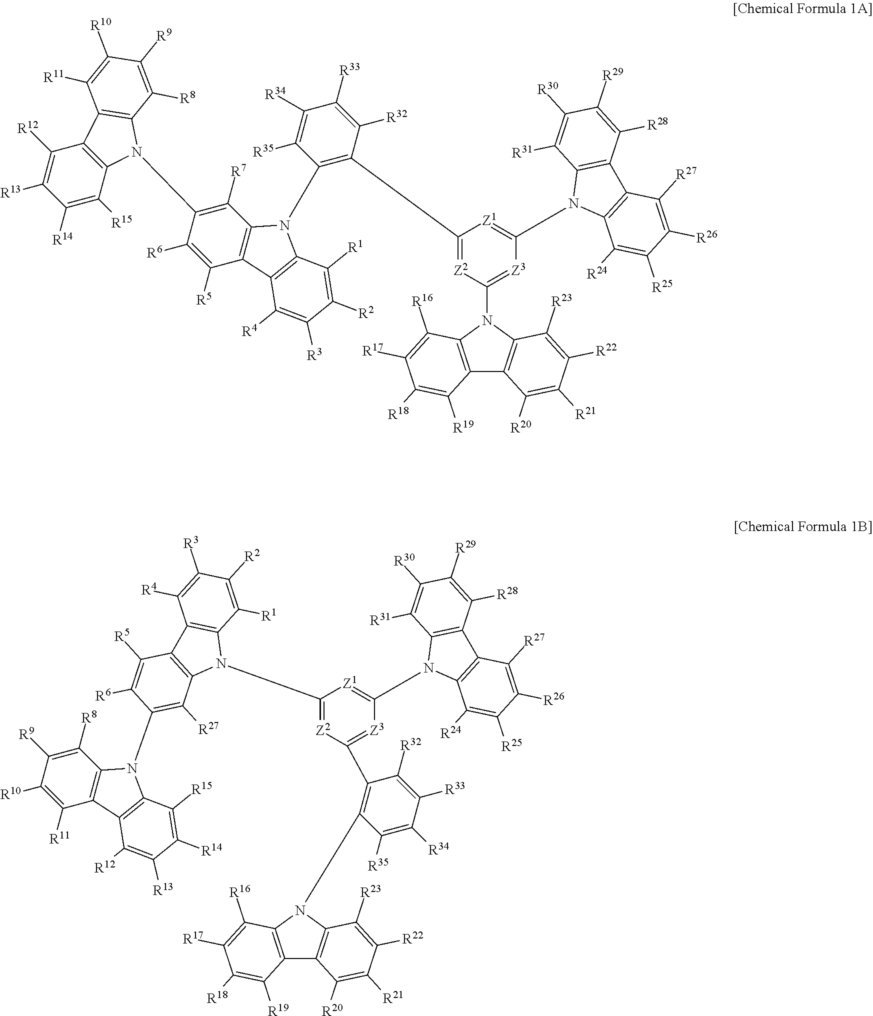

In Chemical Formula 1A or 1B, Z¹ to Z³, and R¹ to R³¹ may be defined the same as those described above.

R³² to R³⁵ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, excluding a carbazolyl group, a substituted or unsubstituted silyl group, a substituted or unsubstituted amine group, a halogen, a cyano group, or a combination thereof.

The compound may be, e.g., a compound of Group 1.

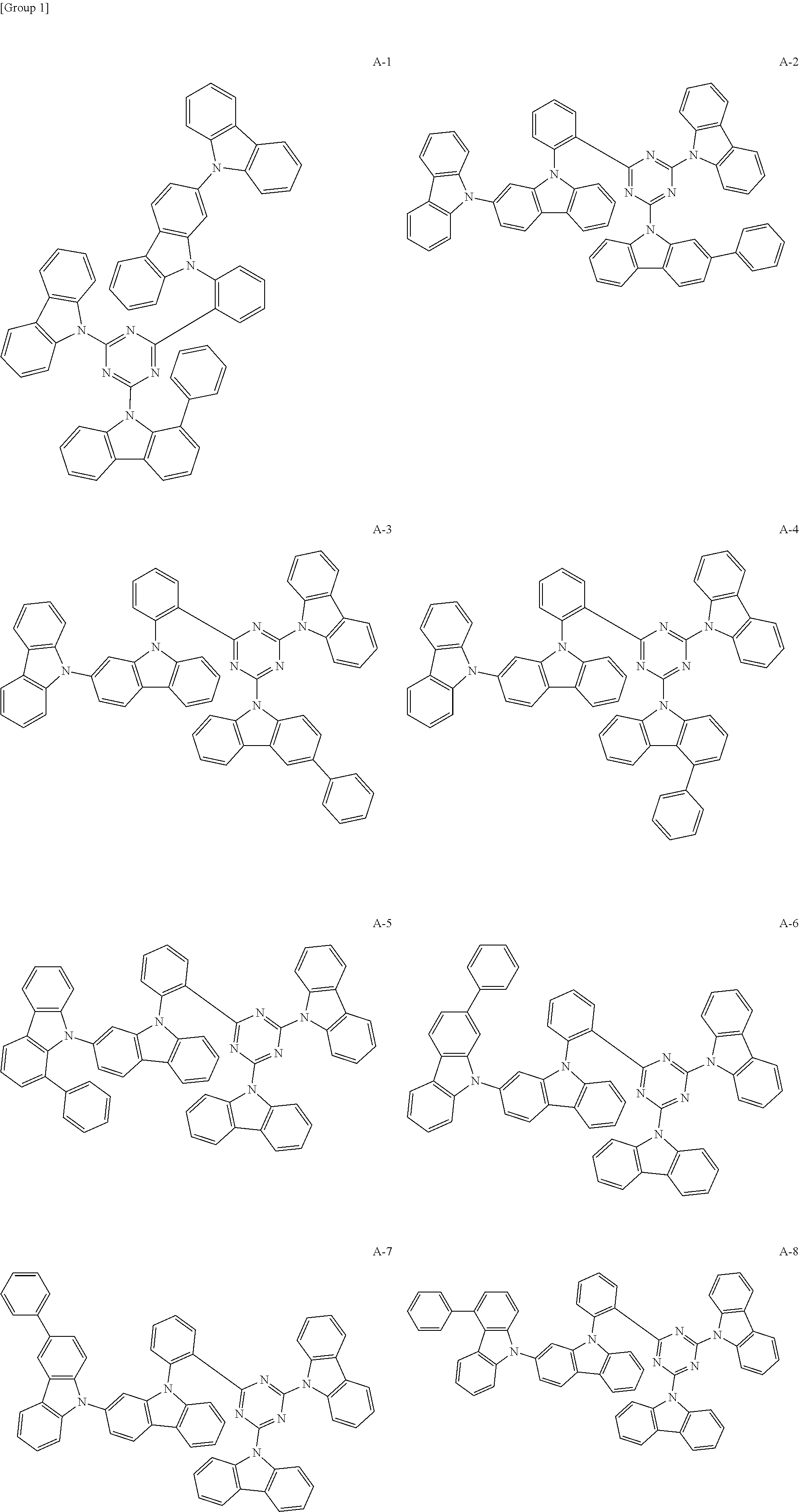

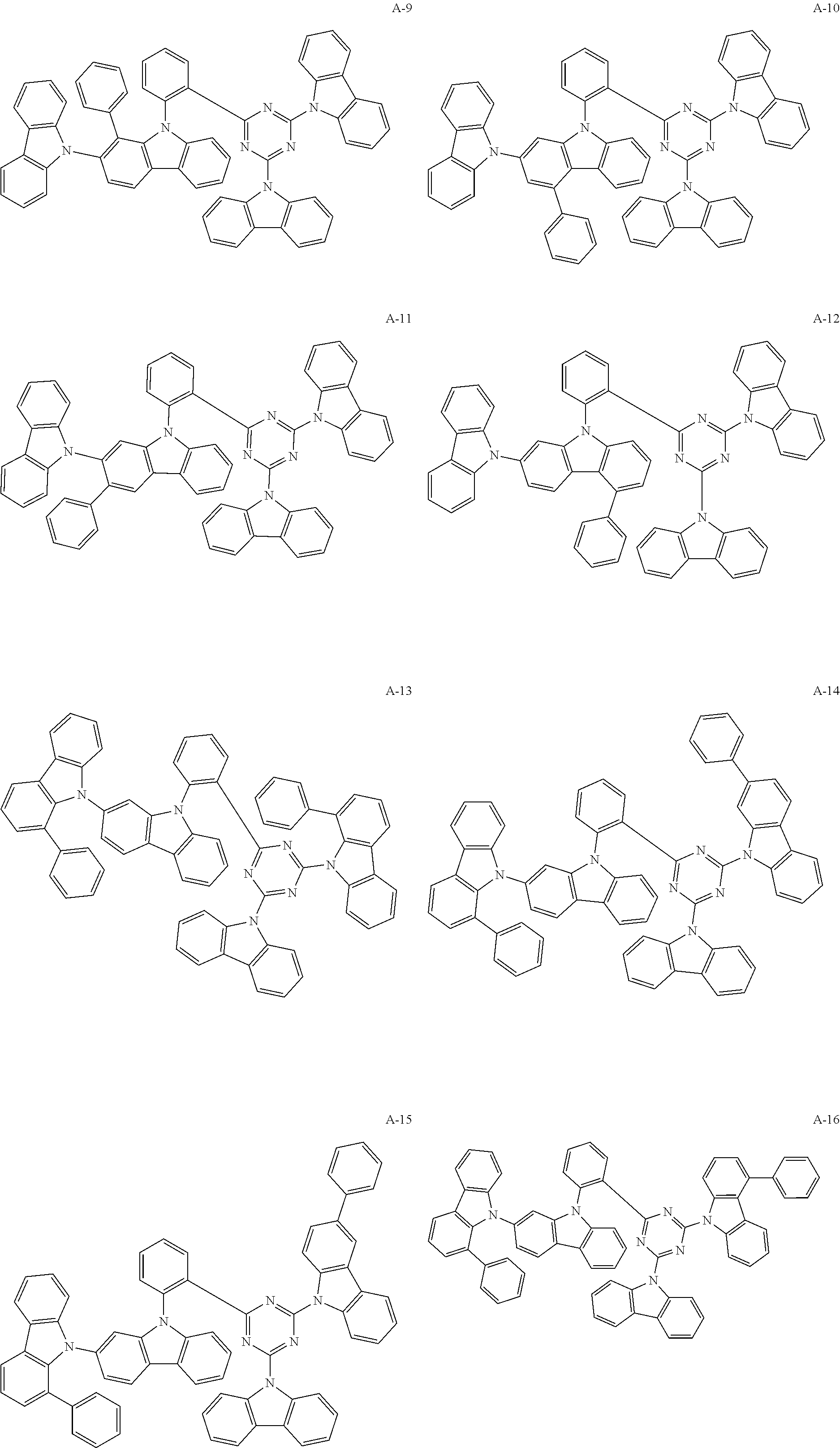

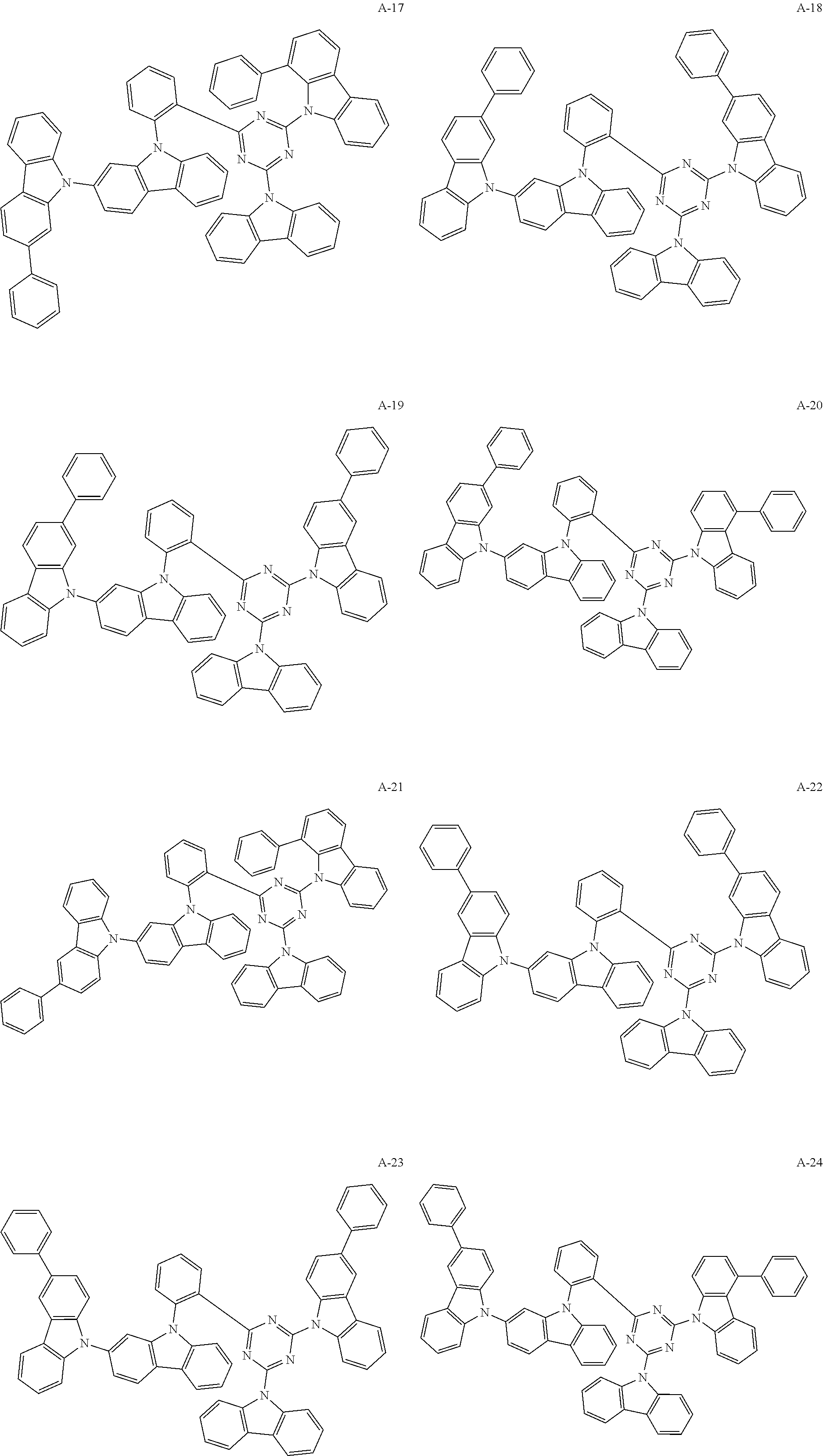

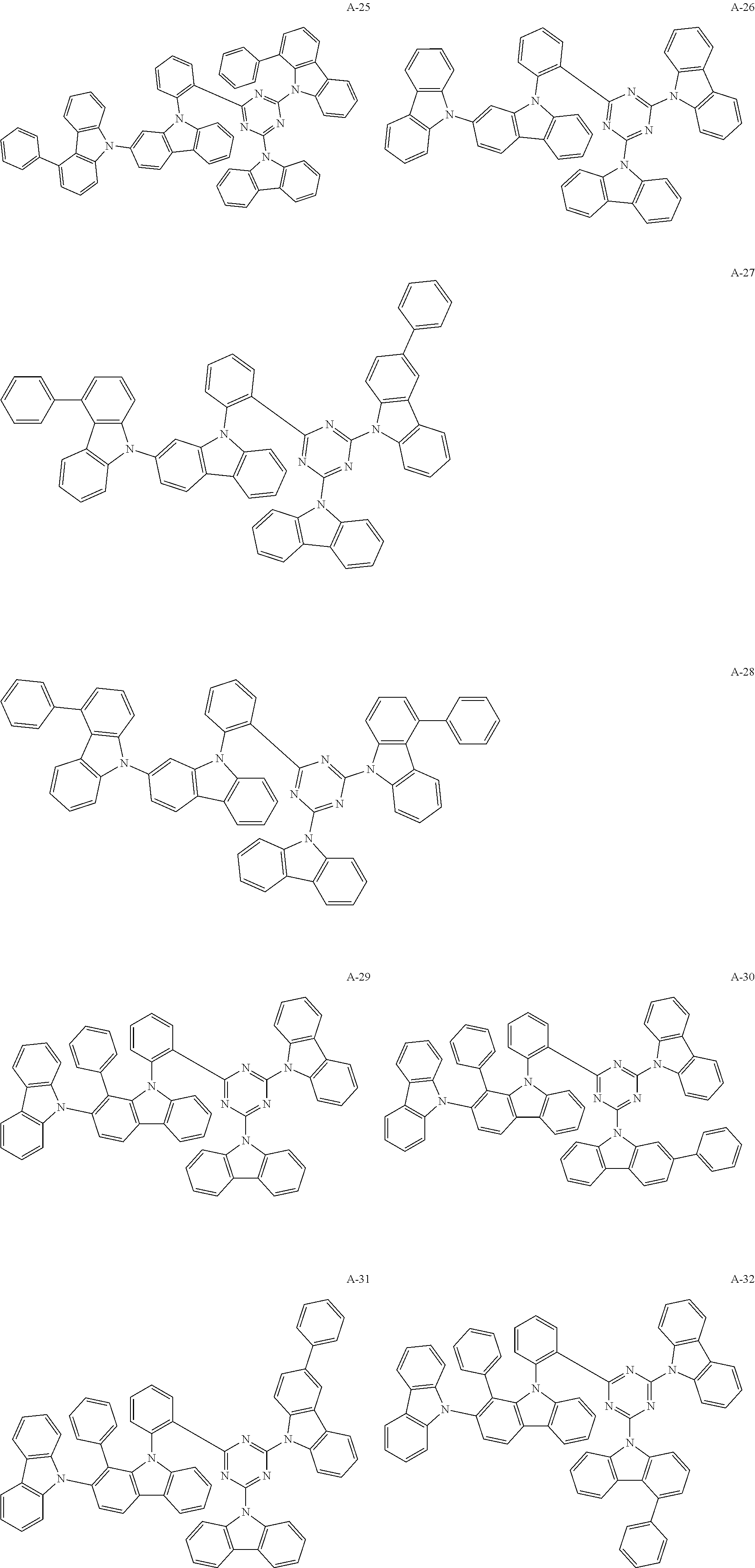

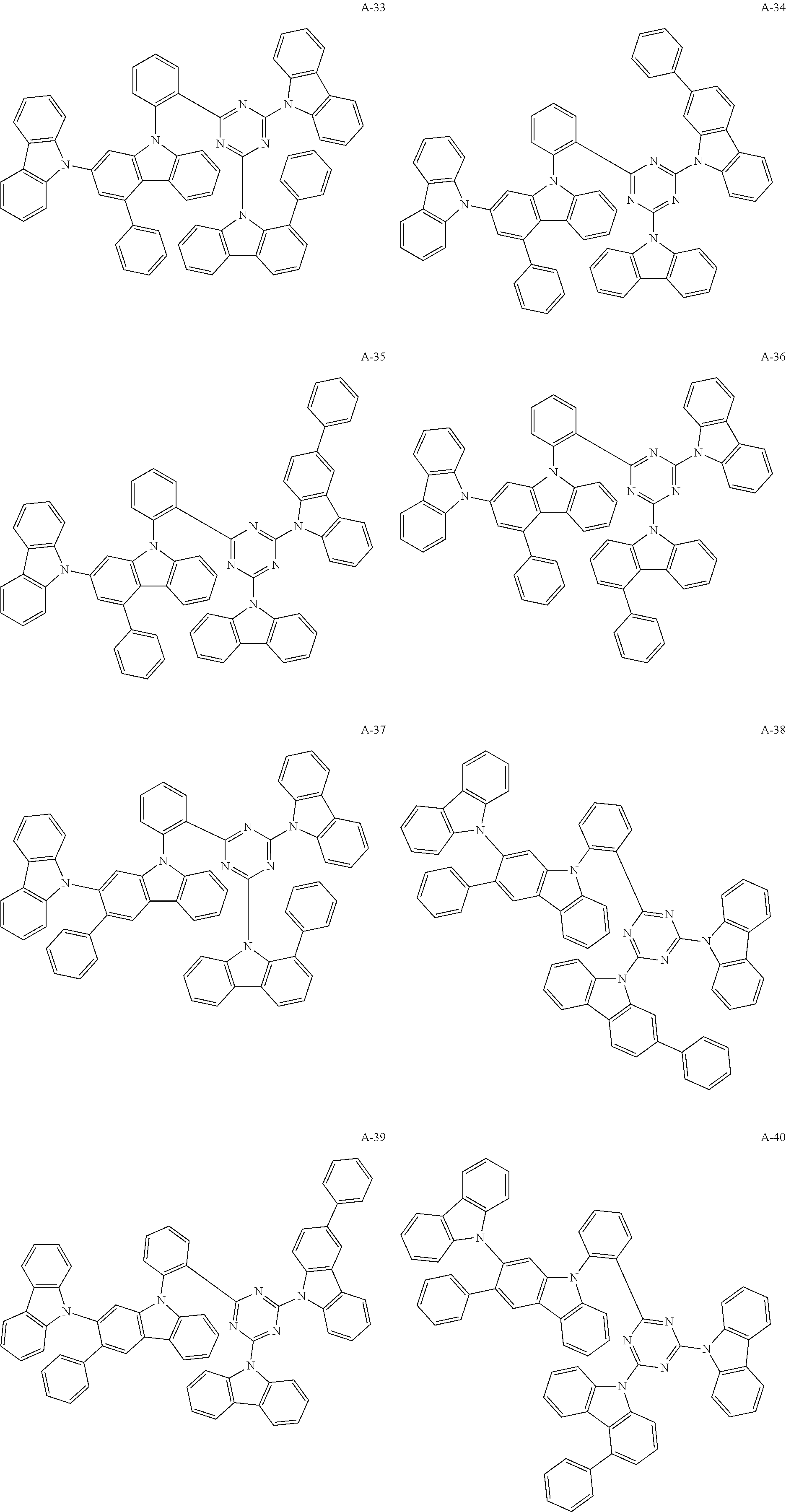

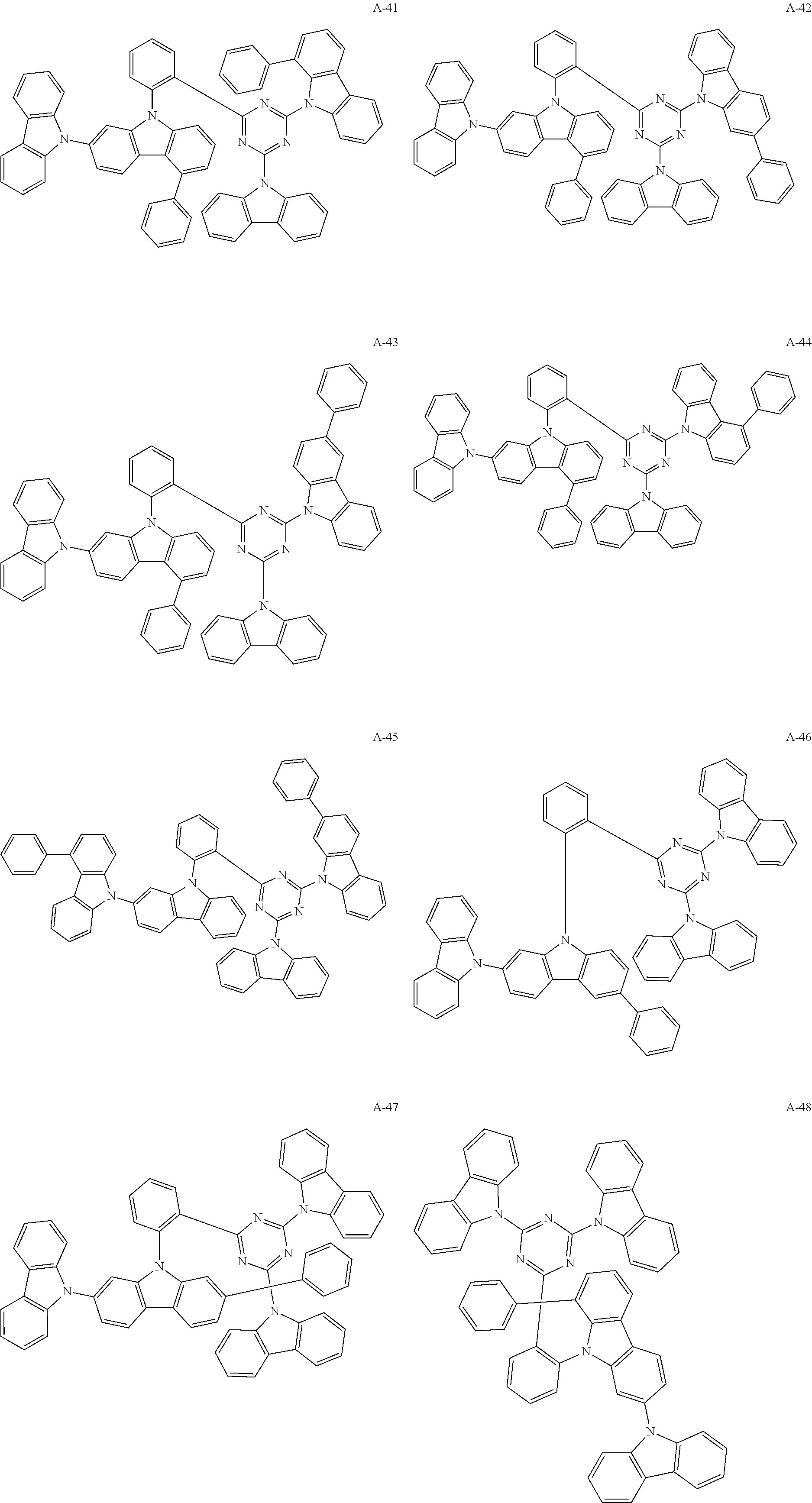

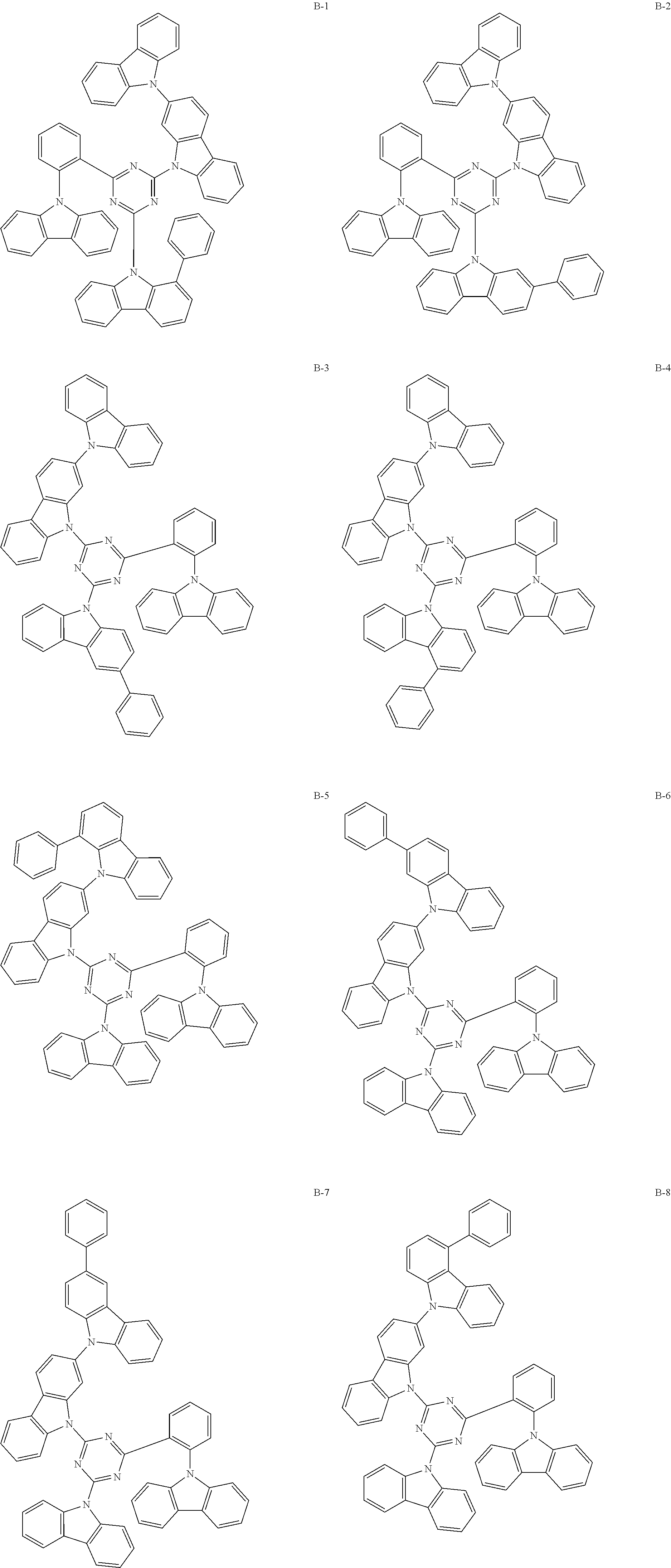

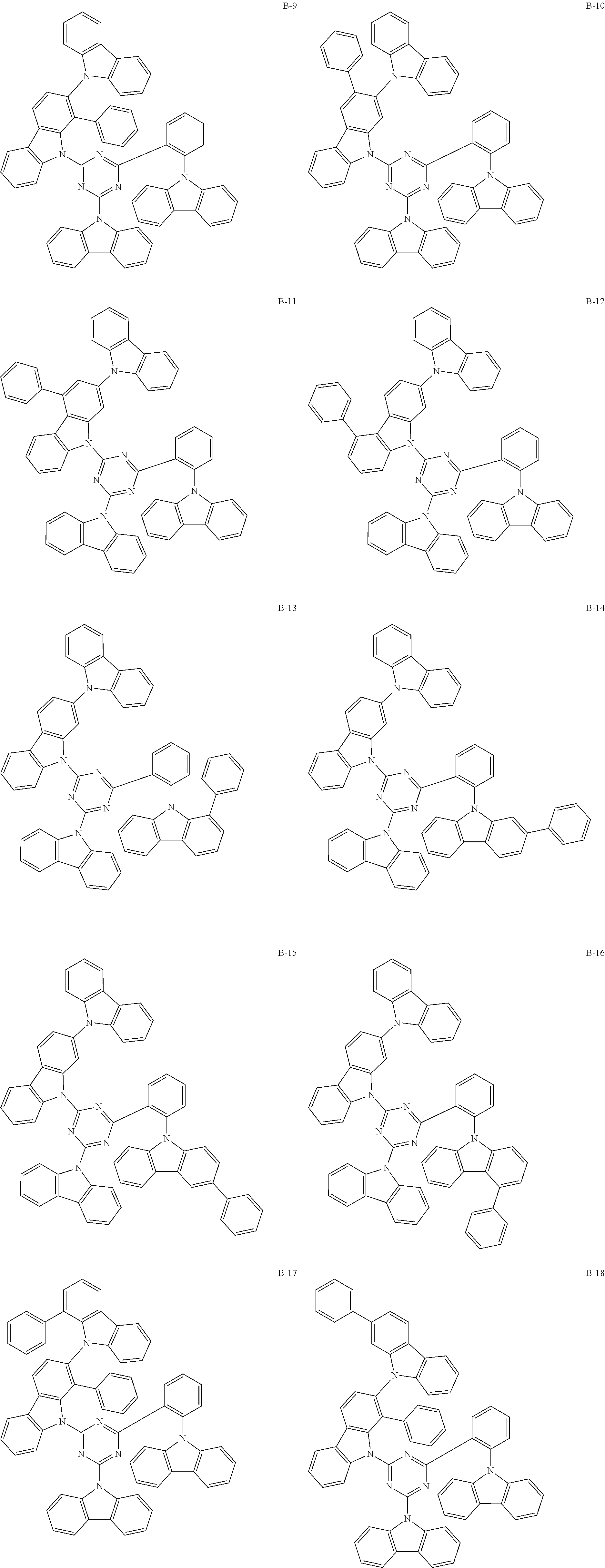

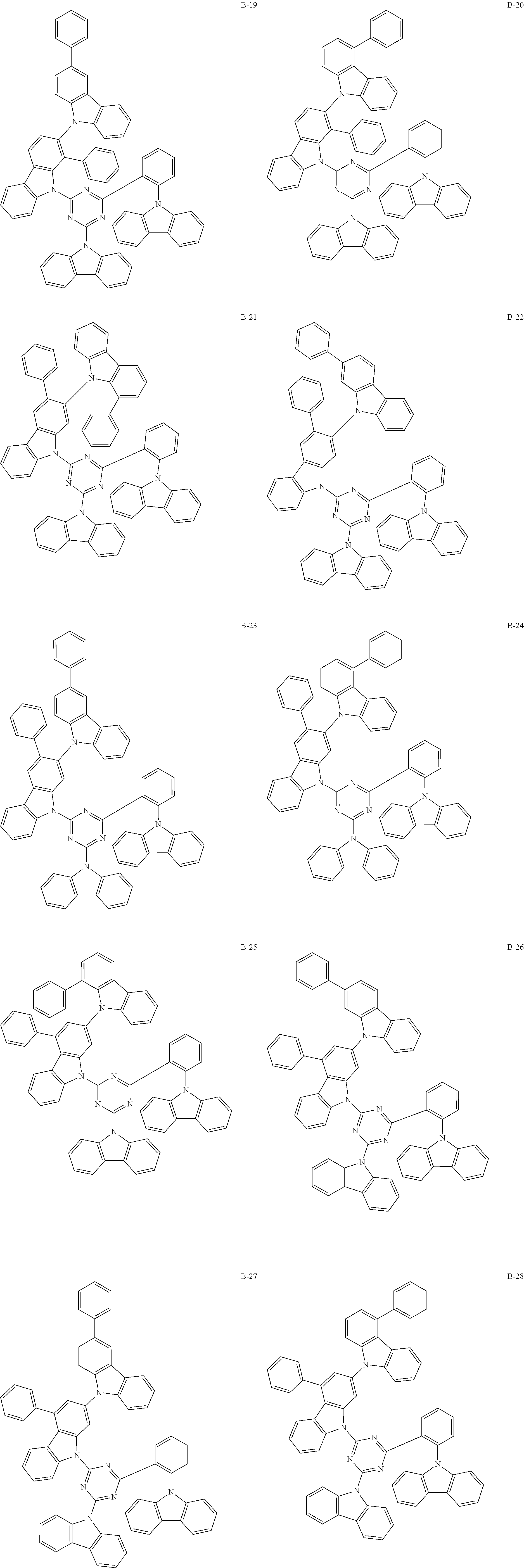

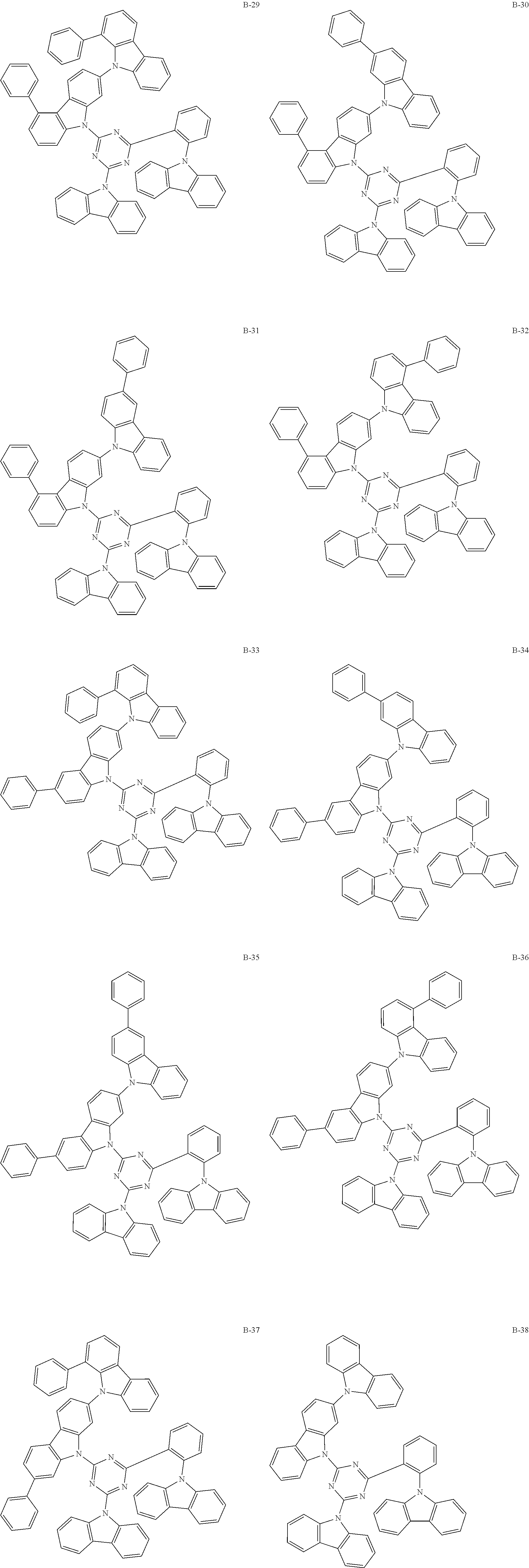

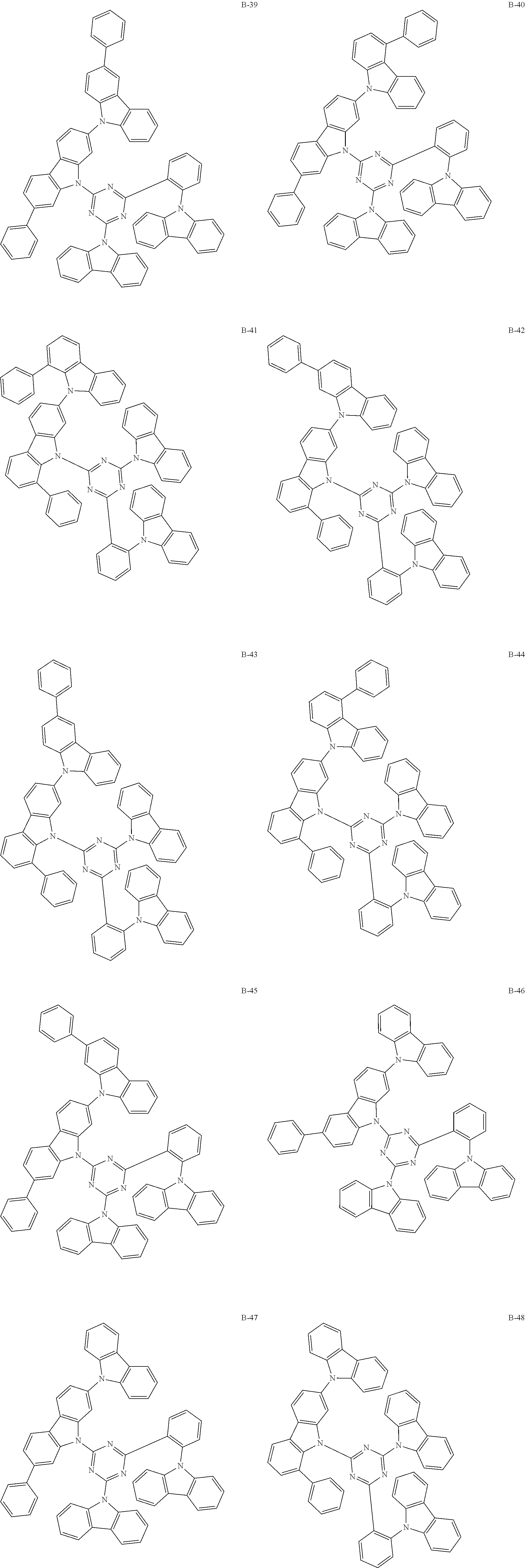

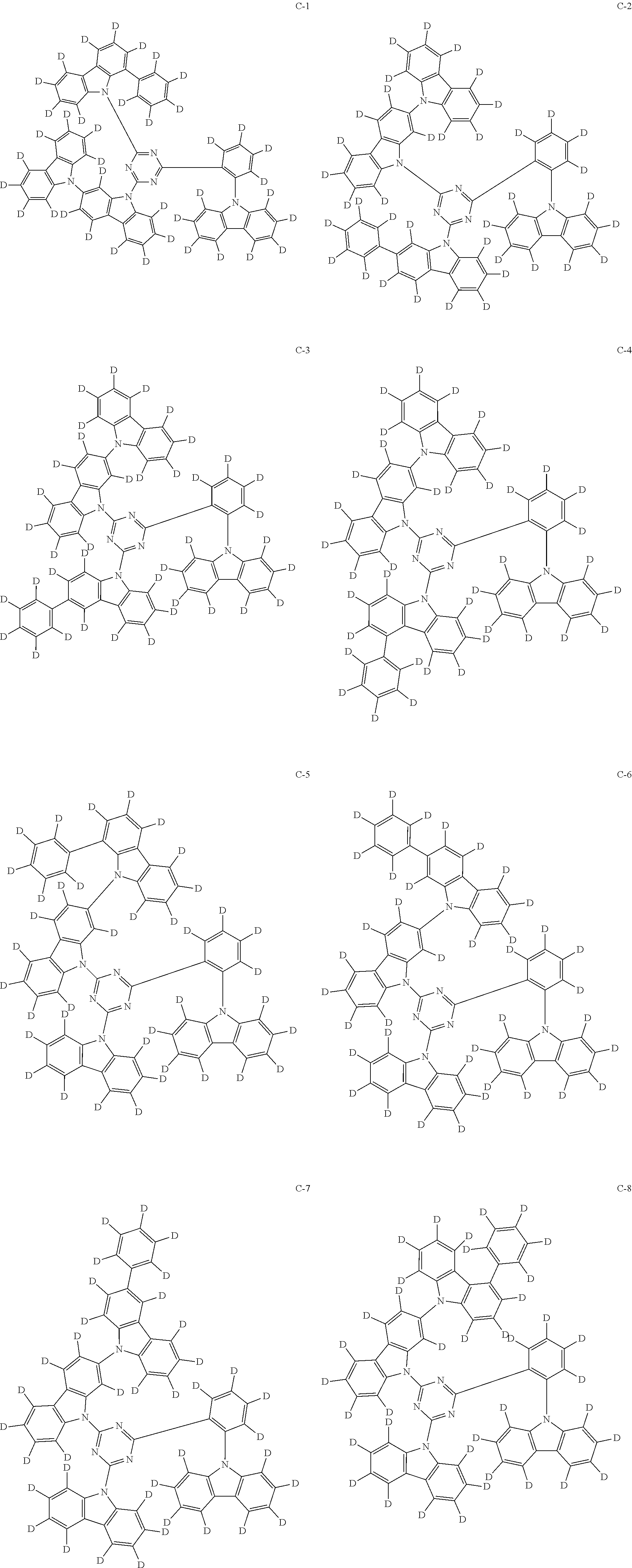

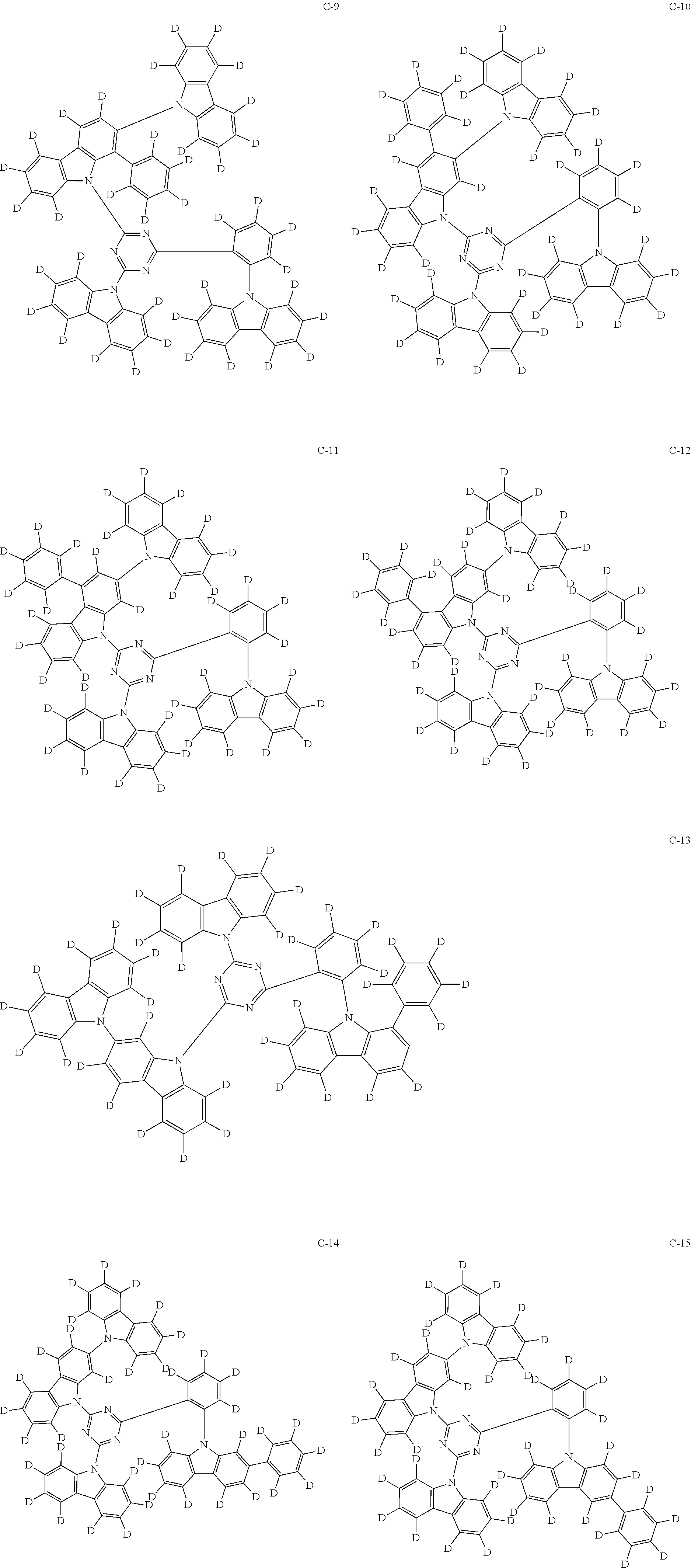

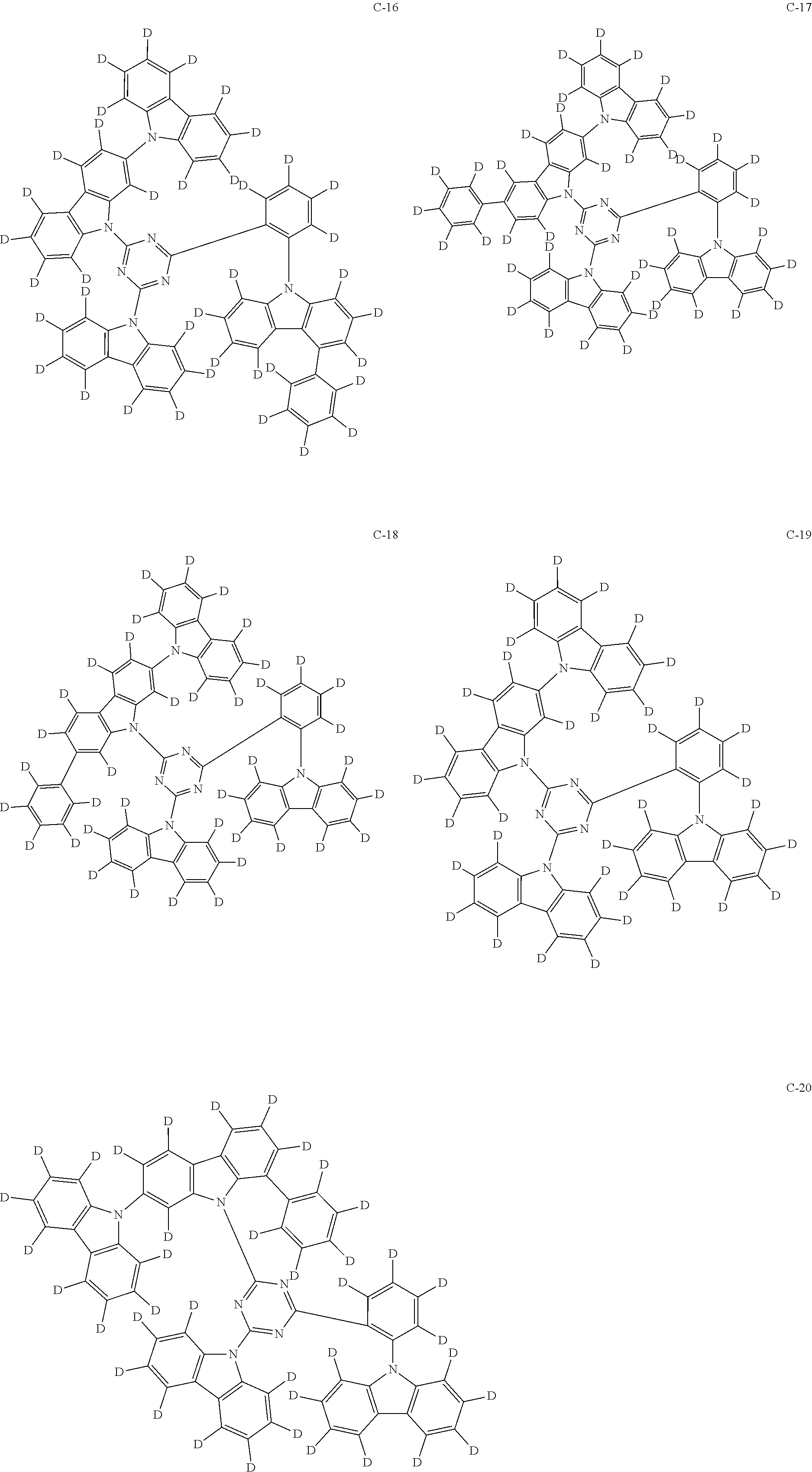

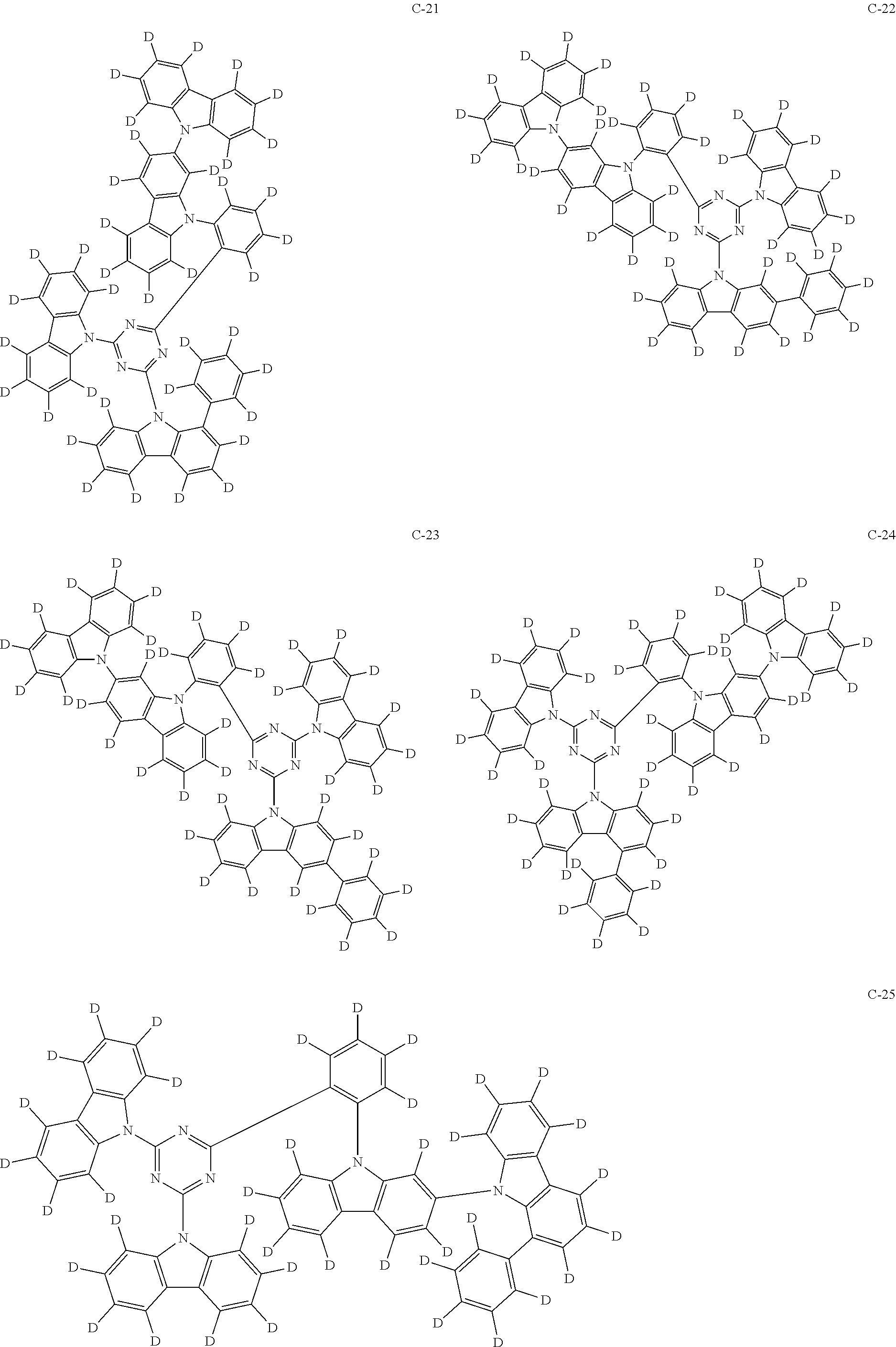

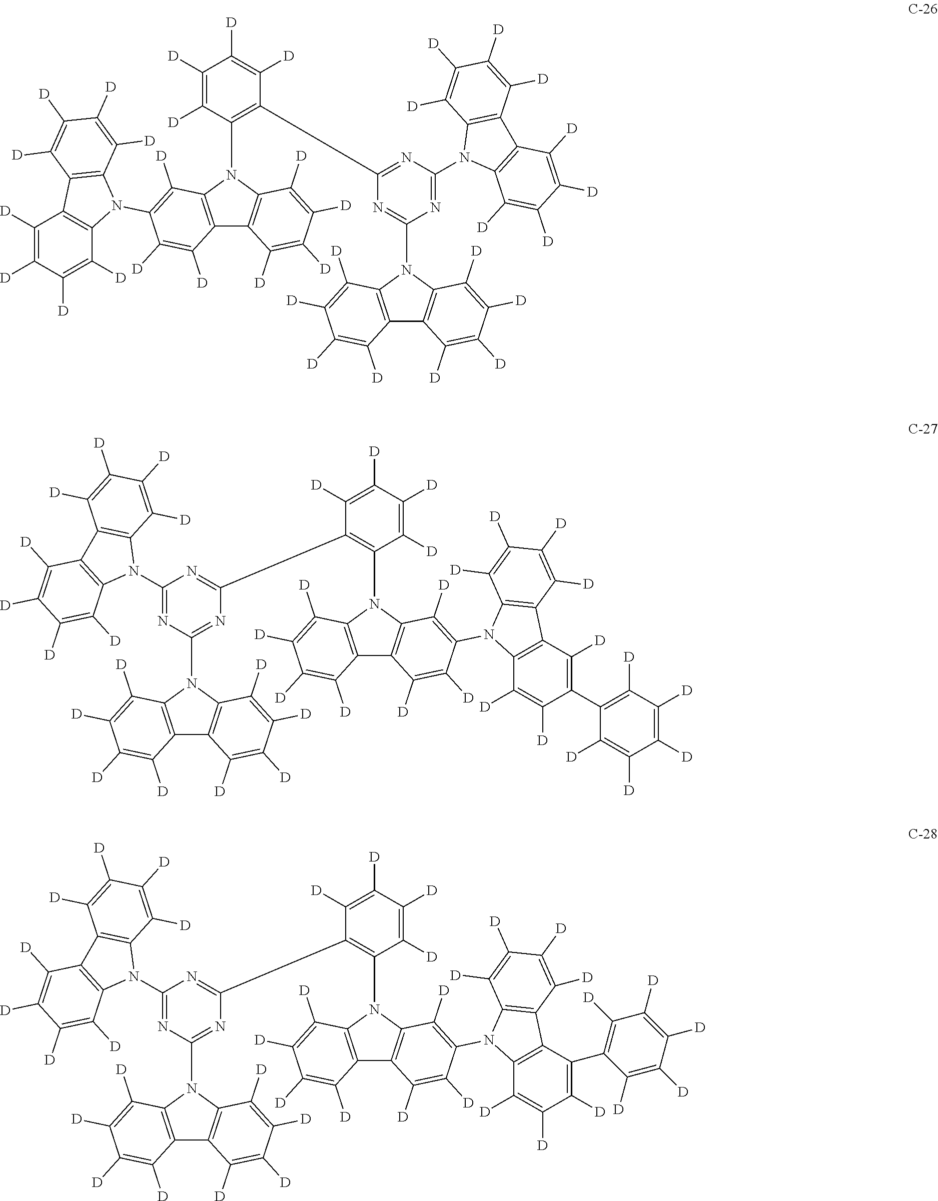

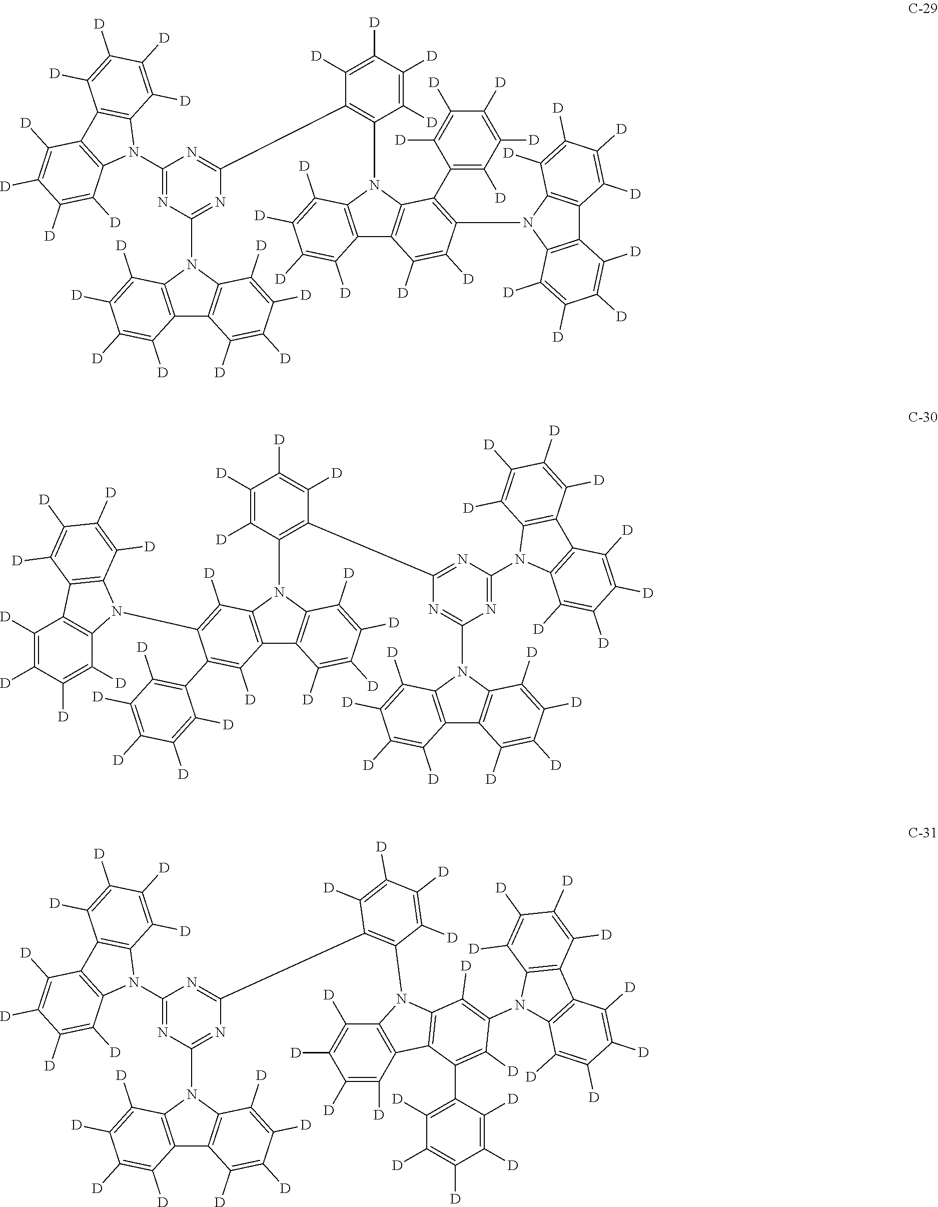

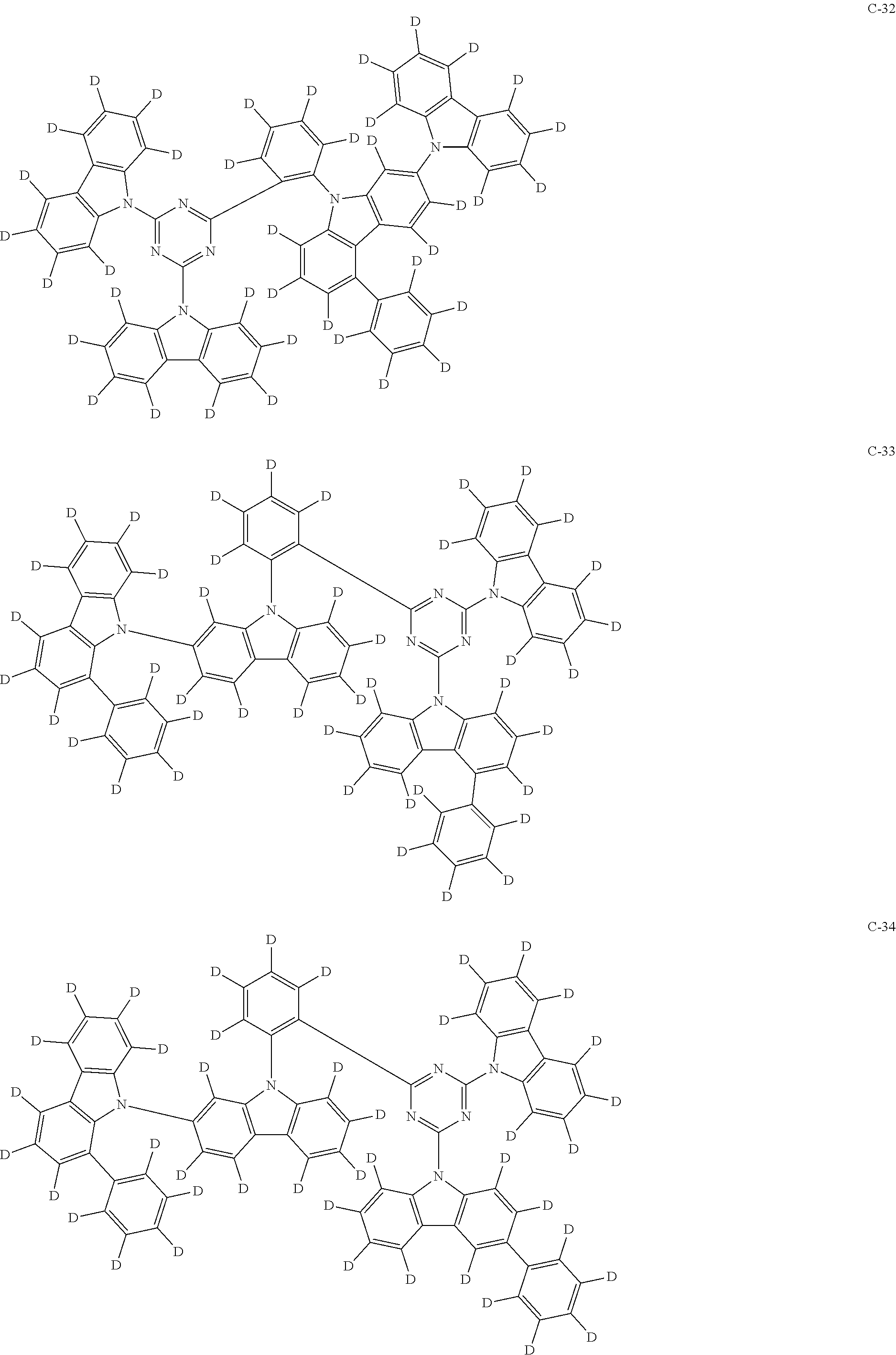

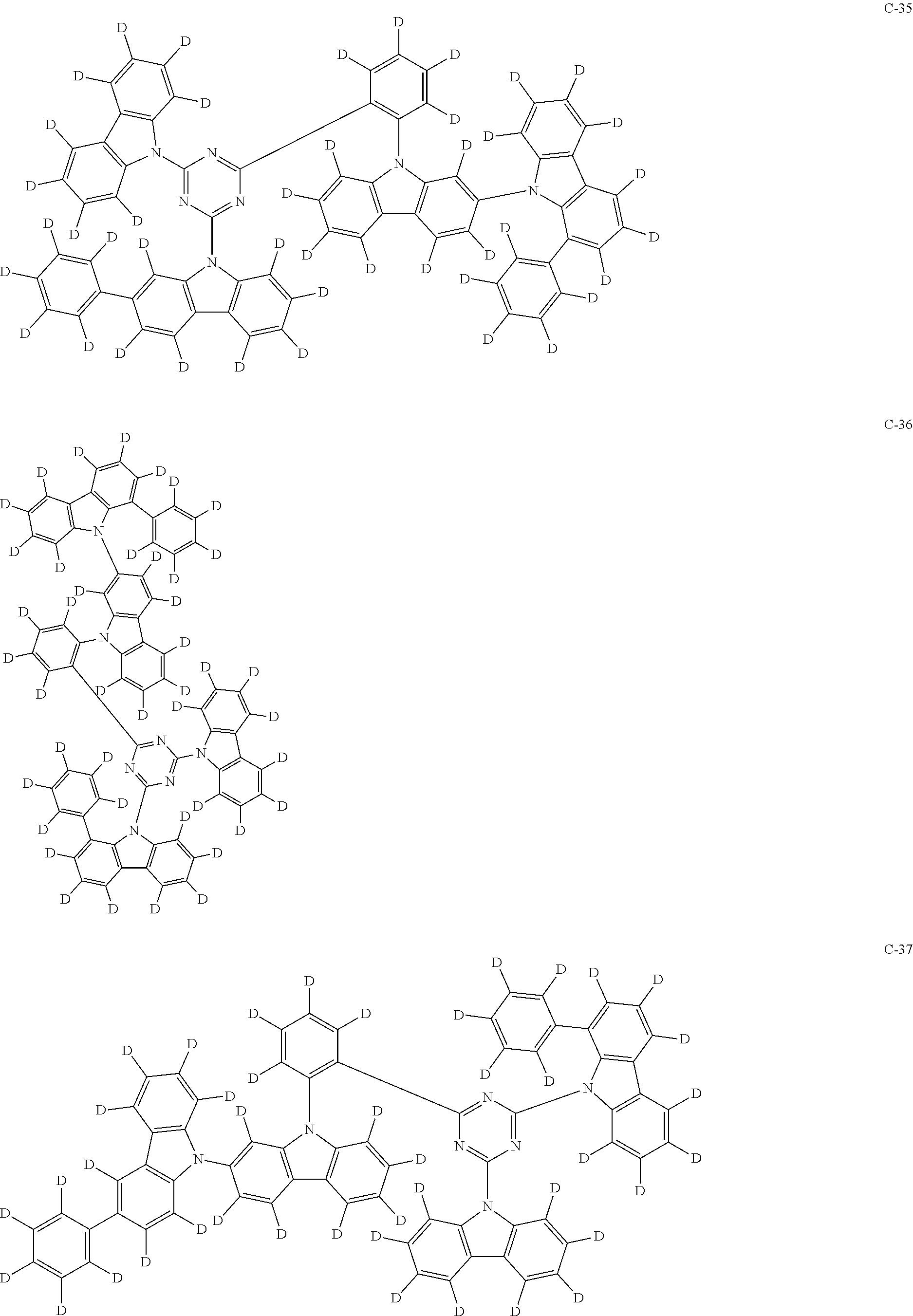

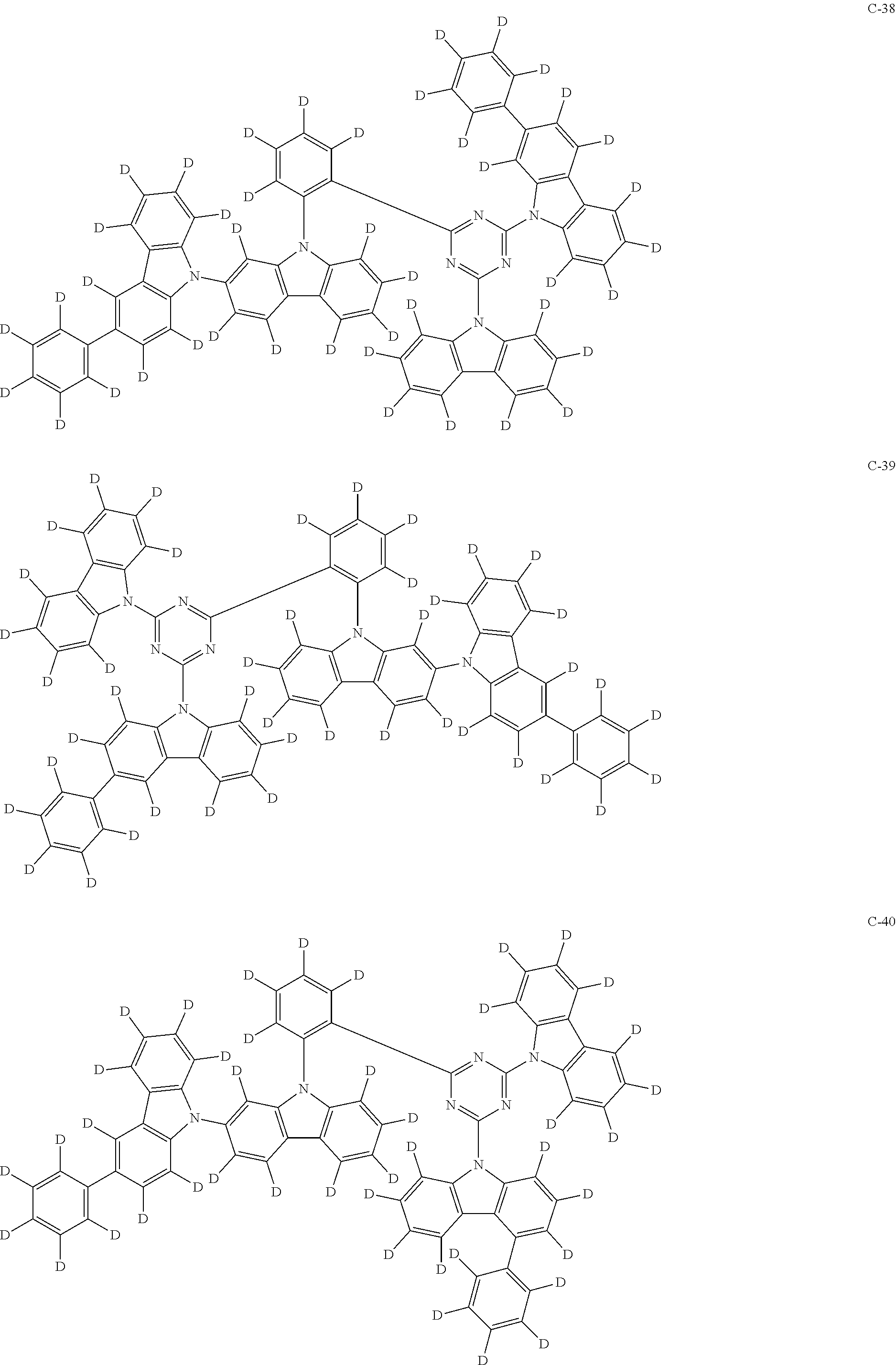

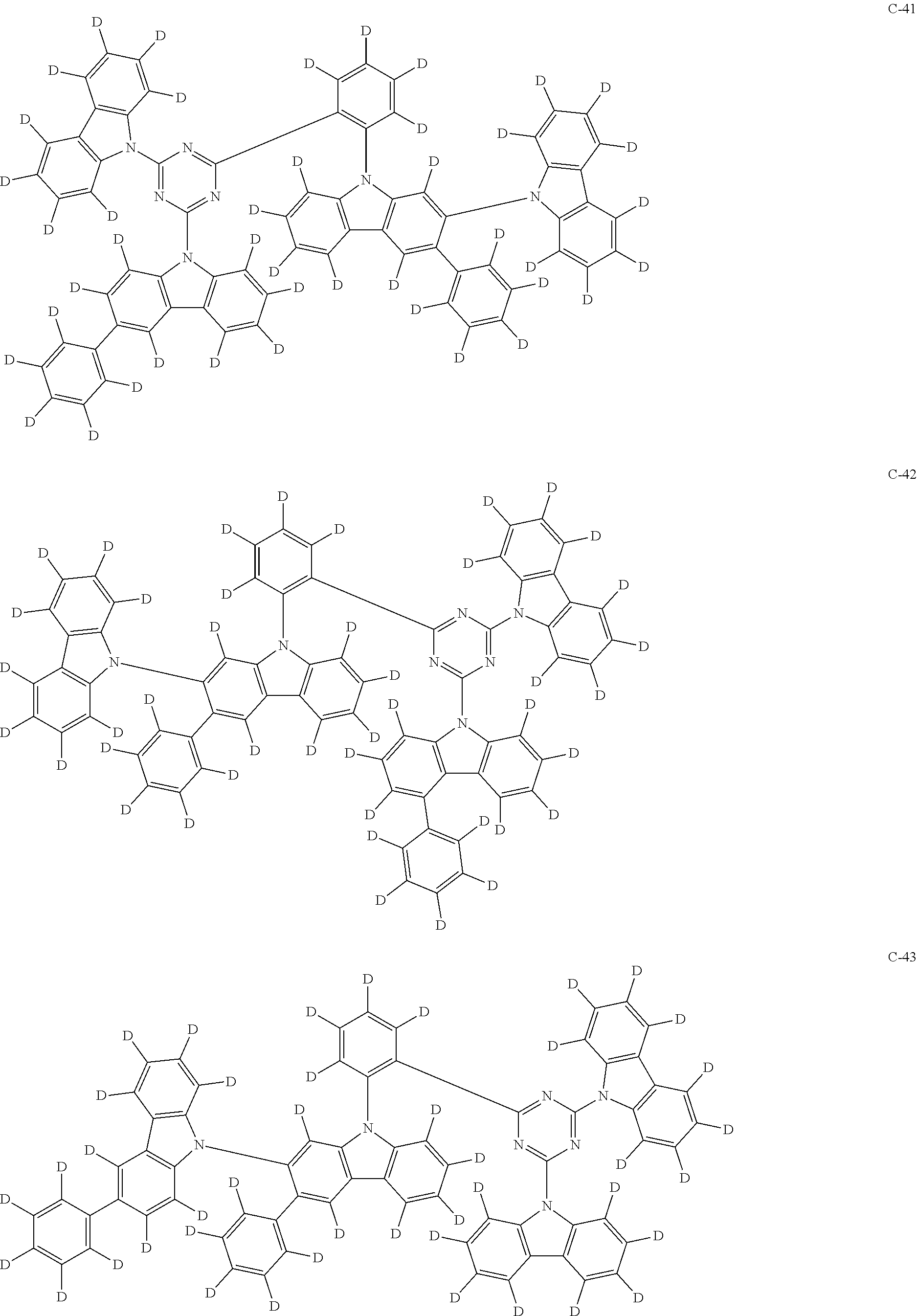

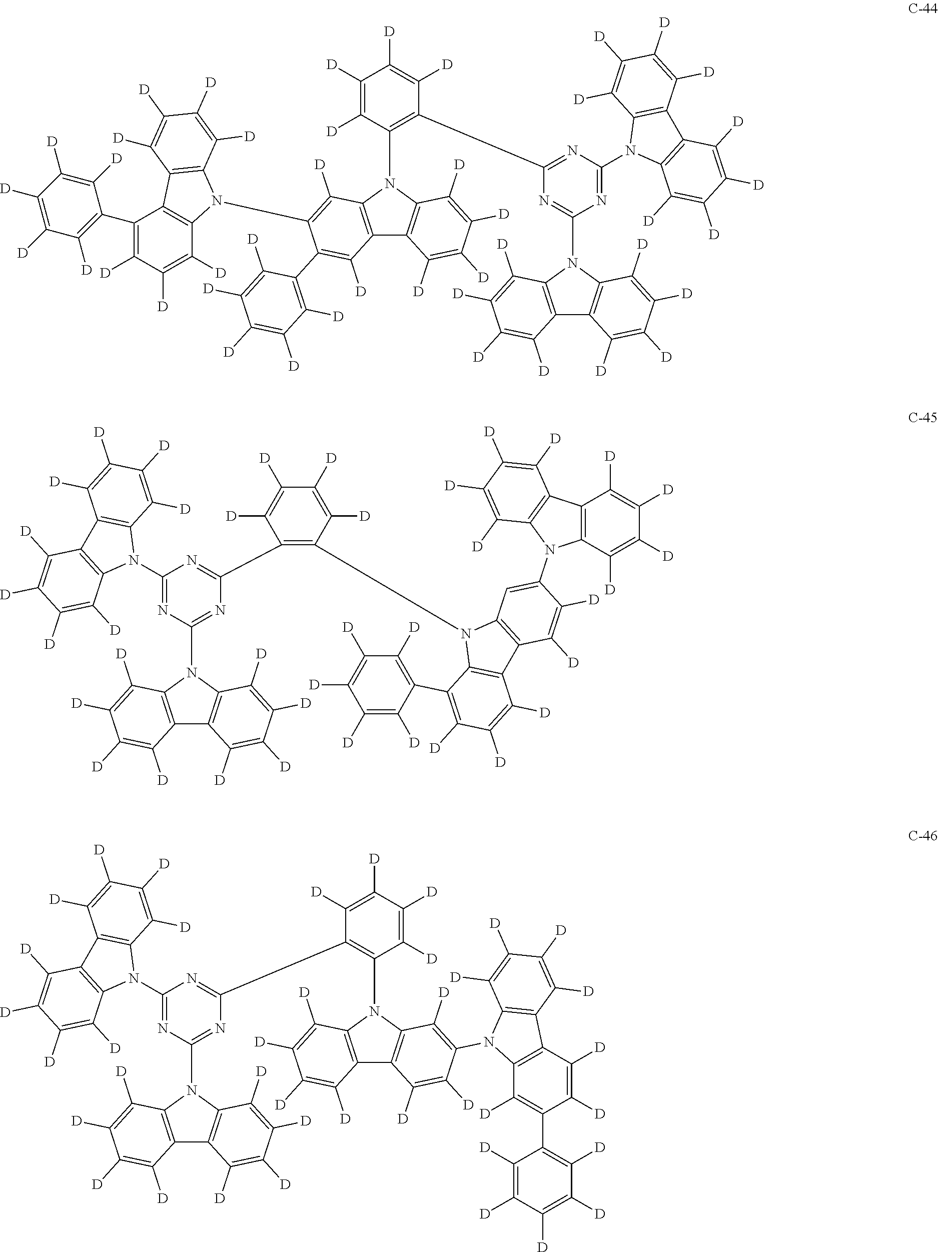

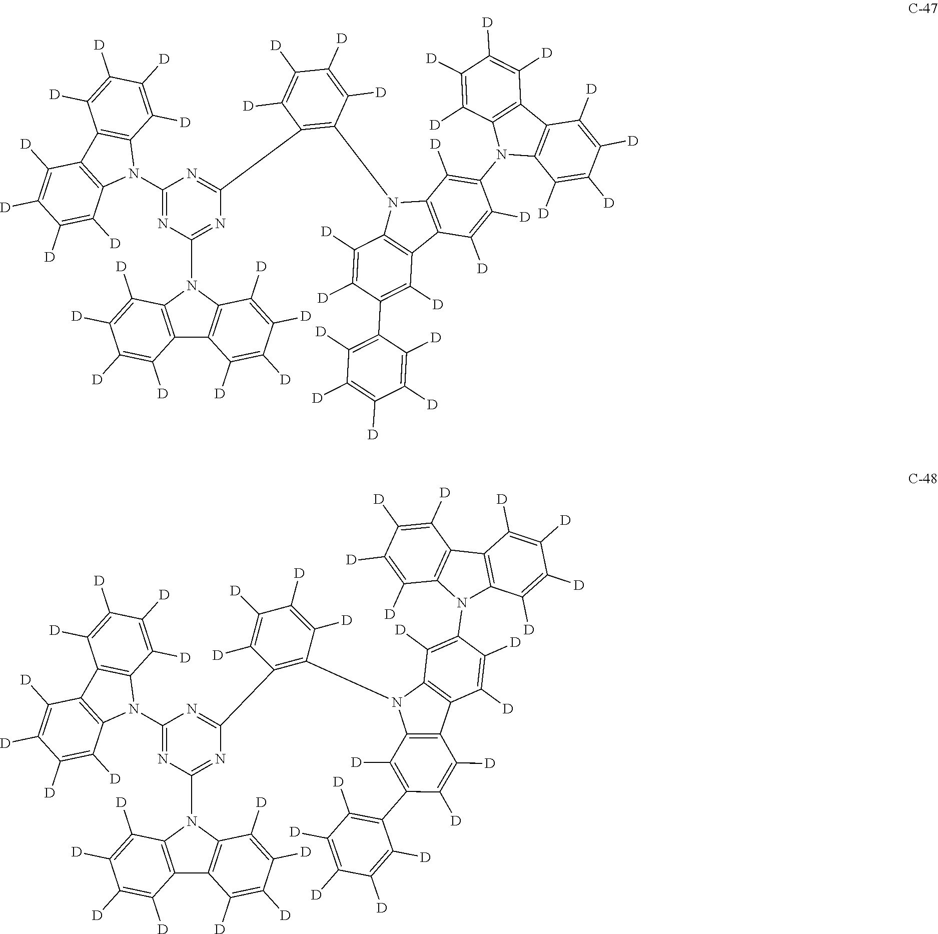

The aforementioned compound may be used as a composition together with other compounds to exhibit a synergistic effect in improving the performance of organic optoelectronic devices such as organic light emitting diodes.

A composition for an organic optoelectronic device according to some example embodiments may include, e.g., the aforementioned compound (a first compound) and a second compound that is different from the first compound.

The second compound may include, e.g., at least one carbazole moiety, and may have, e.g., a structure in which two or more carbazole moieties are directly or indirectly bonded, or may include an indolocarbazole moiety.

In an implementation, the second compound may be represented by Chemical Formula 2.

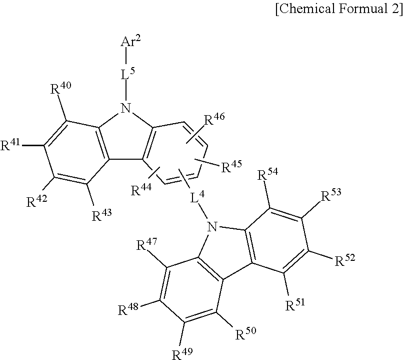

In Chemical Formula 2, L⁴ and L⁵ may each independently be or include, e.g., a single bond, a substituted or unsubstituted C6 to C30 arylene group, a substituted or unsubstituted divalent C3 to C30 heterocyclic group, or a combination thereof.

Ar² may be or include, e.g., a substituted or unsubstituted C6 to C30 aryl group or a substituted or unsubstituted C2 to C30 heterocyclic group.

R⁴⁰ to R⁵⁴ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

R⁴⁰ to R⁵⁴ may each be separately present or two adjacent ones may be linked to each other to form a ring.

In an implementation, the second compound represented by Chemical Formula 2 may be represented by one of Chemical Formulas 2A to 2D.

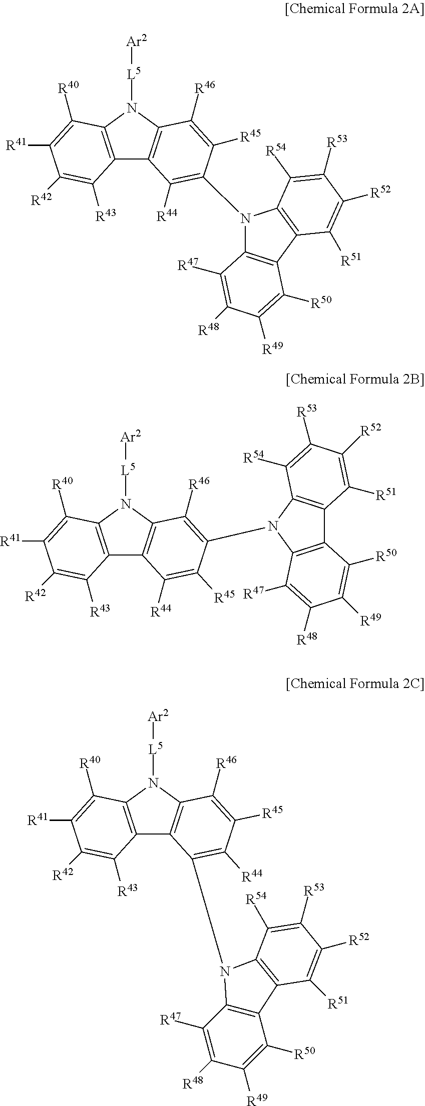

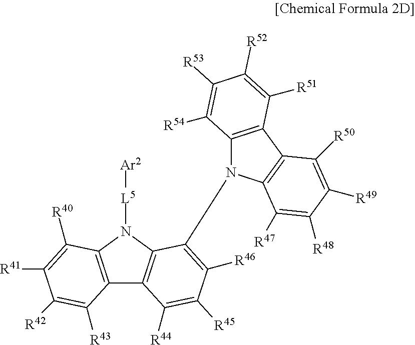

In Chemical Formulas 2A to 2D, L⁵, Ar², and R⁴⁰ to R⁵⁴ may be defined the same as those described above.

In an implementation, in Chemical Formulas 2 and 2A to 2D, L⁵ may be, e.g., a single bond, a substituted or unsubstituted phenylene group, a substituted or unsubstituted biphenylene group, or a substituted or unsubstituted terphenylene group.

In an implementation, in Chemical Formulas 2 and 2A to 2D, Ar² may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

In an implementation, in Chemical Formulas 2 and 2A to 2D, R⁴⁰ to R⁵⁴ may each independently be, e.g., hydrogen, deuterium, a substituted or unsubstituted methyl group, a substituted or unsubstituted ethyl group, a substituted or unsubstituted phenyl group, a substituted or unsubstituted biphenyl group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, a substituted or unsubstituted fluorenyl group, a substituted or unsubstituted silyl group, or a cyano group.

In an implementation, in Chemical Formulas 2 and 2A to 2D, at least one of Ar² and R⁴⁰ to R⁵⁴ may be, e.g., a substituted or unsubstituted carbazolyl group.

In an implementation, at least one of the substituents of Chemical Formulas 2 and 2A to 2D may be substituted with deuterium. The number of substituted deuterium atoms may be 1 to the maximum number of hydrogens in the chemical formula, e.g., 1 to 40 or 1 to 30.

In an implementation, the second compound represented by Chemical Formula 2 may include, e.g., a compound of Group 2.

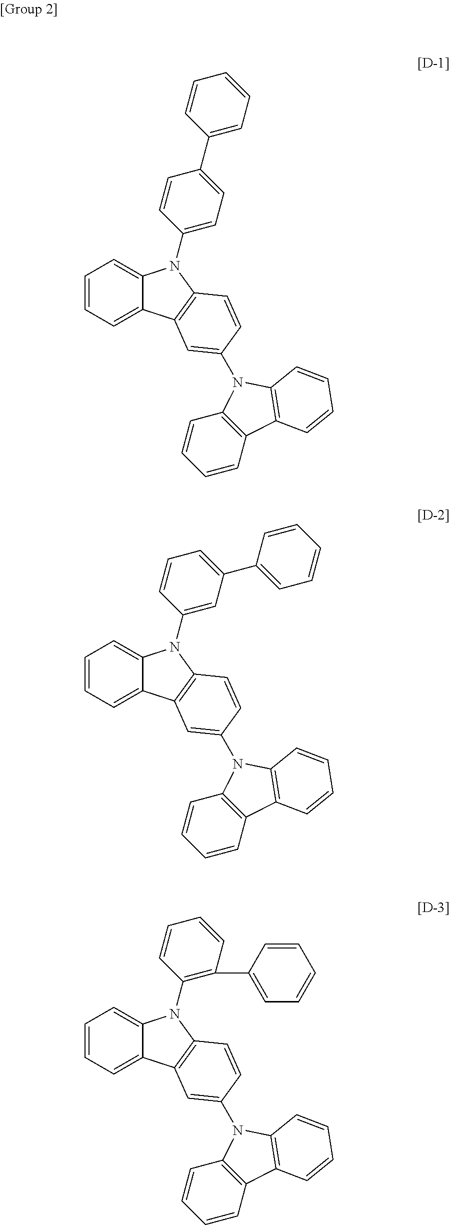 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1]

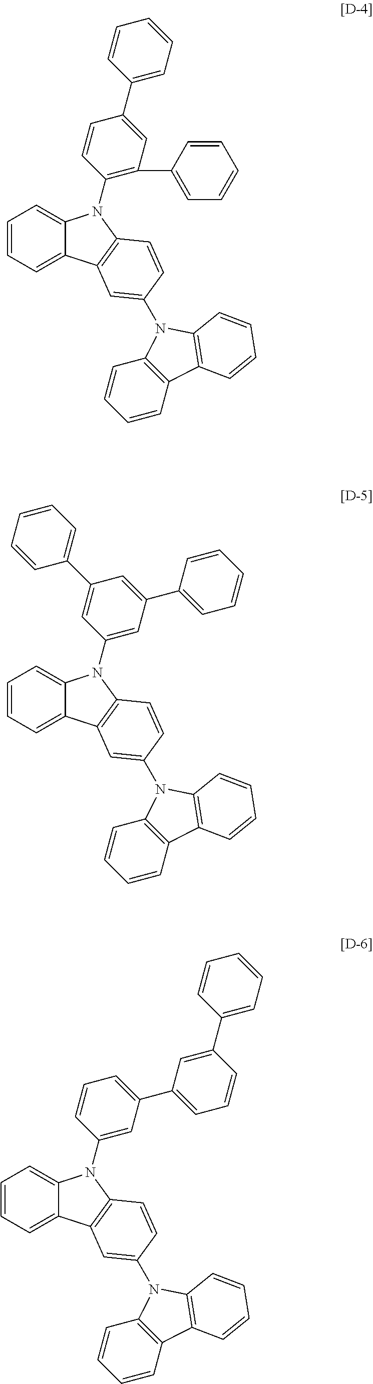 [SMILES: c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1 ; c1ccc(-c2cccc(-c3cccc(-n4c5ccccc5c5cc(-n6c7ccccc7c7ccccc76)ccc54)c3)c2)cc1]

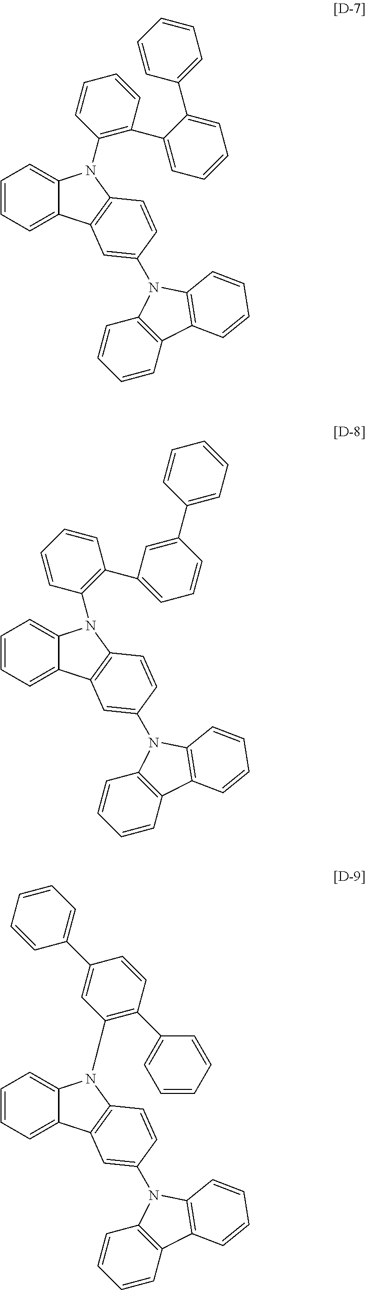 [SMILES: c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1]

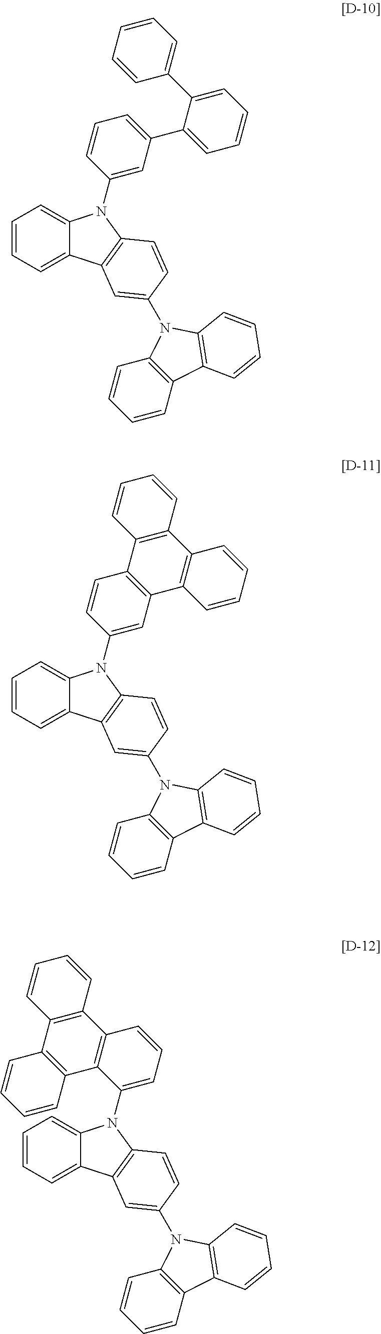 [SMILES: c1ccc(-c2ccccc2-c2cccc(-n3c4ccccc4c4cc(-n5c6ccccc6c6ccccc65)ccc43)c2)cc1 ; c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21]

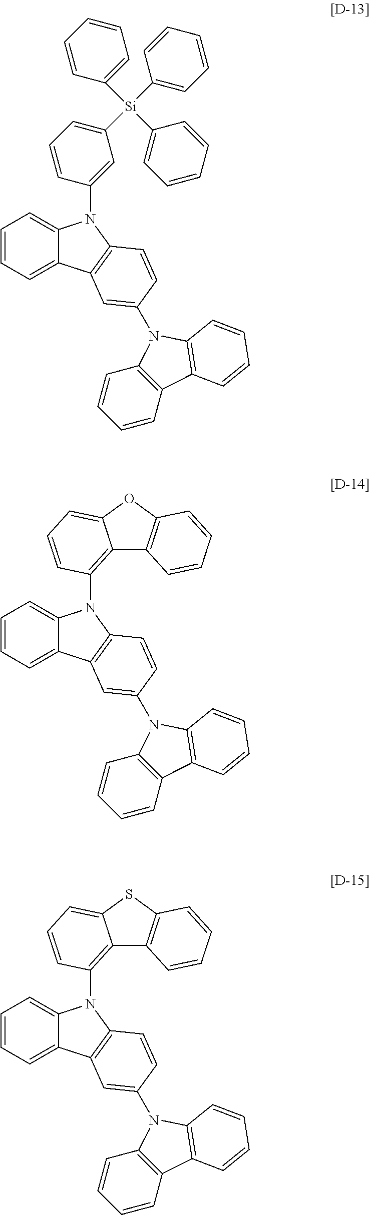 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

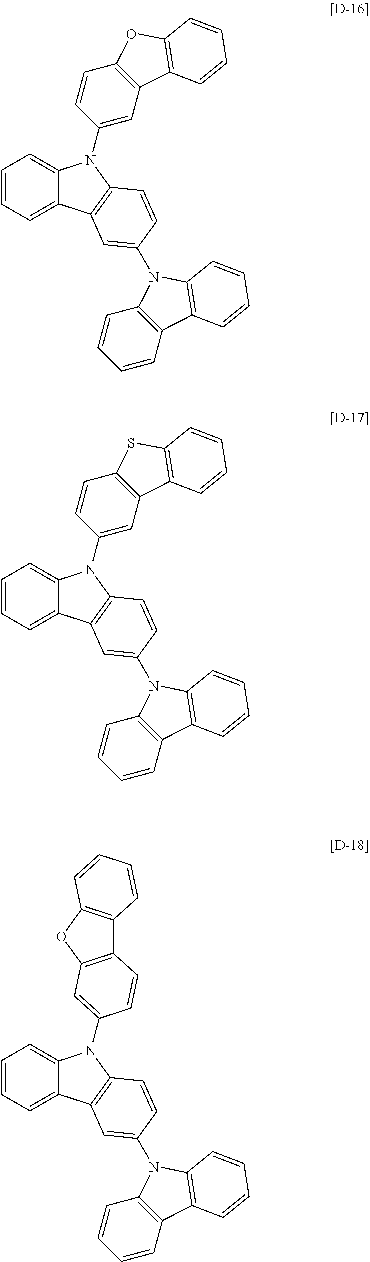 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12]

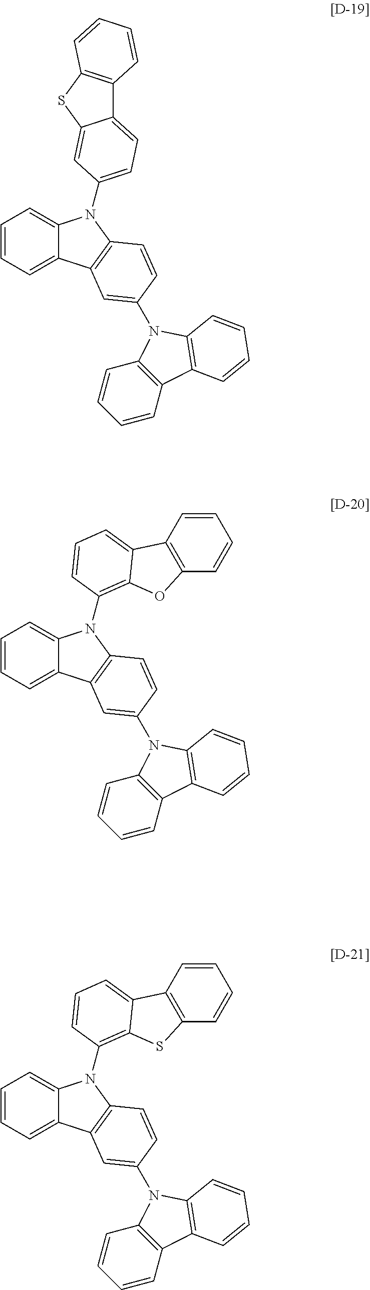 [SMILES: c1ccc2c(c1)sc1cc(-n3c4ccccc4c4cc(-n5c6ccccc6c6ccccc65)ccc43)ccc12 ; c1ccc2c(c1)oc1c(-n3c4ccccc4c4cc(-n5c6ccccc6c6ccccc65)ccc43)cccc12 ; c1ccc2c(c1)sc1c(-n3c4ccccc4c4cc(-n5c6ccccc6c6ccccc65)ccc43)cccc12]

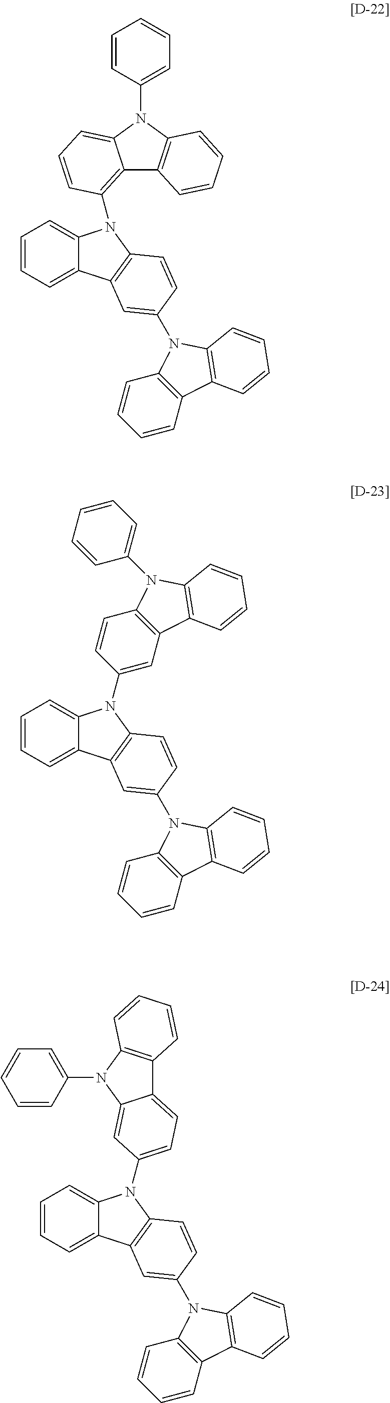 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1]

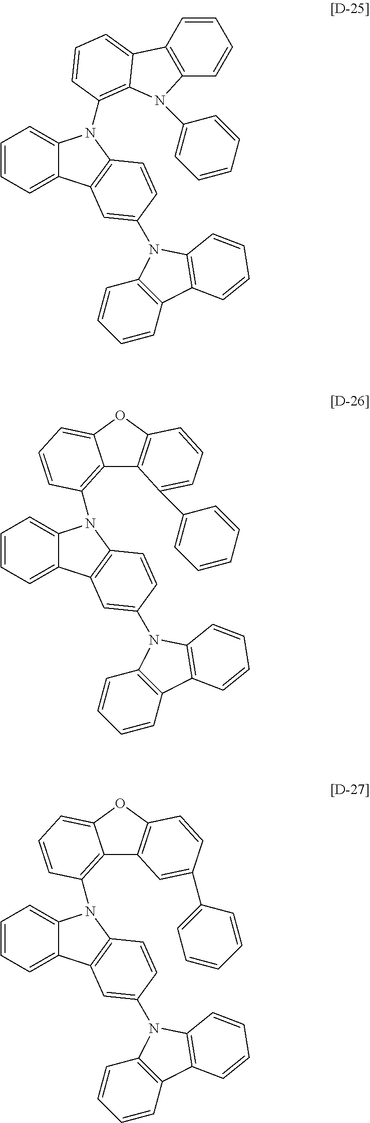 [SMILES: c1ccc(-n2c3ccccc3c3cccc(-n4c5ccccc5c5cc(-n6c7ccccc7c7ccccc76)ccc54)c32)cc1 ; c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1]

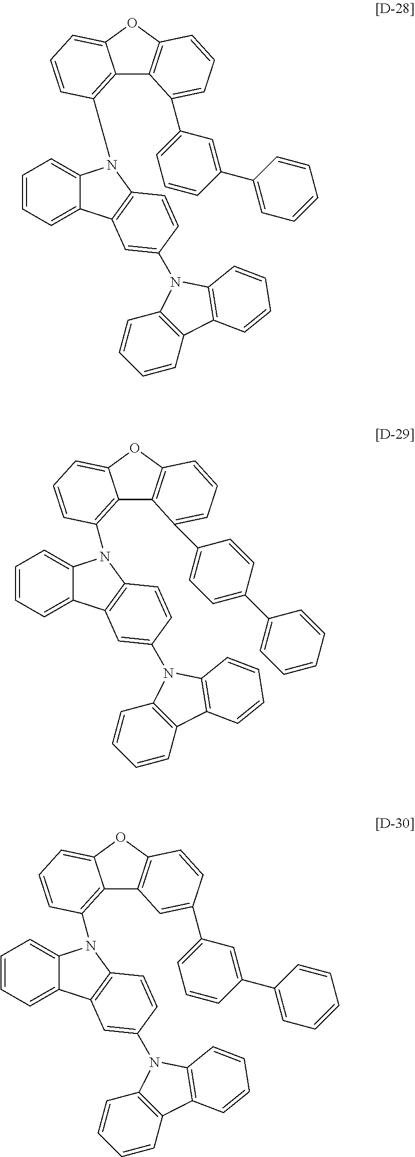 [SMILES: c1ccc(-c2cccc(-c3cccc4oc5cccc(-n6c7ccccc7c7cc(-n8c9ccccc9c9ccccc98)ccc76)c5c34)c2)cc1 ; c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1]

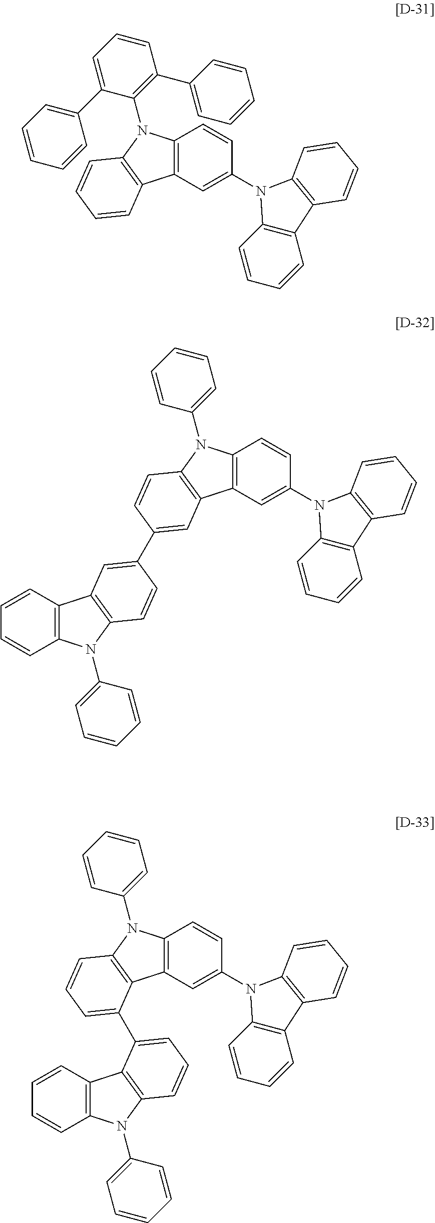 [SMILES: c1ccc(-c2cccc(-c3ccccc3)c2-n2c3ccccc3c3cc(-n4c5ccccc5c5ccccc54)ccc32)cc1 ; c1ccc(-n2c3ccccc3c3cc(-c4ccc5c(c4)c4cc(-n6c7ccccc7c7ccccc76)ccc4n5-c4ccccc4)ccc32)cc1 ; c1ccc(-n2c3ccccc3c3c(-c4cccc5c4c4cc(-n6c7ccccc7c7ccccc76)ccc4n5-c4ccccc4)cccc32)cc1]

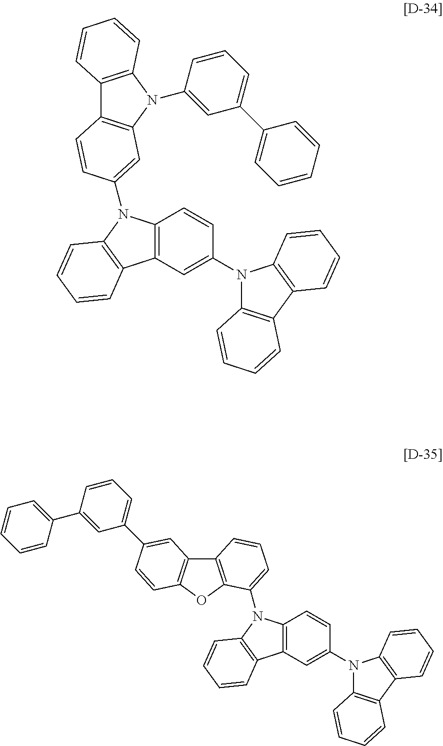 [SMILES: c1ccc(-c2cccc(-n3c4ccccc4c4ccc(-n5c6ccccc6c6cc(-n7c8ccccc8c8ccccc87)ccc65)cc43)c2)cc1 ; c1ccc(-c2cccc(-c3ccc4oc5c(-n6c7ccccc7c7cc(-n8c9ccccc9c9ccccc98)ccc76)cccc5c4c3)c2)cc1]

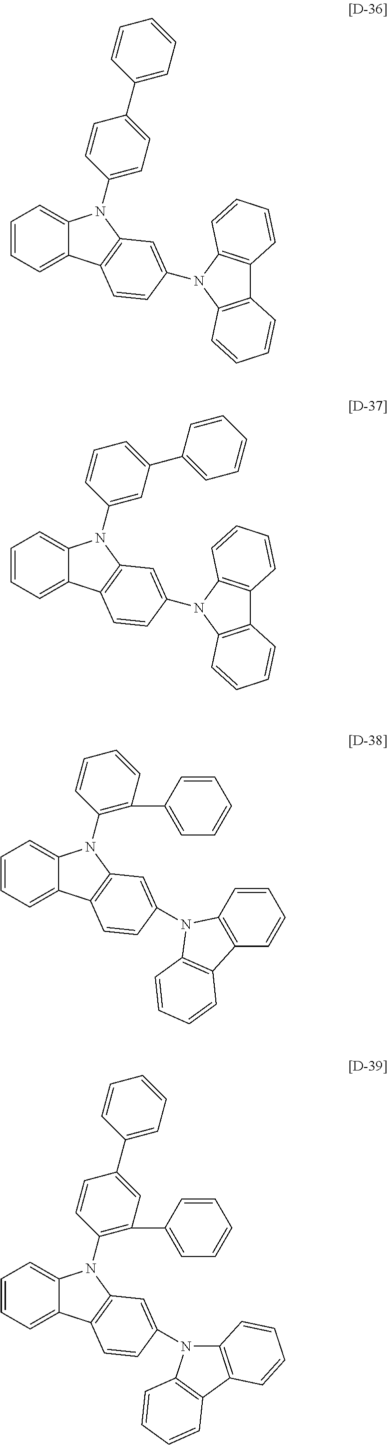 [SMILES: c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1 ; c1ccc(-c2ccccc2-n2c3ccccc3c3ccc(-n4c5ccccc5c5ccccc54)cc32)cc1 ; c1ccc(-c2ccc(-n3c4ccccc4c4ccc(-n5c6ccccc6c6ccccc65)cc43)c(-c3ccccc3)c2)cc1]

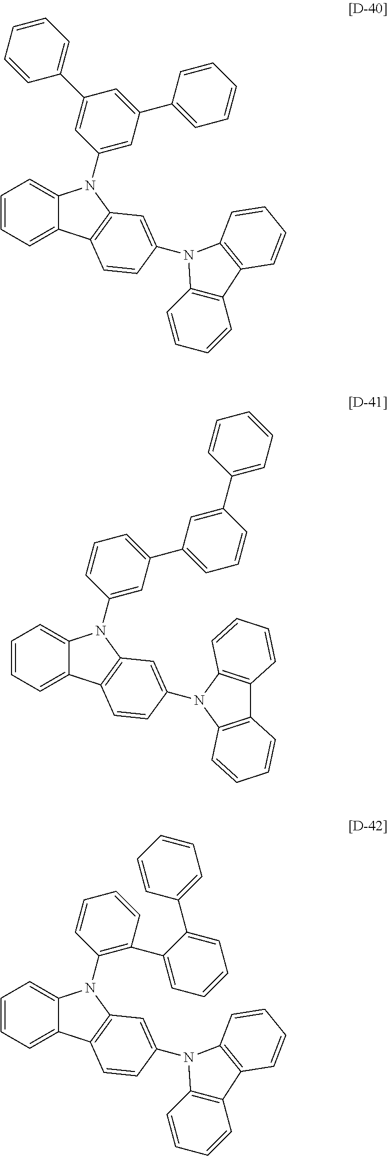 [SMILES: c1ccc(-c2cc(-c3ccccc3)cc(-n3c4ccccc4c4ccc(-n5c6ccccc6c6ccccc65)cc43)c2)cc1 ; c1ccc(-c2cccc(-c3cccc(-n4c5ccccc5c5ccc(-n6c7ccccc7c7ccccc76)cc54)c3)c2)cc1 ; c1ccc(-c2ccccc2-c2ccccc2-n2c3ccccc3c3ccc(-n4c5ccccc5c5ccccc54)cc32)cc1]

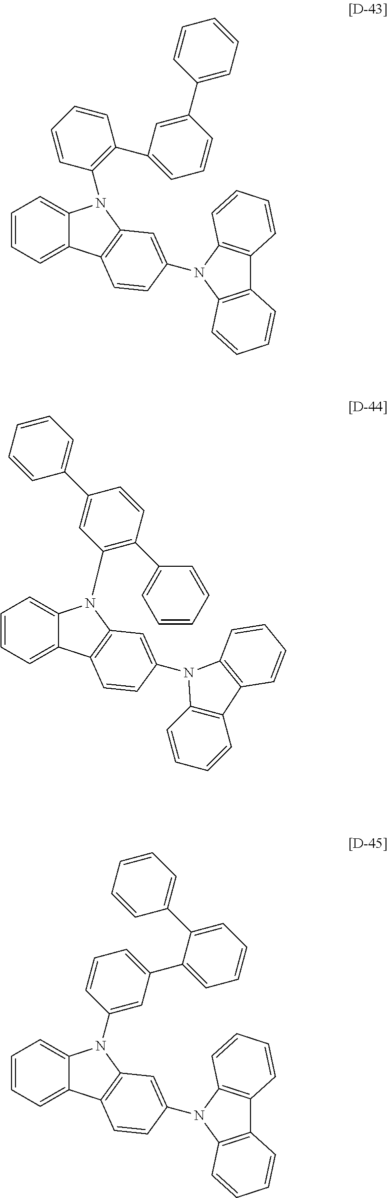 [SMILES: c1ccc(-c2cccc(-c3ccccc3-n3c4ccccc4c4ccc(-n5c6ccccc6c6ccccc65)cc43)c2)cc1 ; c1ccc(-c2ccc(-c3ccccc3)c(-n3c4ccccc4c4ccc(-n5c6ccccc6c6ccccc65)cc43)c2)cc1 ; c1ccc(-c2ccccc2-c2cccc(-n3c4ccccc4c4ccc(-n5c6ccccc6c6ccccc65)cc43)c2)cc1]

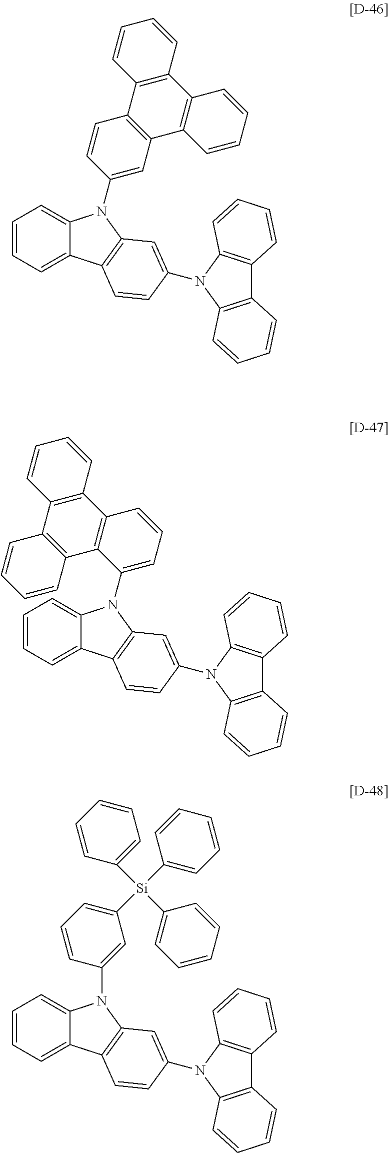 [SMILES: c1ccc2c(c1)c1ccccc1c1cc(-n3c4ccccc4c4ccc(-n5c6ccccc6c6ccccc65)cc43)ccc21 ; c1ccc2c(c1)c1ccccc1c1c(-n3c4ccccc4c4ccc(-n5c6ccccc6c6ccccc65)cc43)cccc21 ; c1ccc([Si](c2ccccc2)(c2ccccc2)c2cccc(-n3c4ccccc4c4ccc(-n5c6ccccc6c6ccccc65)cc43)c2)cc1]

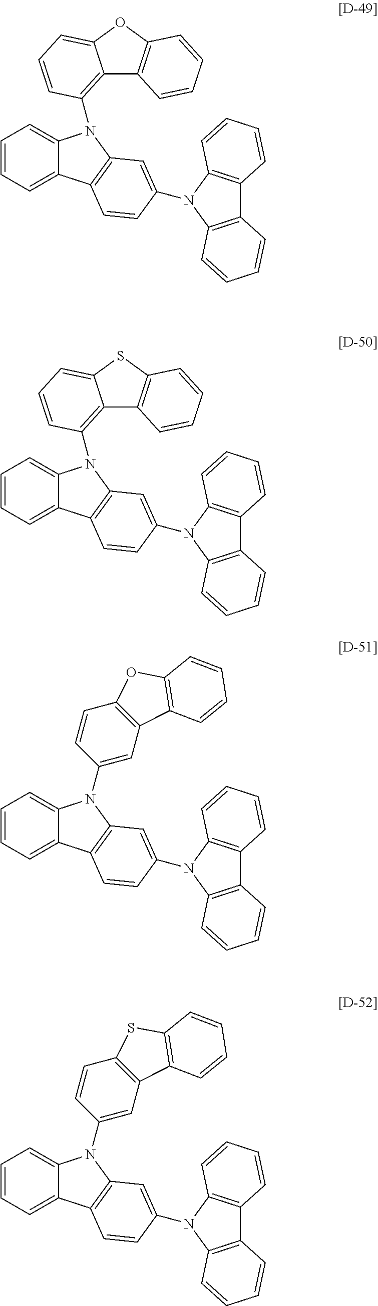 [SMILES: c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12 ; c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12]

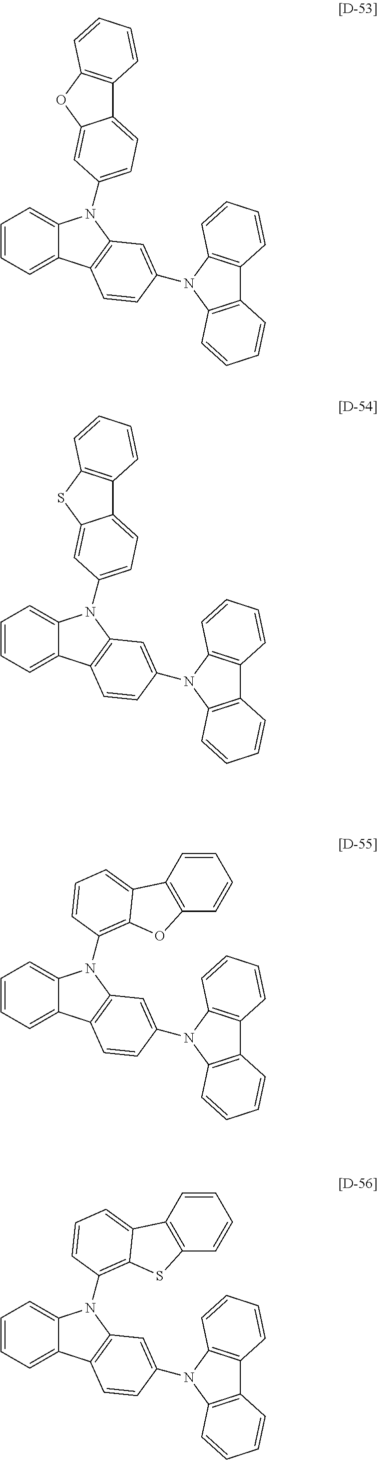 [SMILES: c1ccc2c(c1)oc1cc(-n3c4ccccc4c4ccc(-n5c6ccccc6c6ccccc65)cc43)ccc12 ; c1ccc2c(c1)sc1cc(-n3c4ccccc4c4ccc(-n5c6ccccc6c6ccccc65)cc43)ccc12 ; c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12]

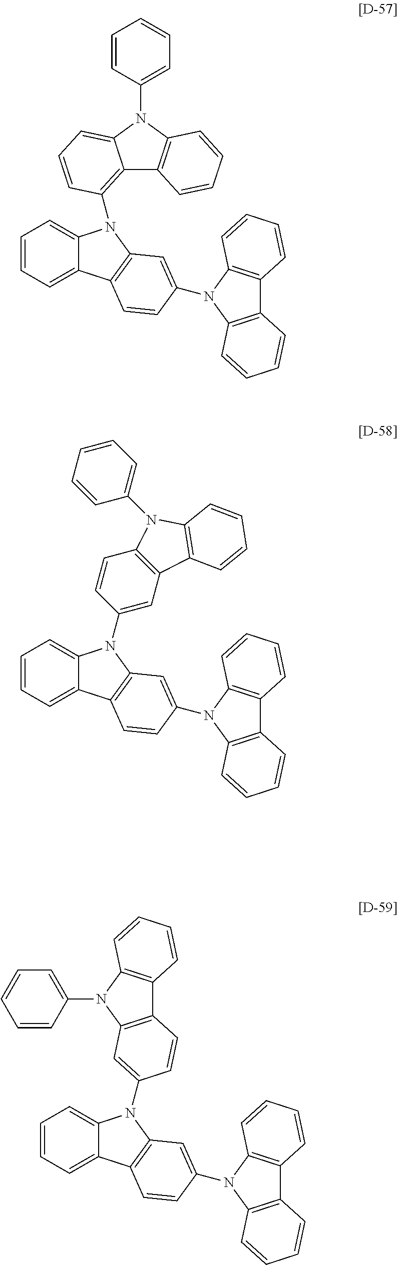 [SMILES: c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1 ; c1ccc(-n2c3ccccc3c3ccc(-n4c5ccccc5c5ccc(-n6c7ccccc7c7ccccc76)cc54)cc32)cc1]

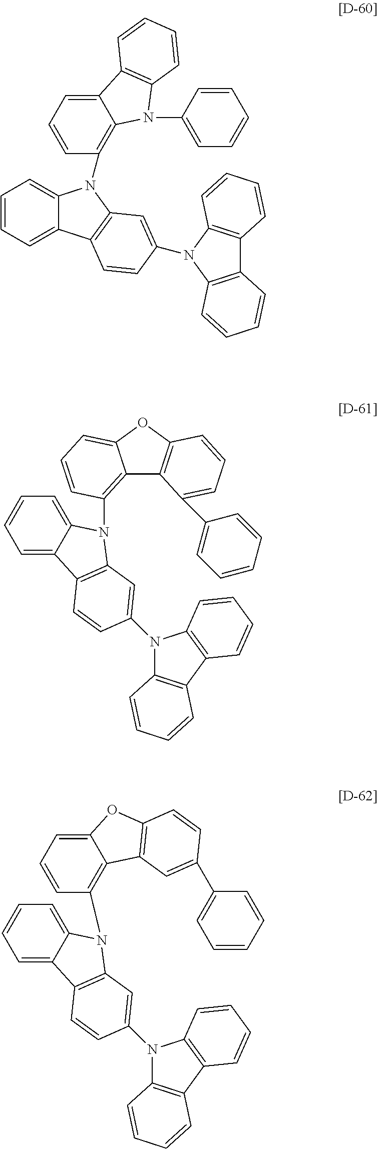 [SMILES: c1ccc(-n2c3ccccc3c3cccc(-n4c5ccccc5c5ccc(-n6c7ccccc7c7ccccc76)cc54)c32)cc1 ; c1ccc(-c2cccc3oc4cccc(-n5c6ccccc6c6ccc(-n7c8ccccc8c8ccccc87)cc65)c4c23)cc1 ; c1ccc(-c2ccc3oc4cccc(-n5c6ccccc6c6ccc(-n7c8ccccc8c8ccccc87)cc65)c4c3c2)cc1]

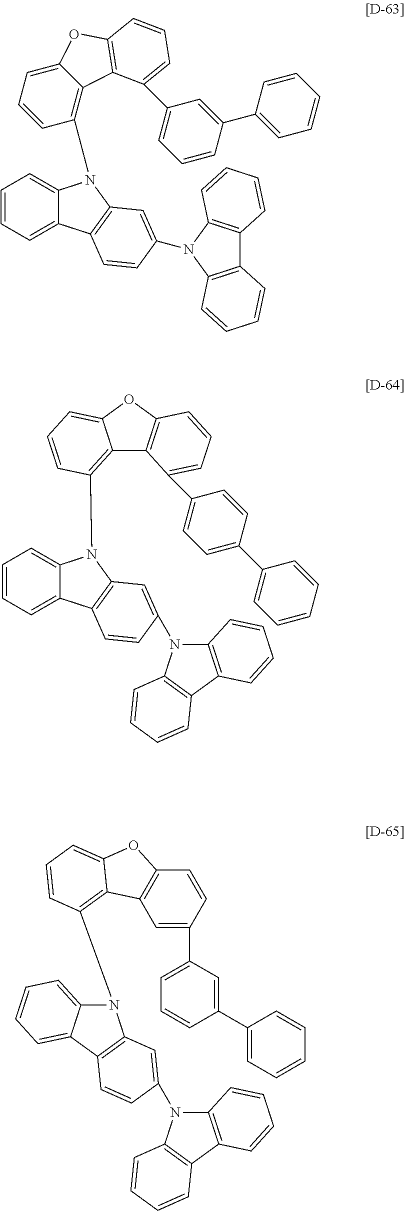 [SMILES: c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1 ; c1ccc(-c2cccc(-c3ccc4oc5cccc(-n6c7ccccc7c7ccc(-n8c9ccccc9c9ccccc98)cc76)c5c4c3)c2)cc1]

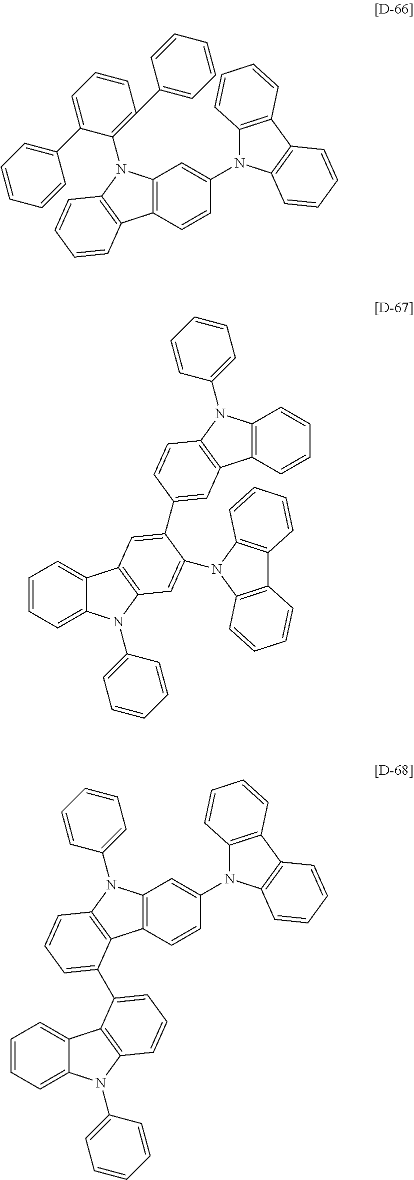 [SMILES: c1ccc(-c2cccc(-c3ccccc3)c2-n2c3ccccc3c3ccc(-n4c5ccccc5c5ccccc54)cc32)cc1 ; c1ccc(-n2c3ccccc3c3cc(-c4cc5c6ccccc6n(-c6ccccc6)c5cc4-n4c5ccccc5c5ccccc54)ccc32)cc1 ; c1ccc(-n2c3ccccc3c3c(-c4cccc5c4c4ccc(-n6c7ccccc7c7ccccc76)cc4n5-c4ccccc4)cccc32)cc1]

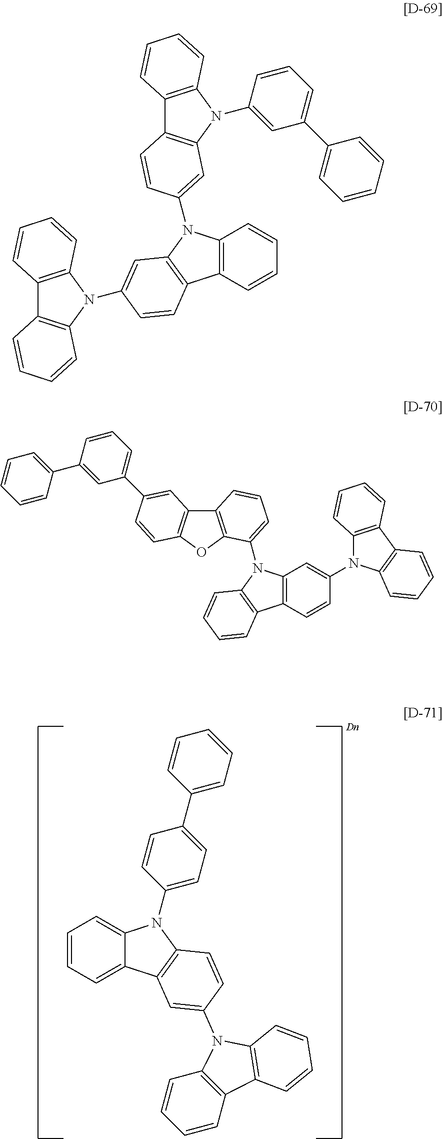

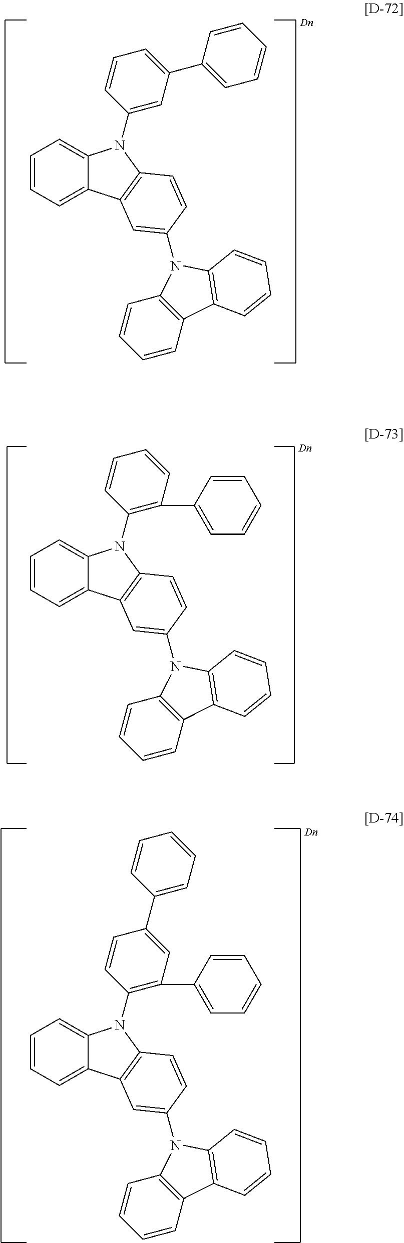

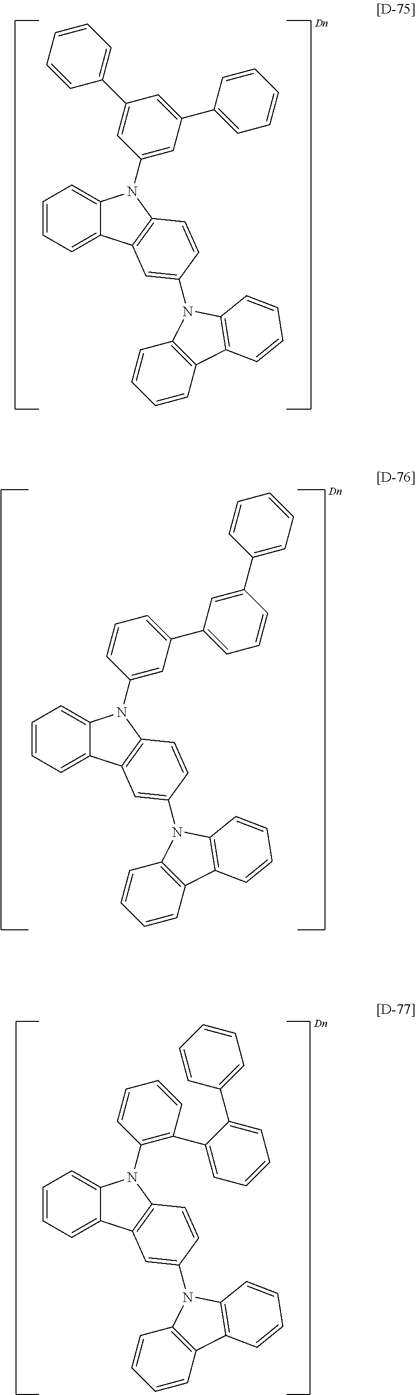

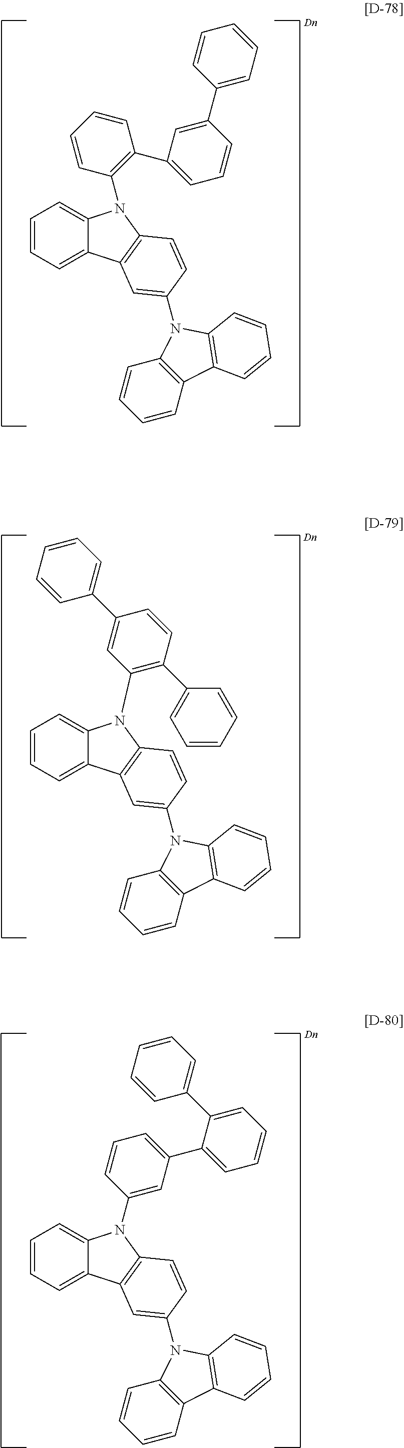

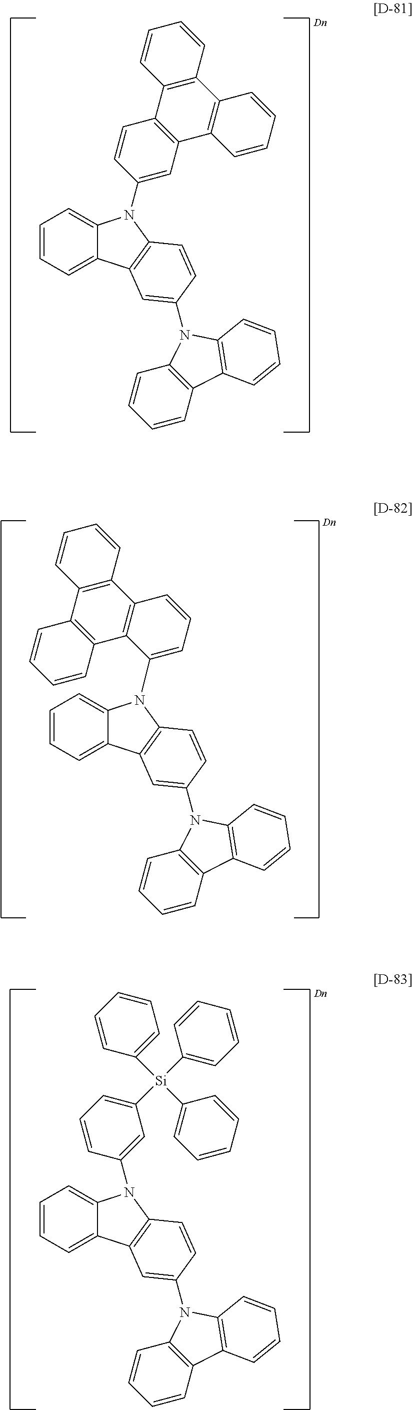

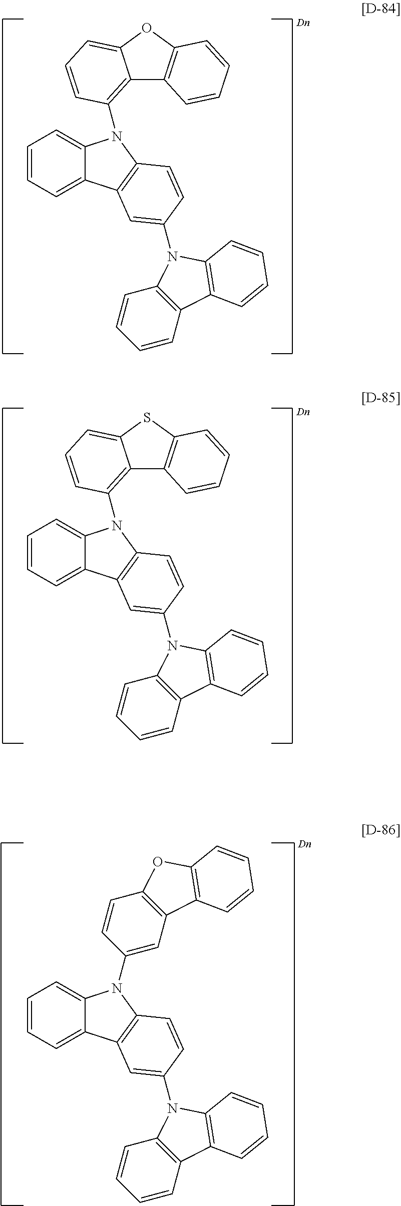

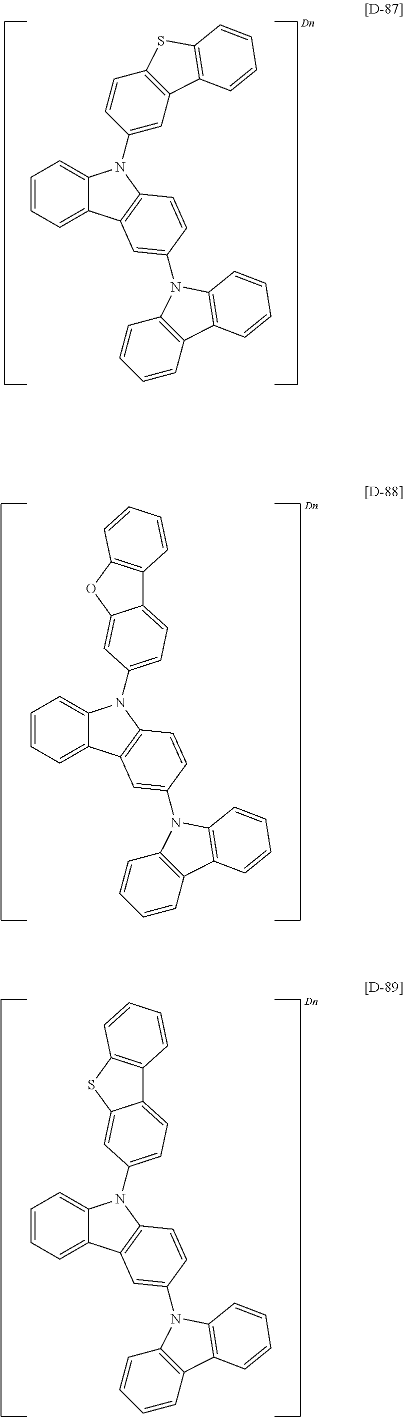

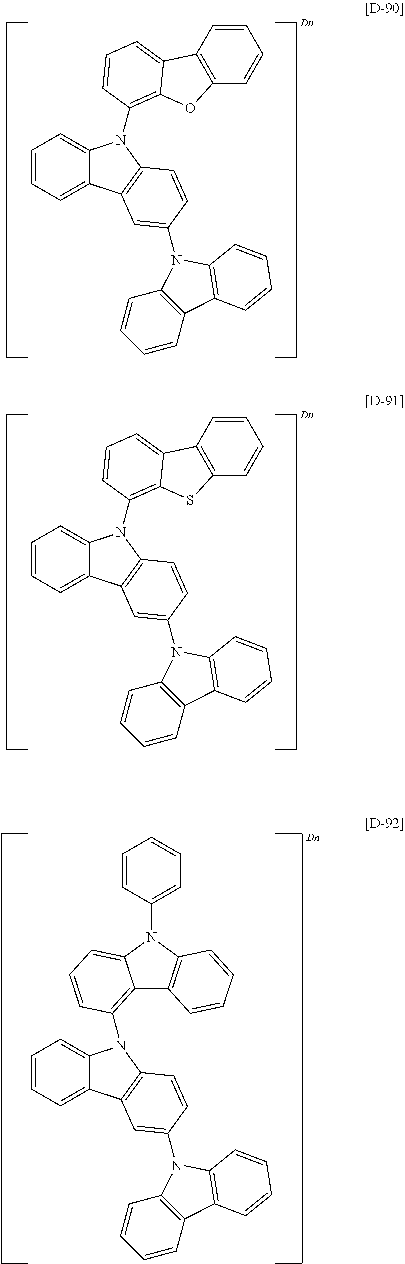

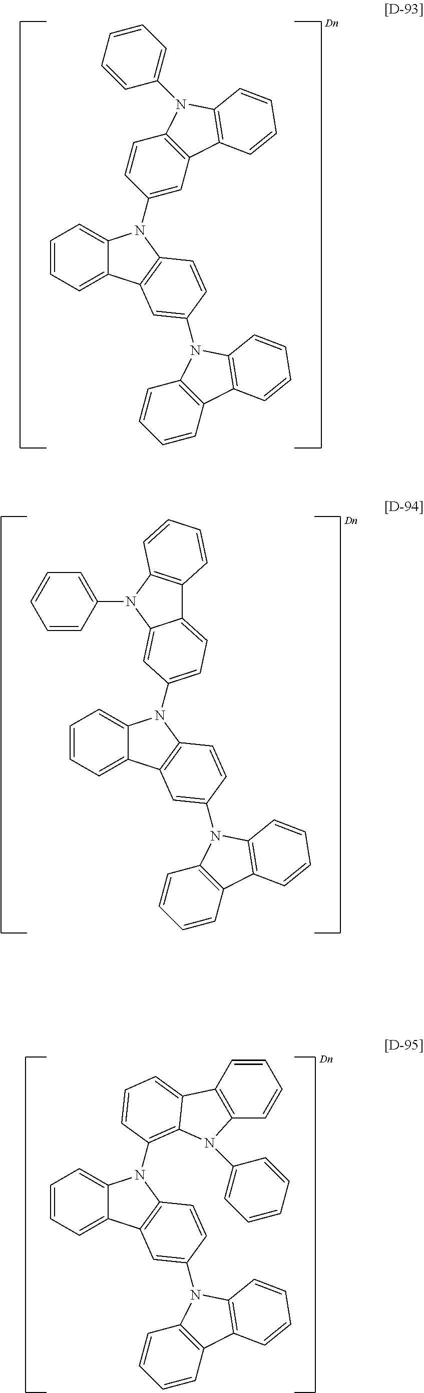

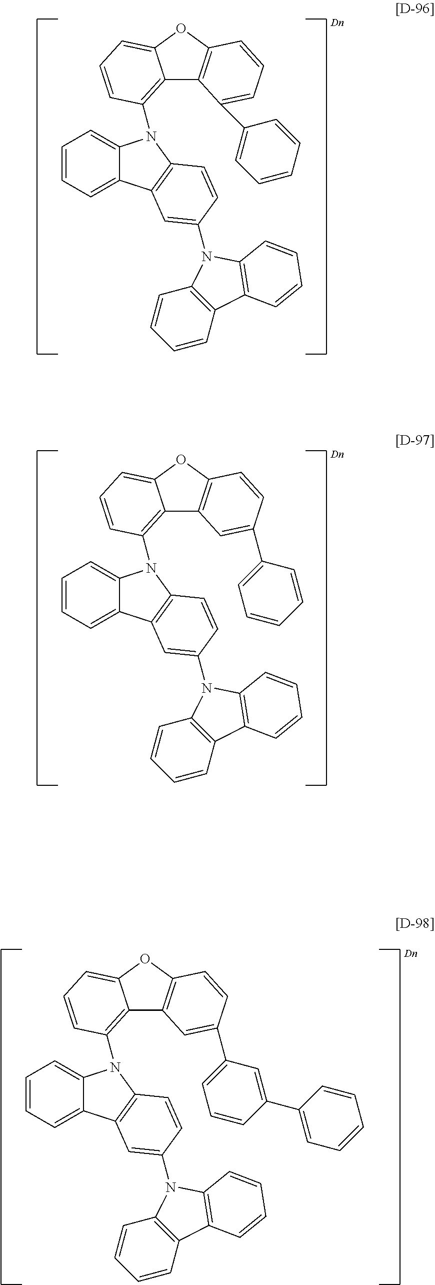

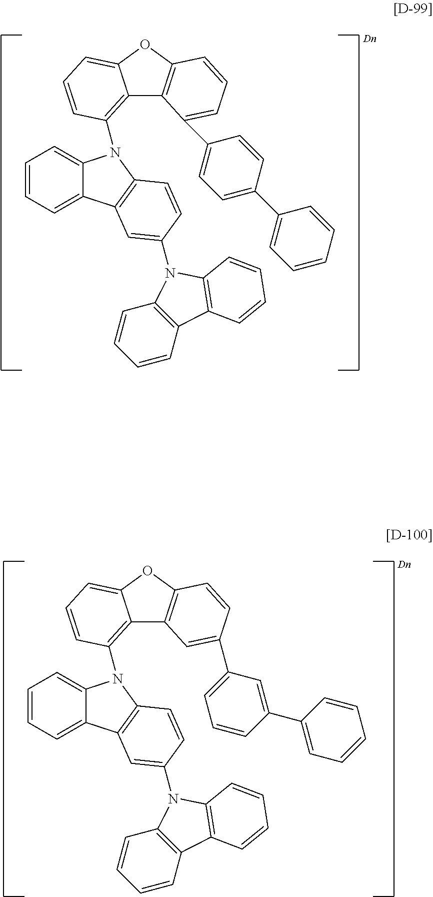

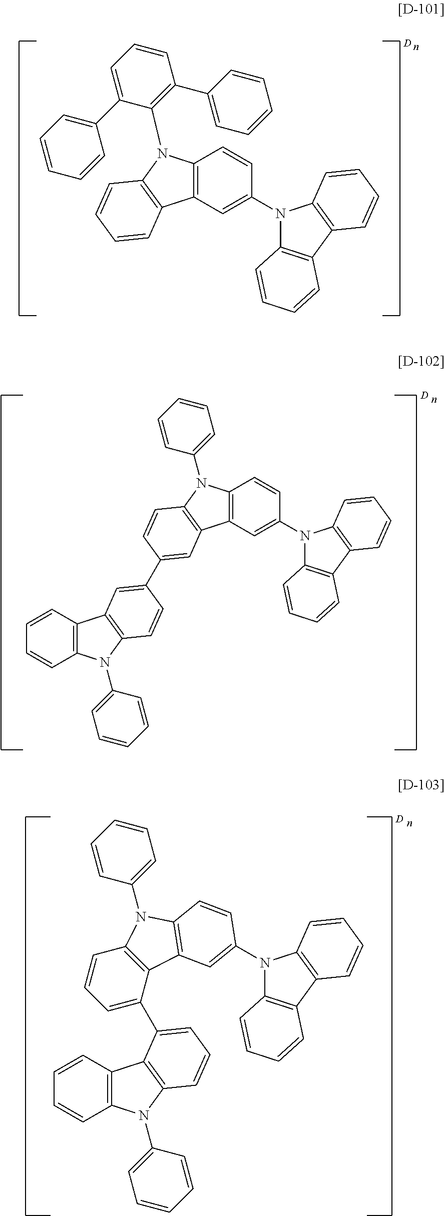

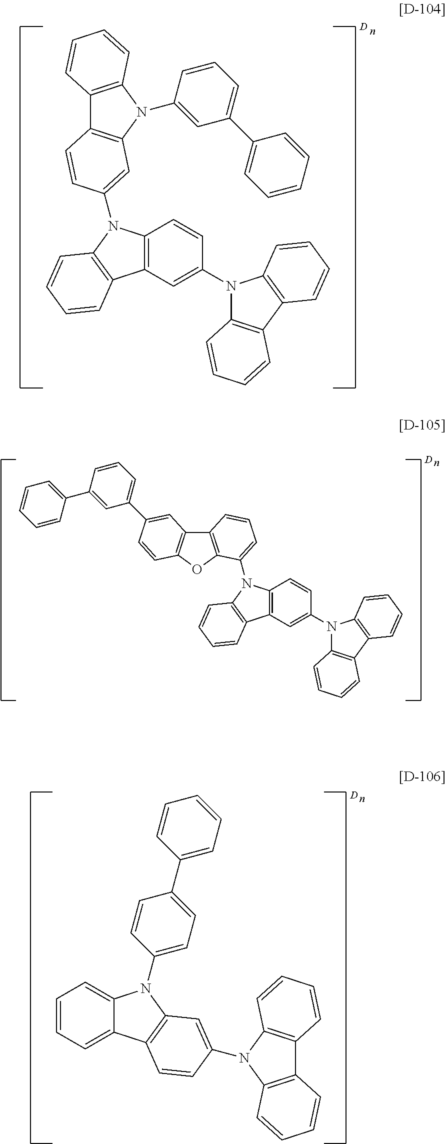

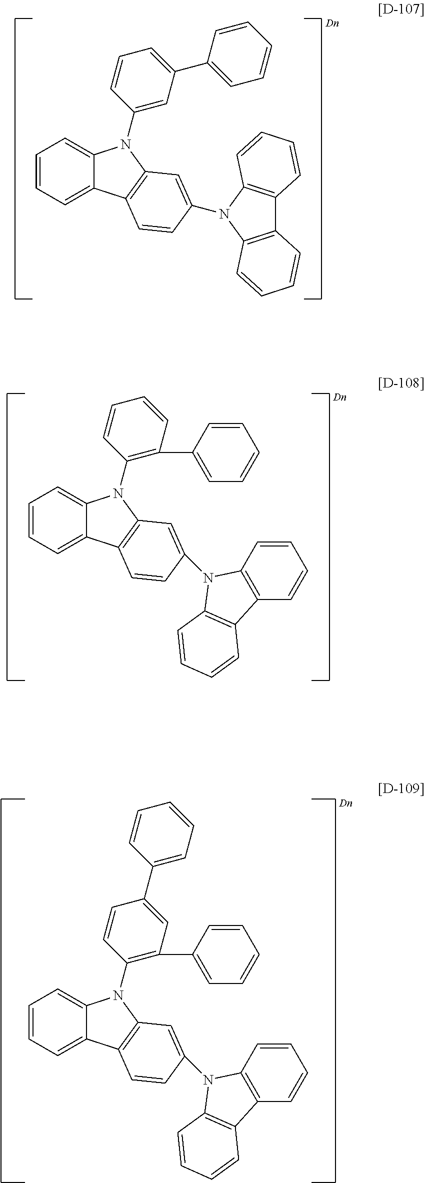

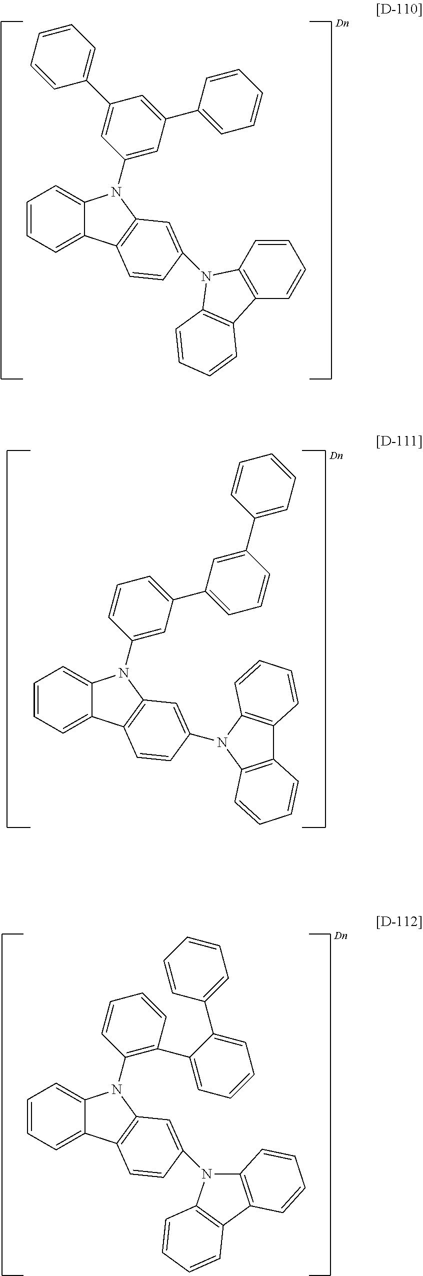

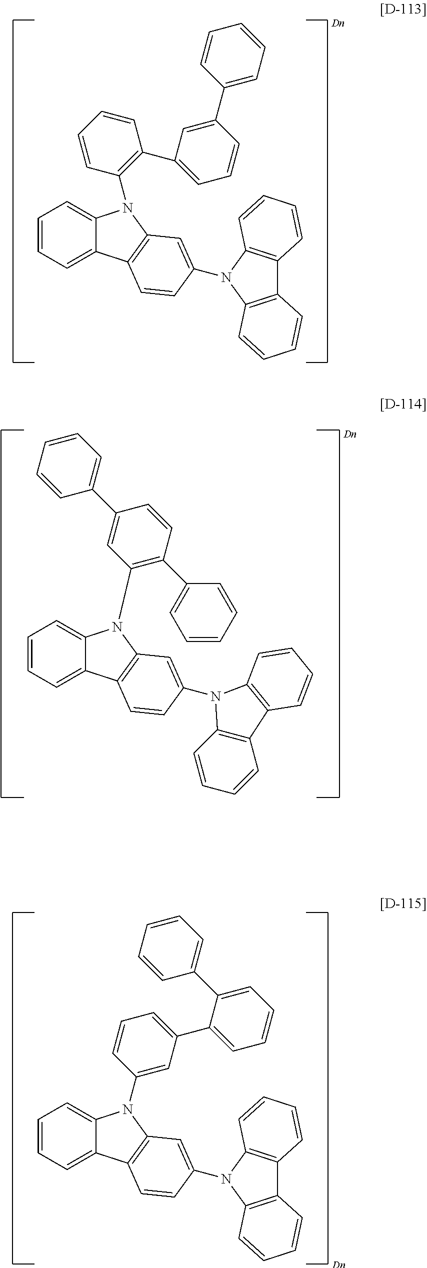

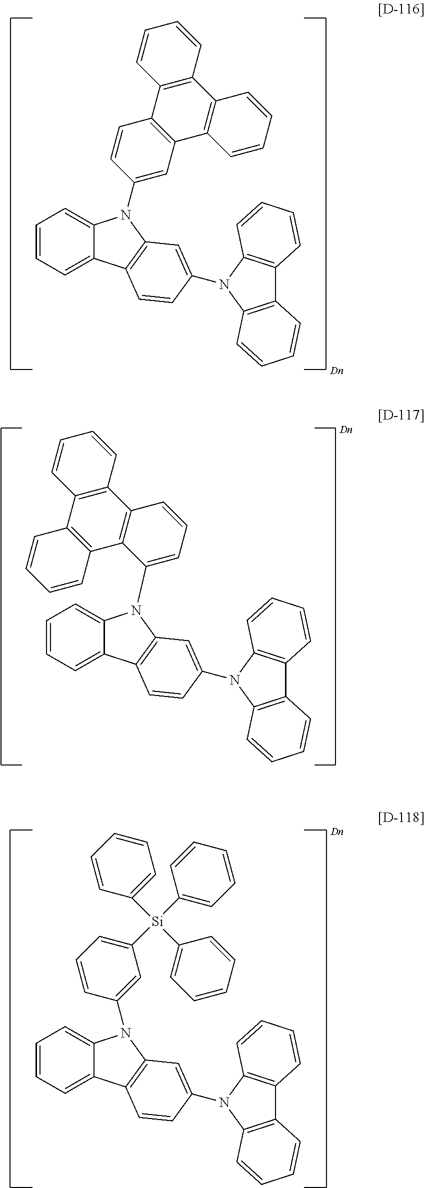

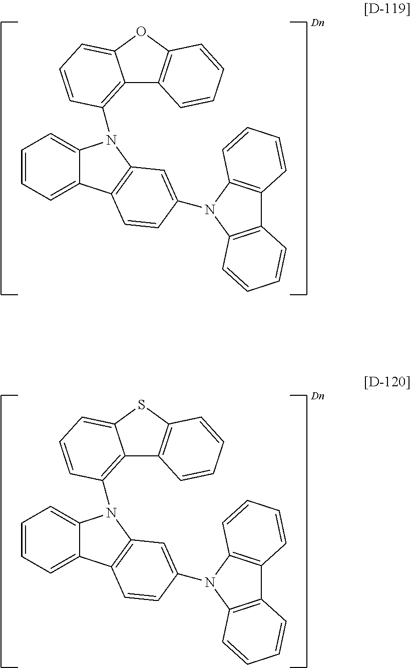

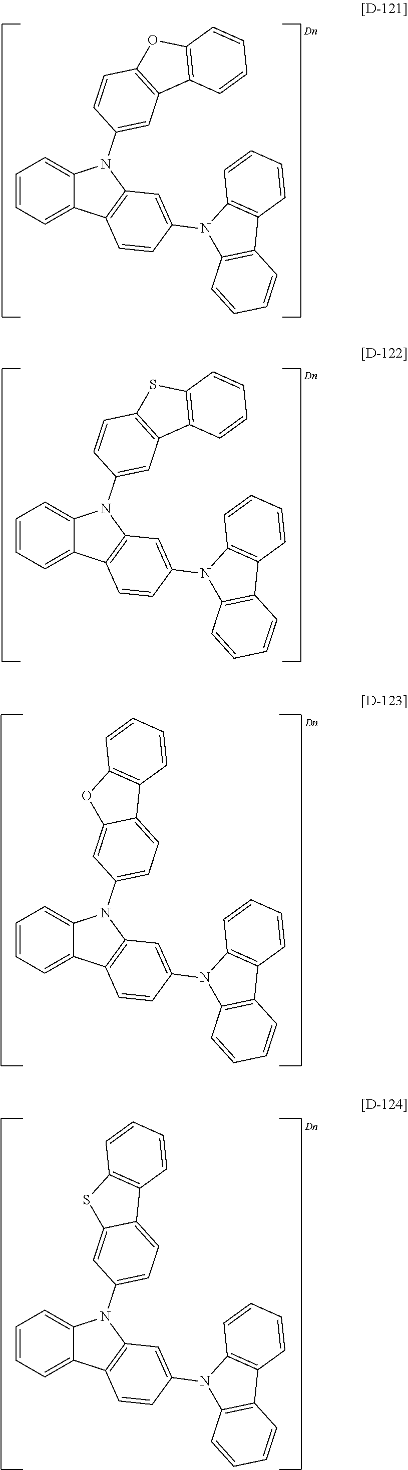

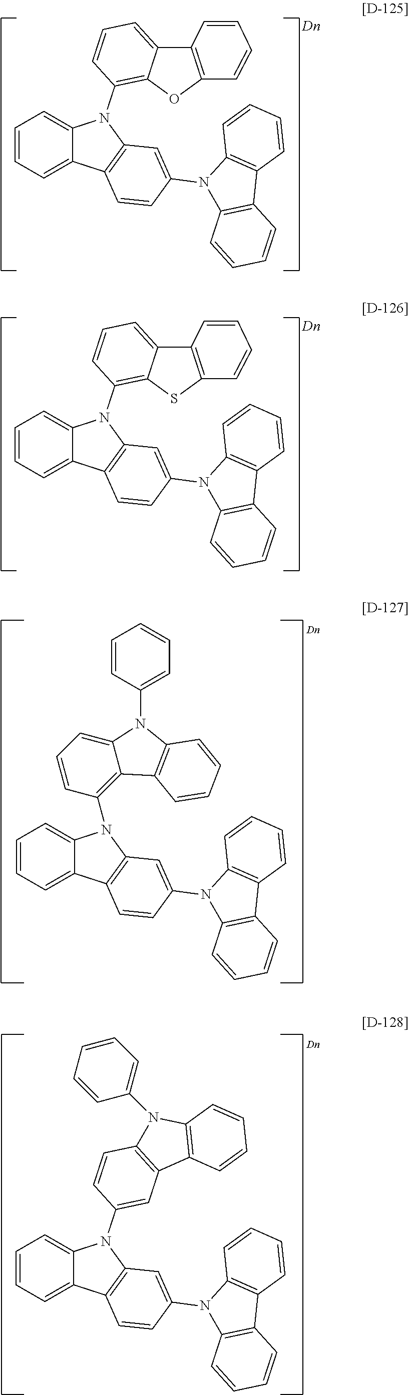

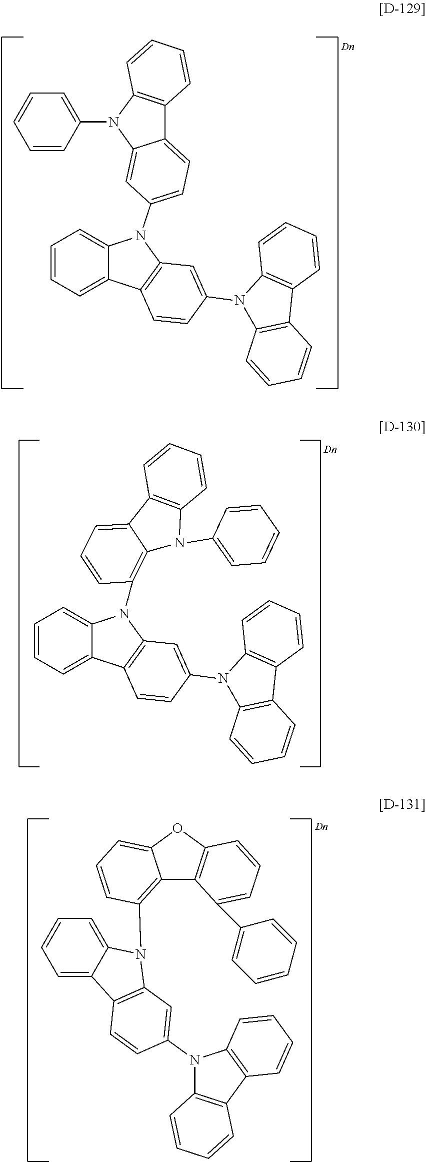

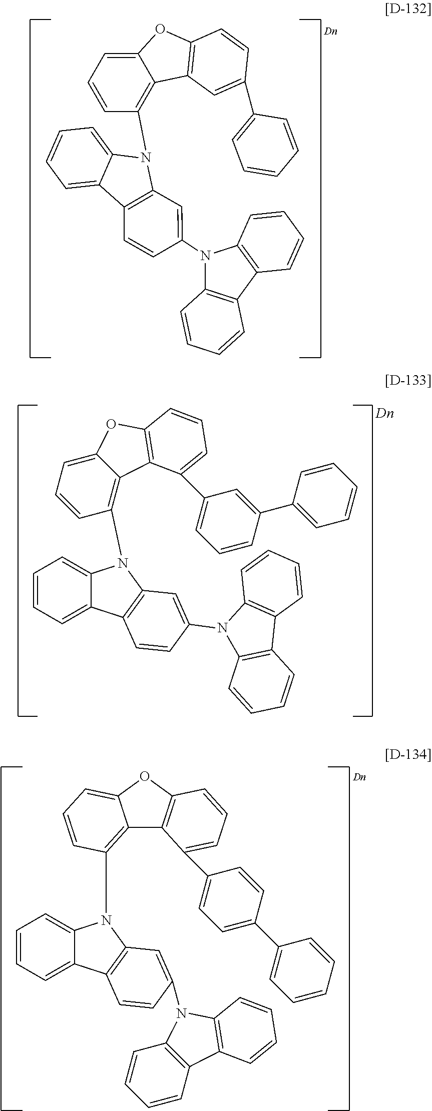

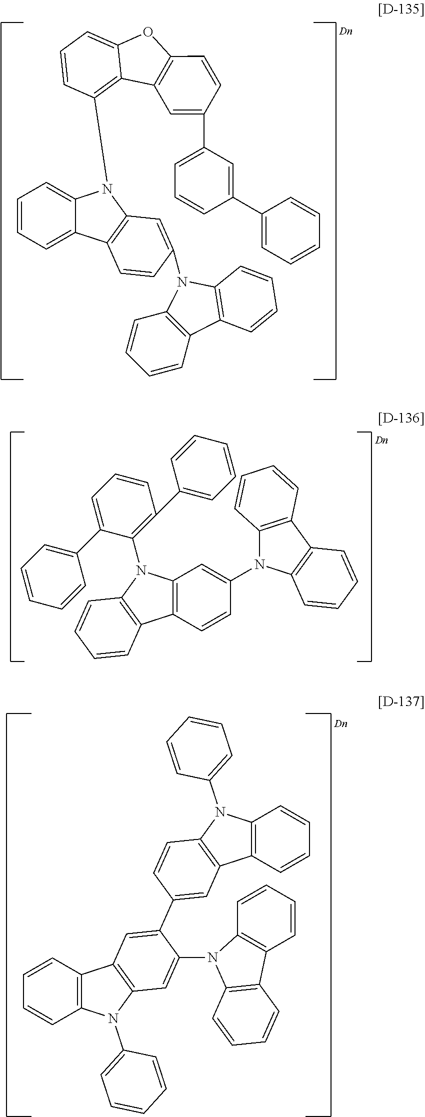

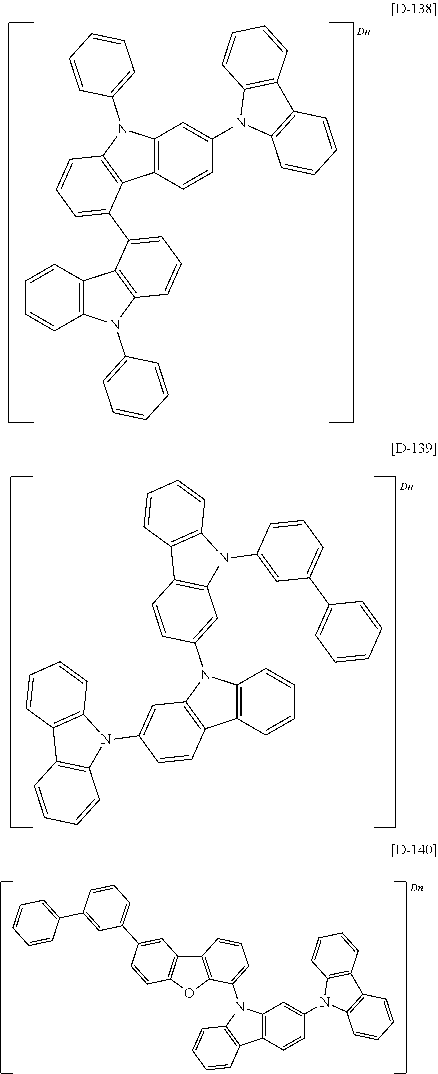

(Dn indicates the number of hydrogen atoms replaced by deuterium and indicates a structure in which one or more deuterium atoms are substituted.)

In an implementation, the second compound may be represented by Chemical Formula 3.

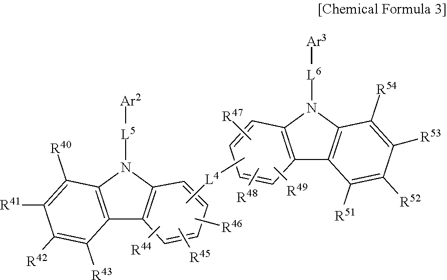

In Chemical Formula 3, L⁴ to L⁶ may each independently be or include, e.g., a single bond, a substituted or unsubstituted C6 to C30 arylene group, a substituted or unsubstituted divalent C3 to C30 heterocyclic group, or a combination thereof.

Ar² and Ar³ may each independently be or include, e.g., a substituted or unsubstituted C6 to C30 aryl group or a substituted or unsubstituted C2 to C30 heterocyclic group.

R⁴⁰ to R⁴⁹ and R⁵¹ to R⁵⁴ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

R⁴⁰ to R⁴⁹ and R⁵¹ to R⁵⁴ may each be separately present or two adjacent ones may be linked to each other to form a ring.

In an implementation, in Chemical Formula 3, L⁴ to L⁶ may each independently be, e.g., a single bond, a substituted or unsubstituted phenylene group, a substituted or unsubstituted biphenylene group, or a substituted or unsubstituted terphenylene group.

In an implementation, in Chemical Formula 3, Ar² and Ar³ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

In an implementation, in Chemical Formula 3, R⁴⁰ to R⁴⁹ and R⁵¹ to R⁵⁴ may each independently be, e.g., hydrogen, deuterium, a substituted or unsubstituted methyl group, a substituted or unsubstituted ethyl group, a substituted or unsubstituted phenyl group, a substituted or unsubstituted biphenyl group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, a substituted or unsubstituted fluorenyl group, a substituted or unsubstituted silyl group, or a cyano group.

In an implementation, in Chemical Formula 3, at least one of Ar², R⁴⁰ to R⁴⁹, or R⁵¹ to R⁵⁴ may be a substituted or unsubstituted carbazolyl group.

In an implementation, in Chemical Formula 3, at least one of the substituents may be substituted with deuterium. The number of substituted deuterium atoms may be 1 to the maximum number of hydrogens in the chemical formula, e.g., from 1 to 40 or from 1 to 30.

In an implementation, the second compound represented by Chemical Formula 3 may be represented by one of Chemical Formulas 3A to 3M.

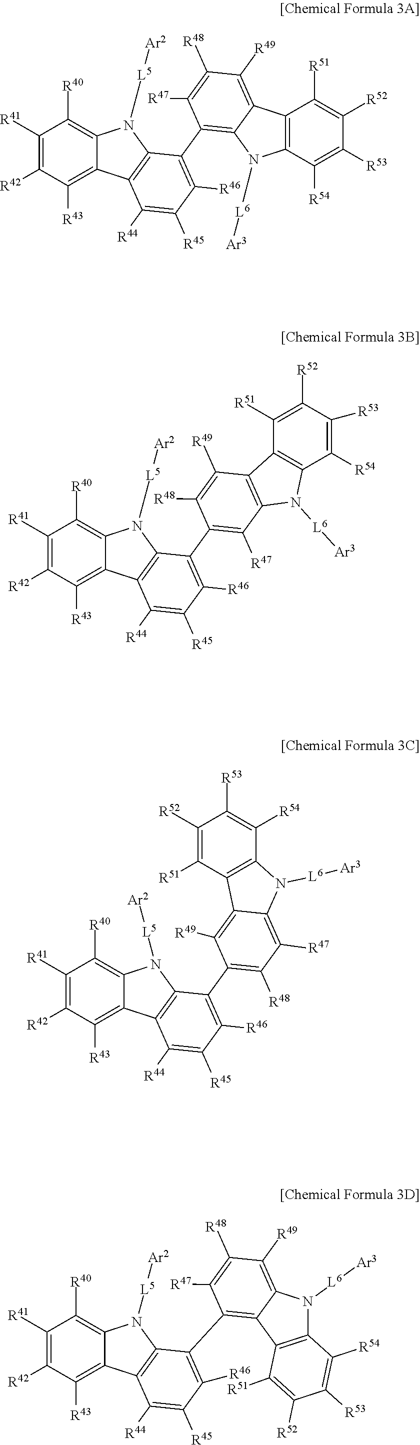

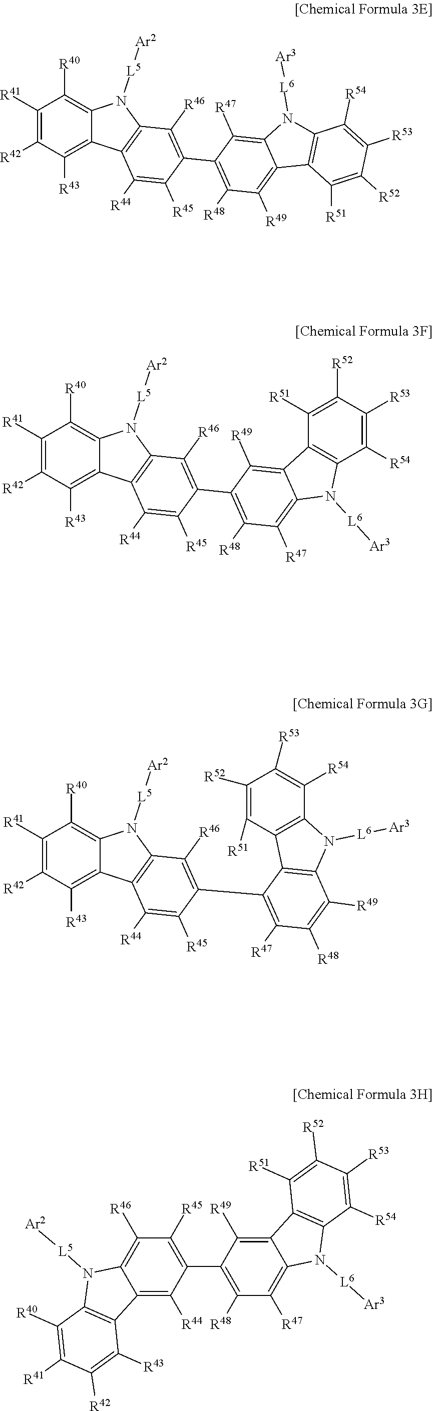

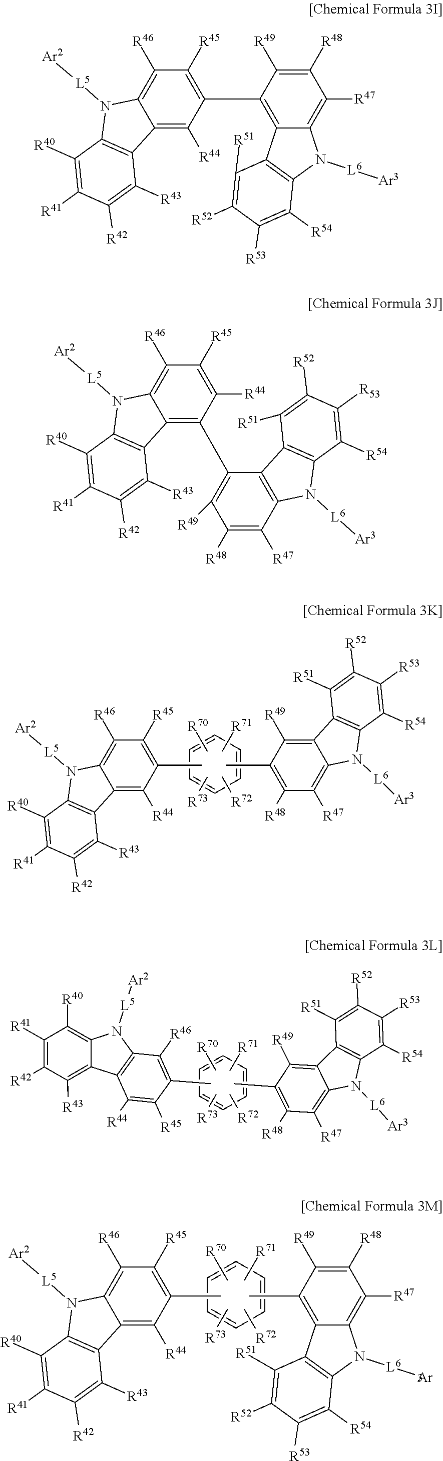

In Chemical Formulas 3A to 3M, L⁵, L⁶, Ar², Ar³, R⁴⁰ to R⁴⁹, and R⁵¹ to R⁵⁴ may be defined the same as those described above.

R⁷⁰ to R⁷³ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

R⁷⁰ to R⁷³ may each independently be separately present or two adjacent ones may be linked to each other to form a ring.

In an implementation, in Chemical Formulas 3 and 3A to 3M, L⁵ and L⁶ may each independently be, e.g., a single bond or a substituted or unsubstituted C6 to C30 arylene group, and may be, e.g., a single bond, a substituted or unsubstituted phenylene group, a substituted or unsubstituted biphenylene group, or a substituted or unsubstituted terphenylene group.

In an implementation, in Chemical Formulas 3 and 3A to 3N, Ar² and Ar³ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

In an implementation, the second compound represented by Chemical Formula 3 may include, e.g., a compound of Group 3.

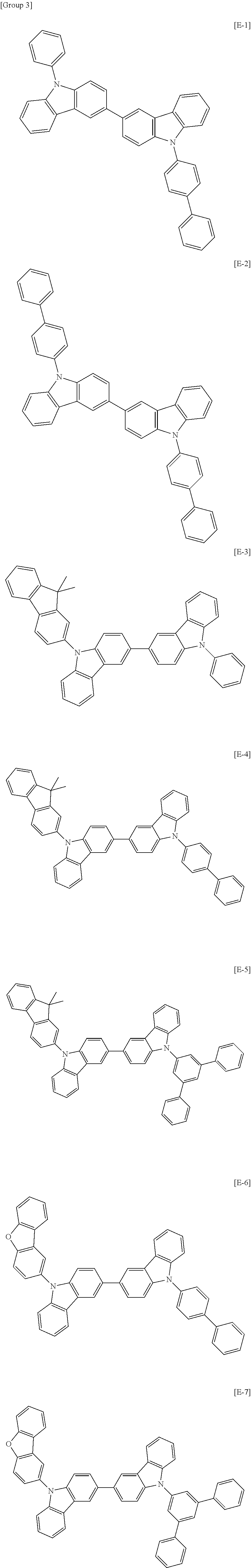

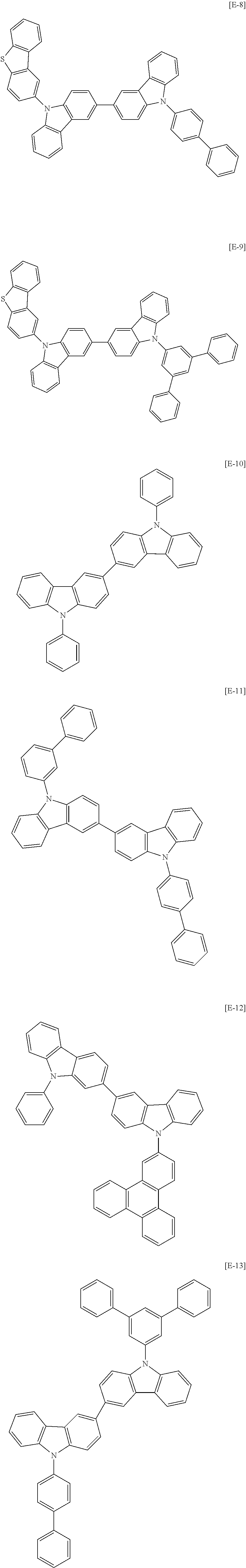

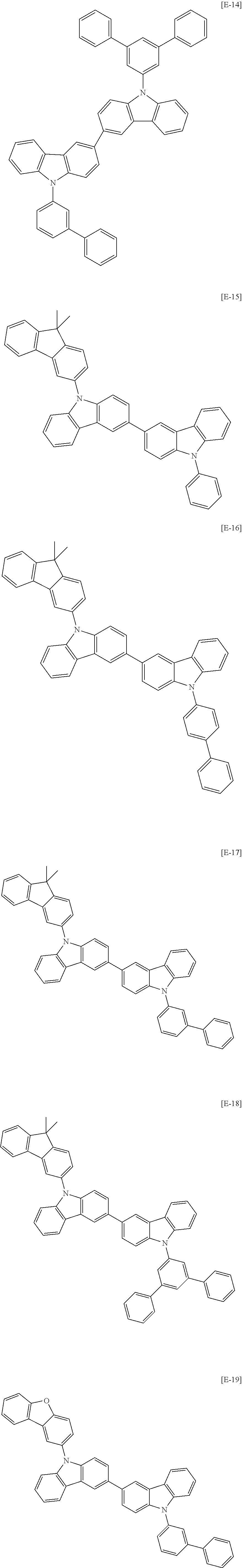

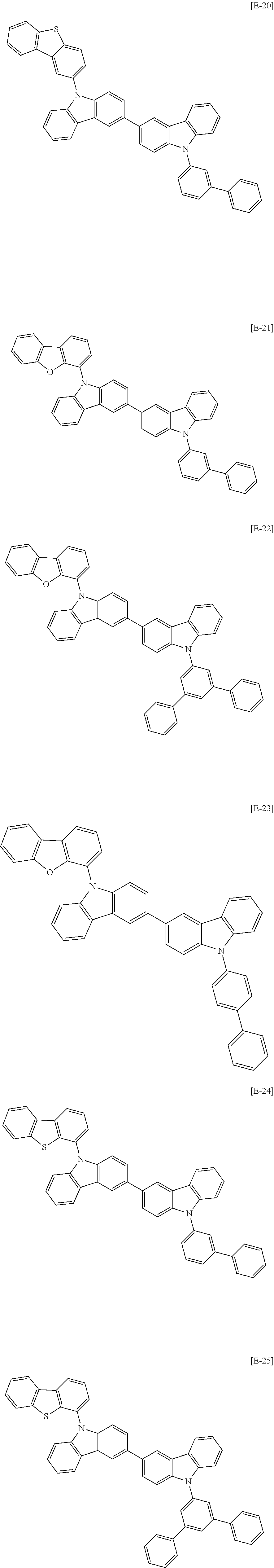

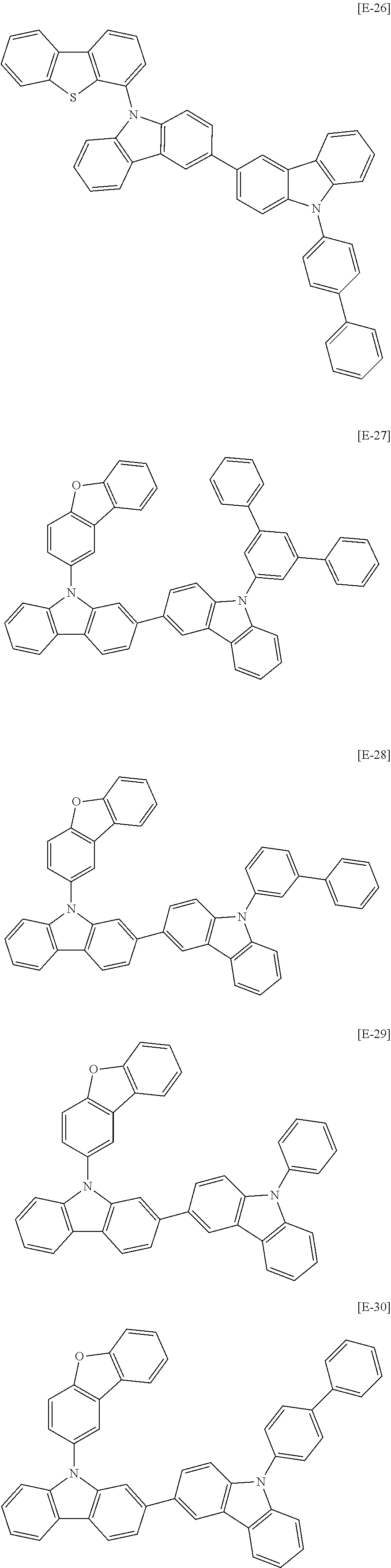 [SMILES: c1ccc(-c2ccc(-n3c4ccccc4c4cc(-c5ccc6c(c5)c5ccccc5n6-c5cccc6c5sc5ccccc56)ccc43)cc2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1 ; c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1]

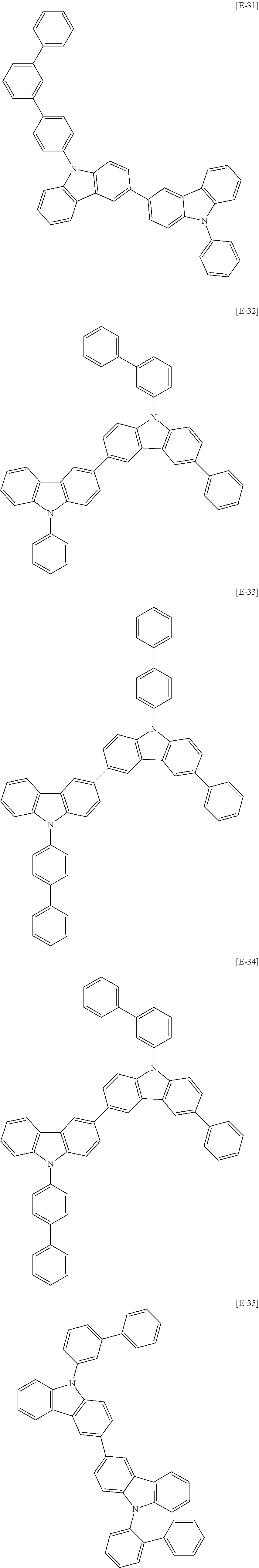 [SMILES: c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

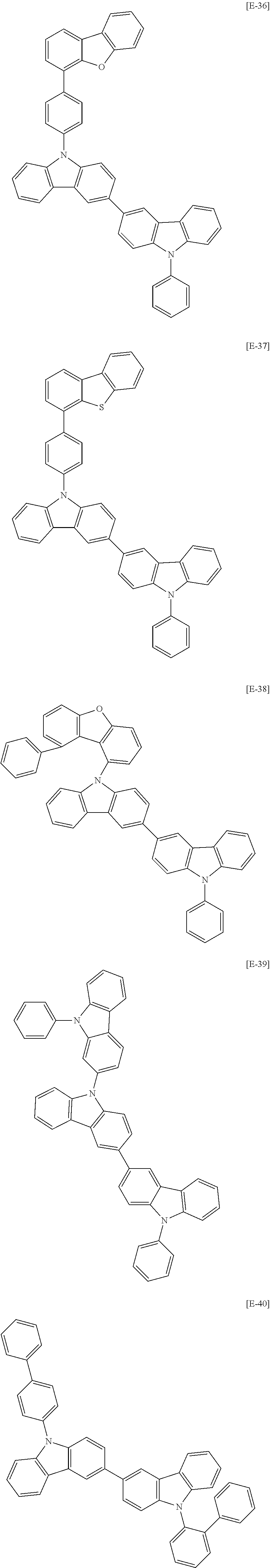 [SMILES: c1ccc(-n2c3ccccc3c3cc(-c4ccc5c(c4)c4ccccc4n5-c4ccc(-c5cccc6c5oc5ccccc56)cc4)ccc32)cc1 ; c1ccc(-n2c3ccccc3c3cc(-c4ccc5c(c4)c4ccccc4n5-c4ccc(-c5cccc6c5sc5ccccc56)cc4)ccc32)cc1 ; c1ccc(-c2cccc3oc4cccc(-n5c6ccccc6c6cc(-c7ccc8c(c7)c7ccccc7n8-c7ccccc7)ccc65)c4c23)cc1 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-c2ccc(-n3c4ccccc4c4cc(-c5ccc6c(c5)c5ccccc5n6-c5ccccc5-c5ccccc5)ccc43)cc2)cc1]

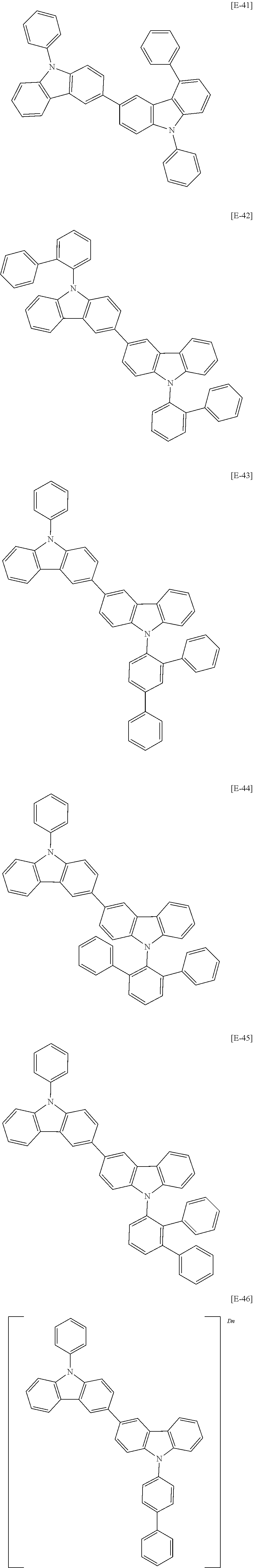

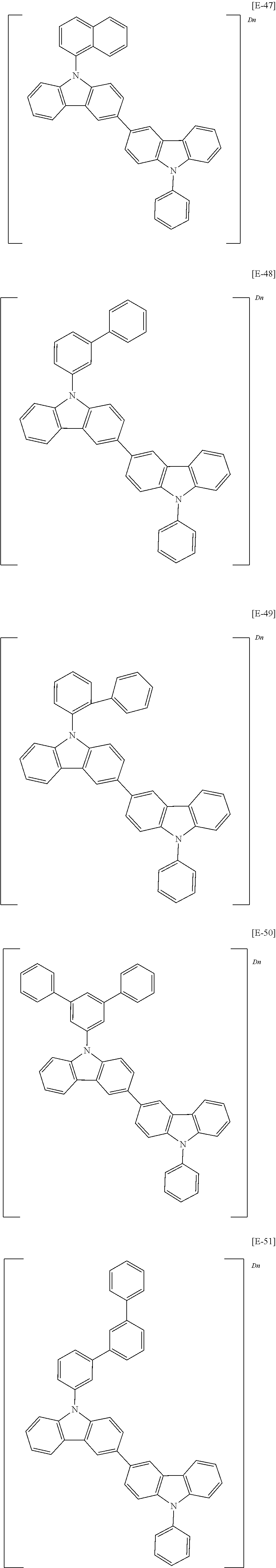

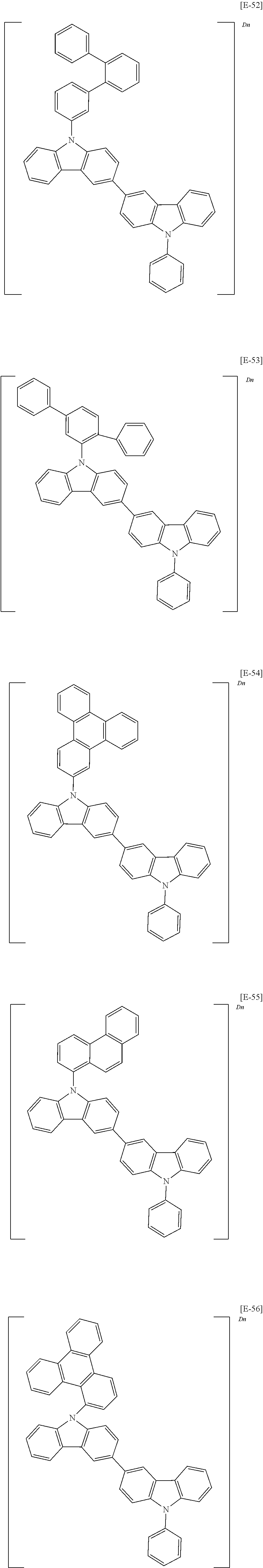

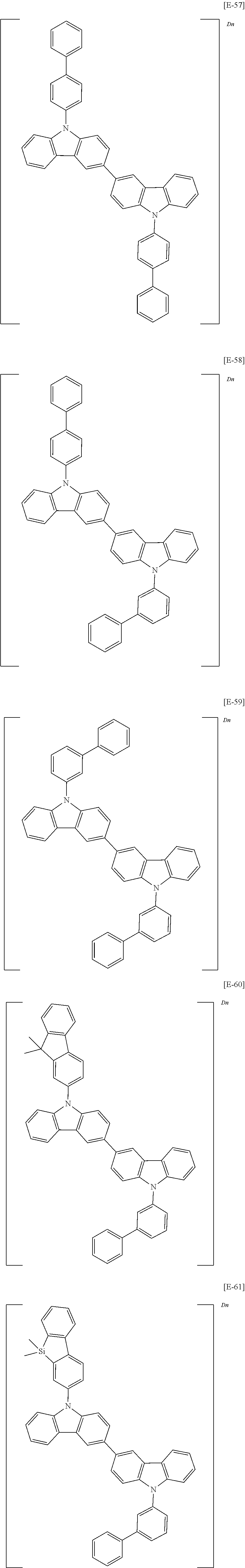

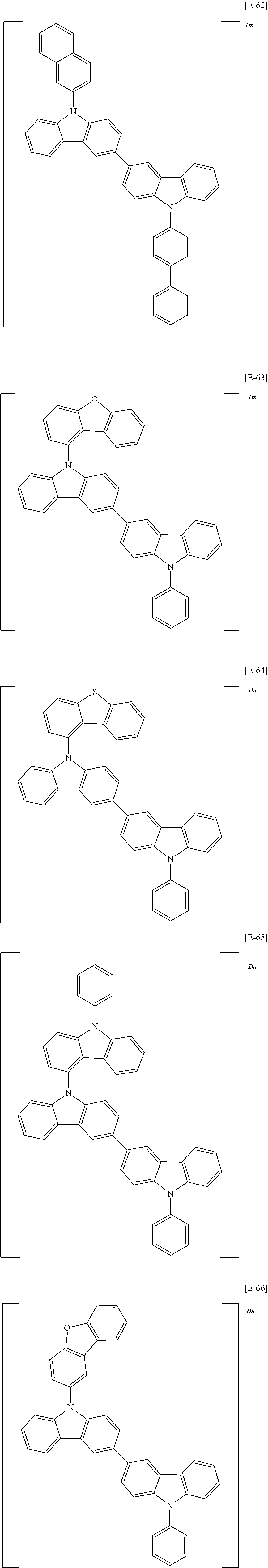

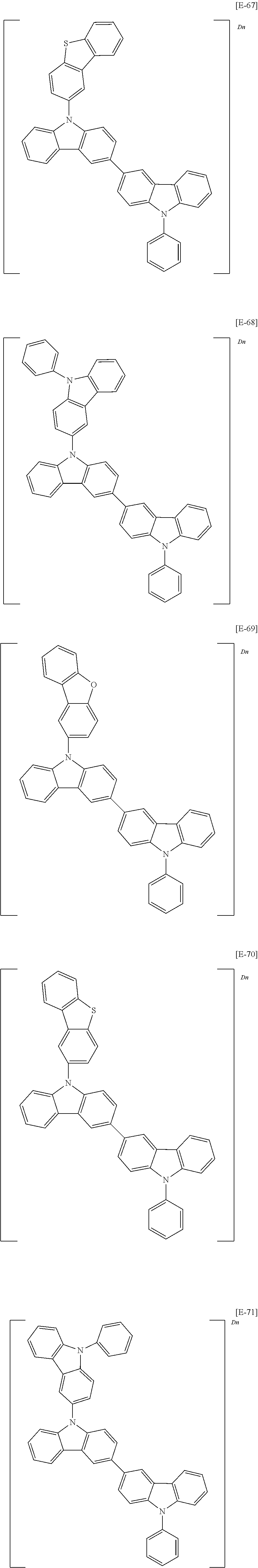

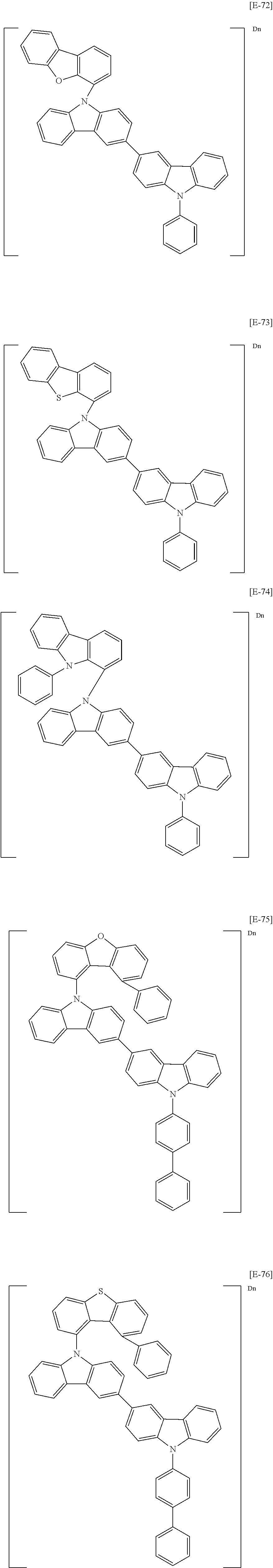

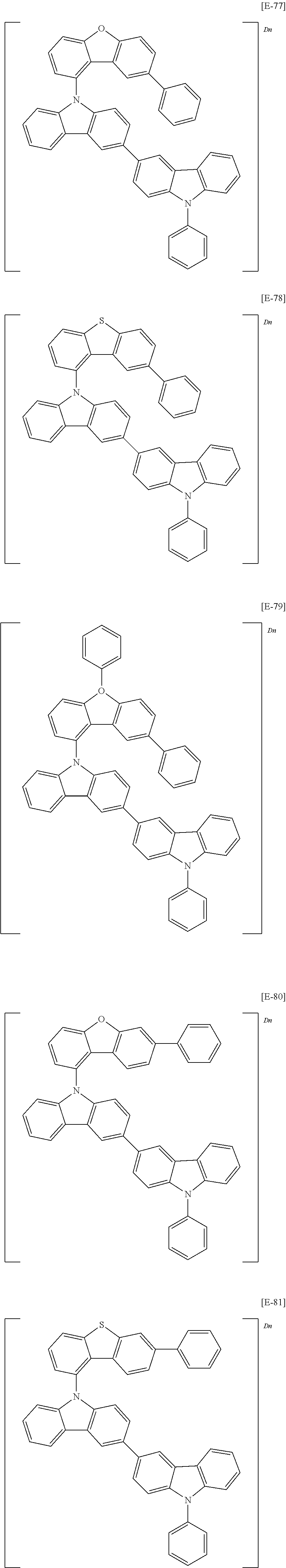

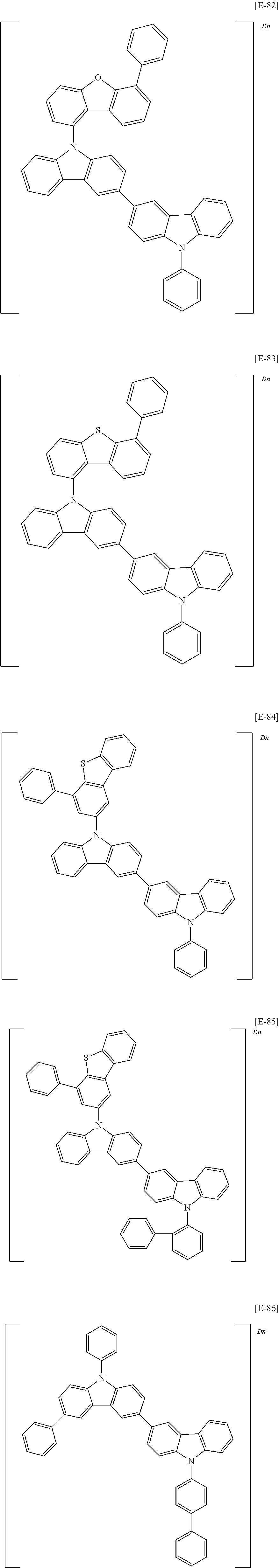

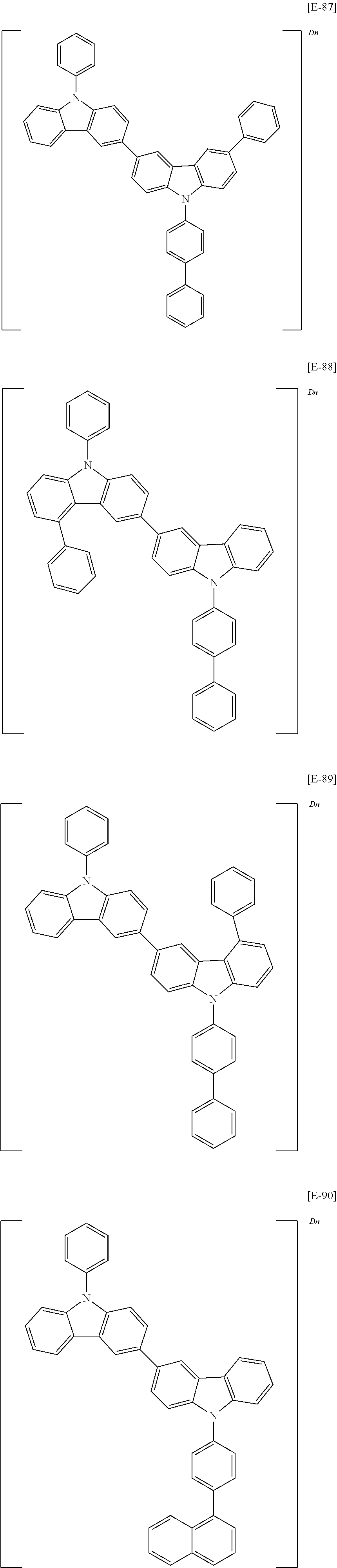

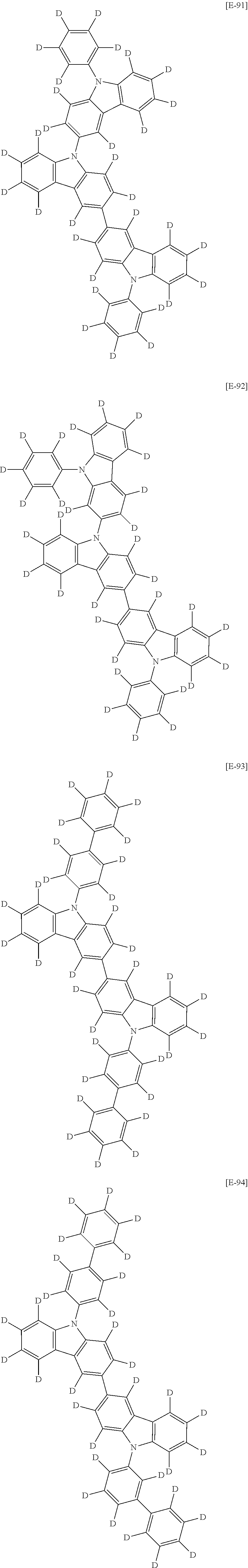

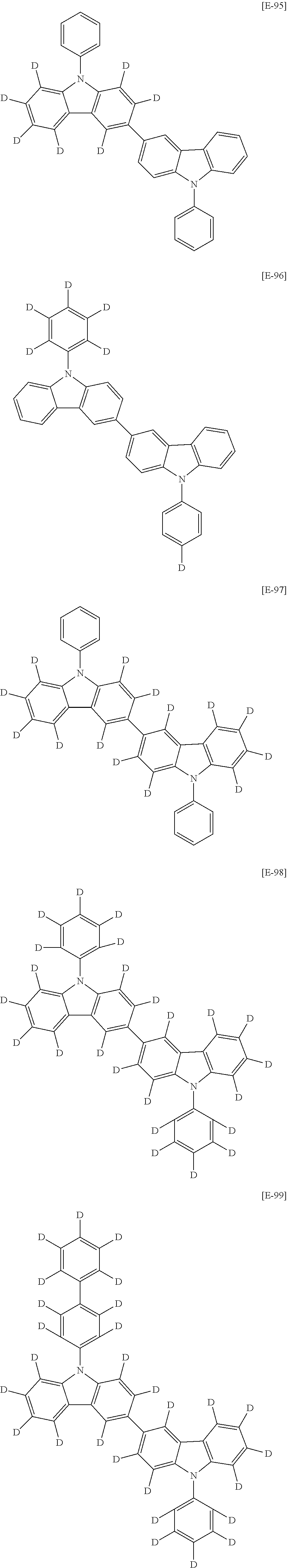

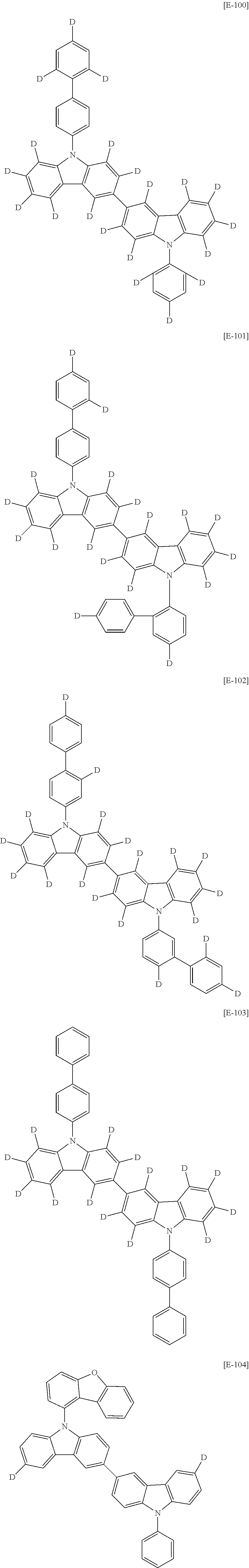

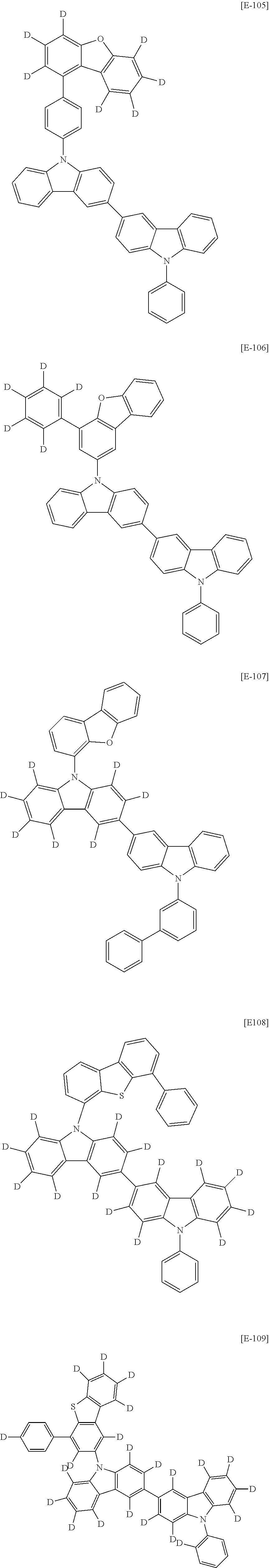

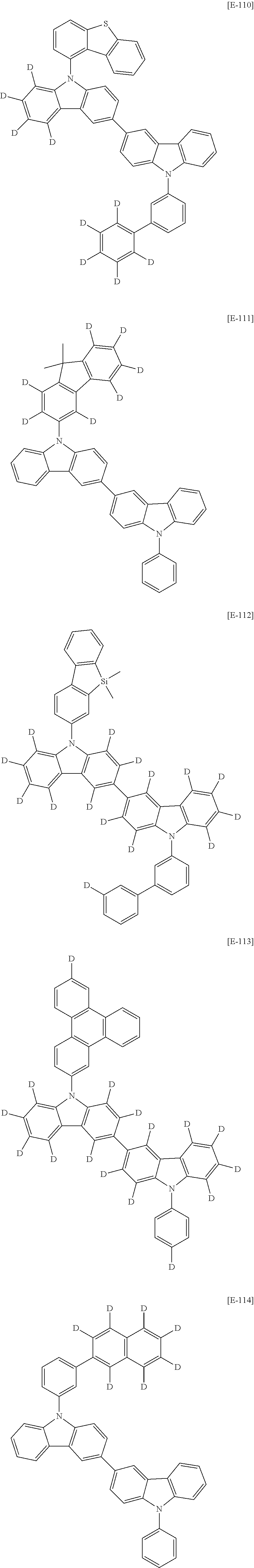

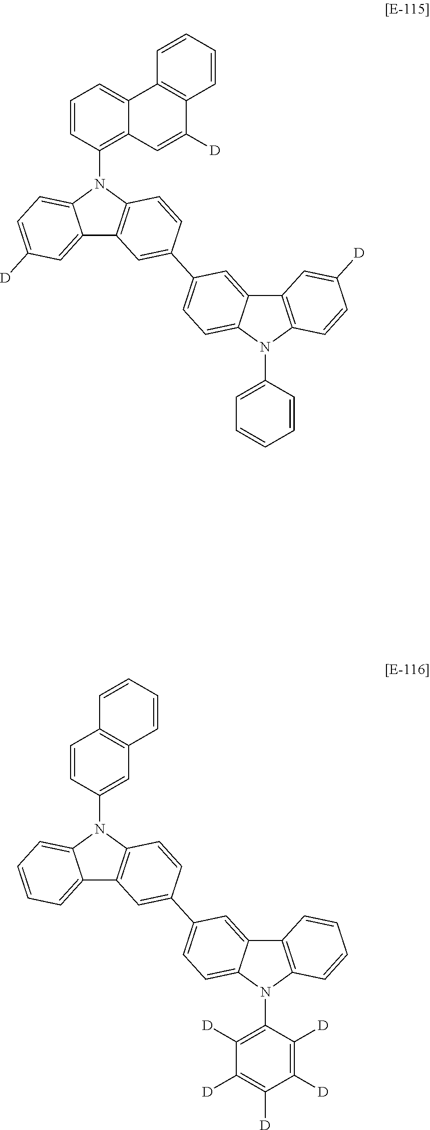 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H]]

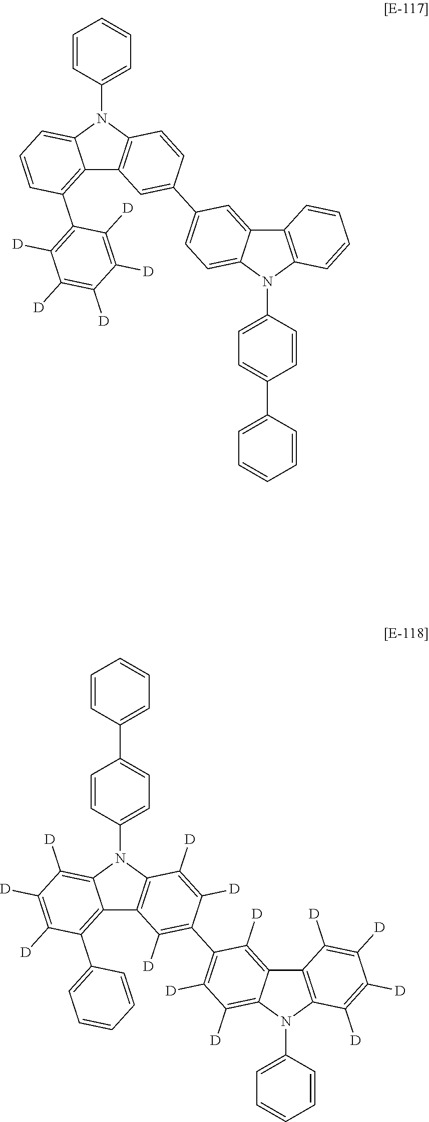 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H] ; [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

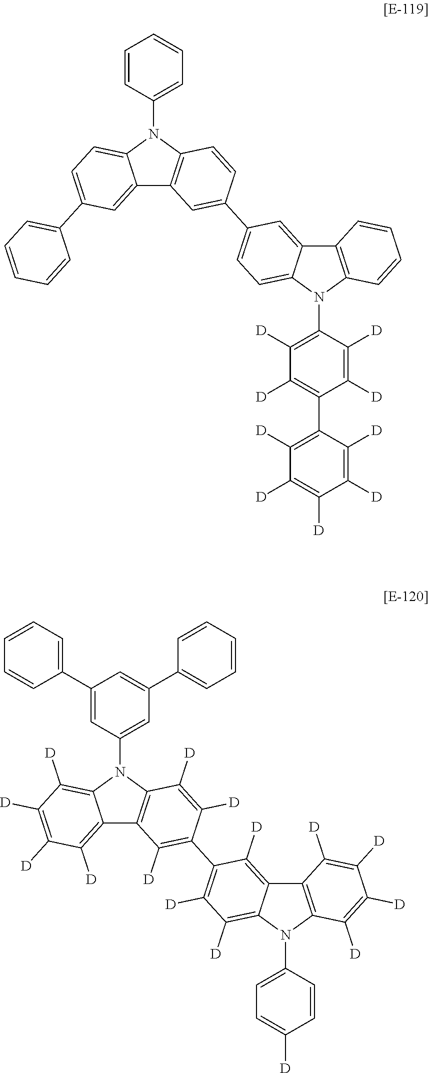 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H] ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

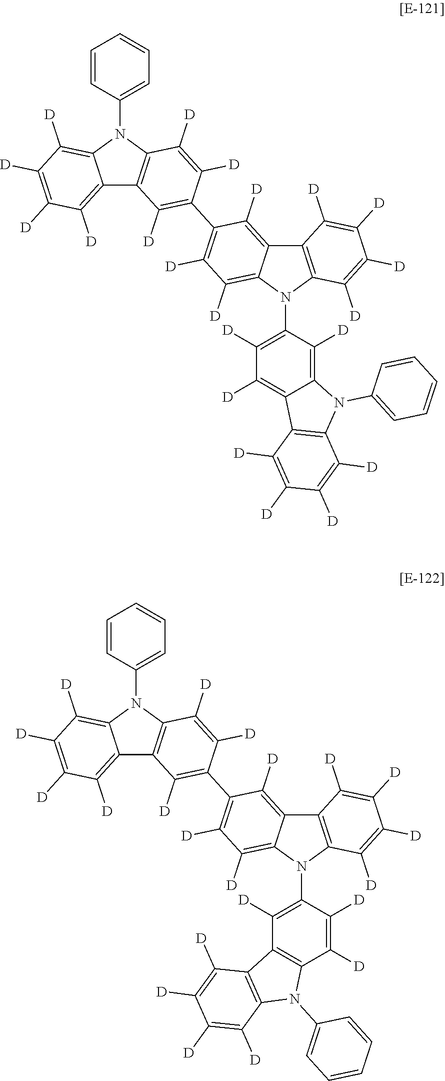 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

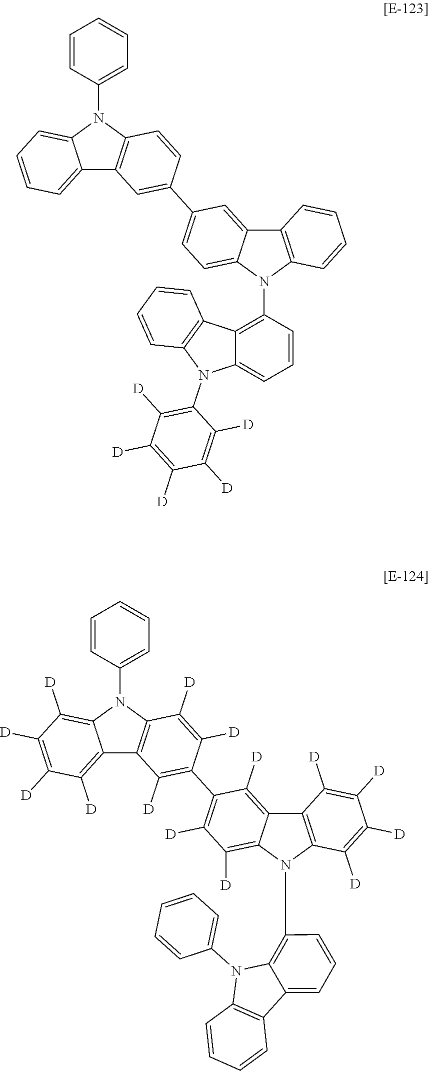 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

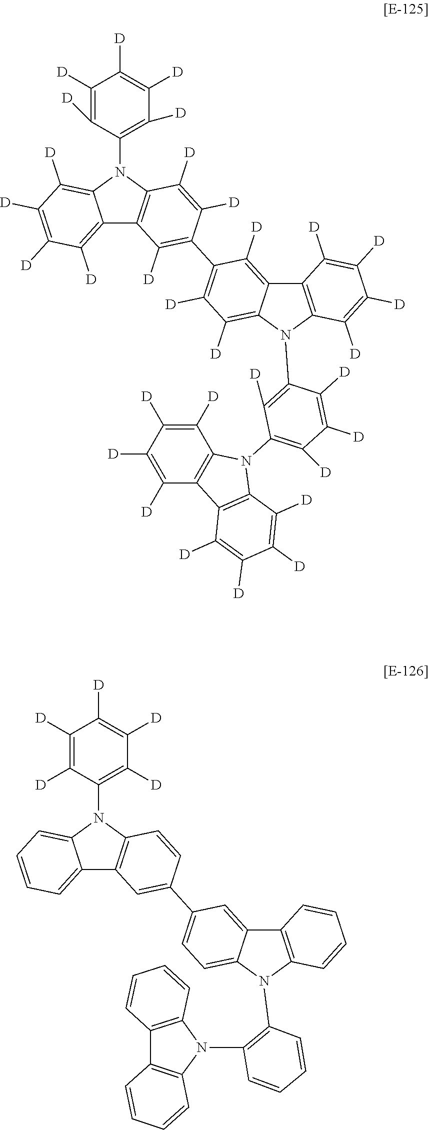 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H] ; [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

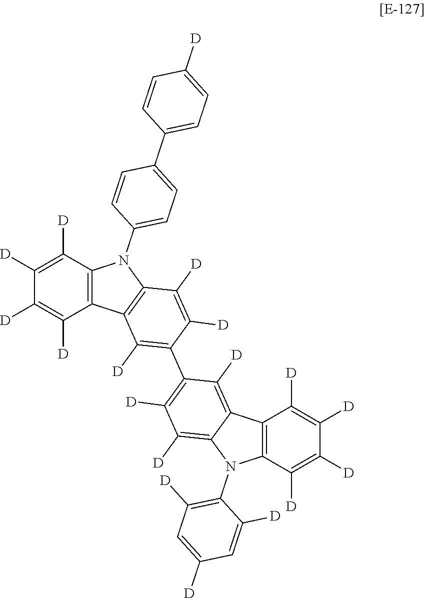 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

(Dn is the number of hydrogen atoms replaced by deuterium and indicates a structure in which one or more deuterium atoms have been replaced)

In an implementation, the second compound may be represented by a combination of Chemical Formulas 4 and 5.

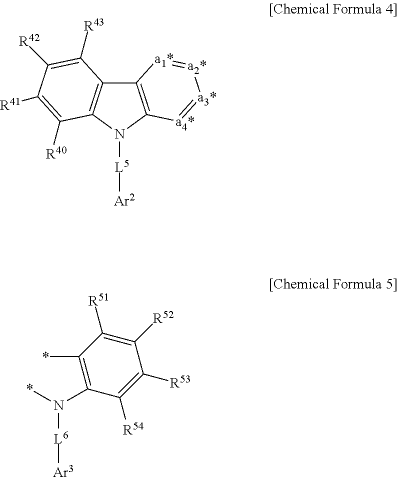

In Chemical Formulas 4 and 5, two adjacent ones of a₁* to a₄* of Chemical Formula 4 may each be linking carbons linked at * of Chemical Formula 5, the remaining two of a₁* to a₄* of Chemical Formula 4, not linked at * of Chemical Formula 5, may each independently be C-L^{b}-R^{b}.

L⁵, L⁶, and L^{b} may each independently be or include, e.g., a single bond, a substituted or unsubstituted C6 to C30 arylene group, a substituted or unsubstituted divalent C3 to C30 heterocyclic group, or a combination thereof.

Ar² and Ar³ may each independently be or include, e.g., a substituted or unsubstituted C6 to C30 aryl group or a substituted or unsubstituted C2 to C30 heterocyclic group.

R⁴⁰ to R⁴³, R⁵¹ to R⁵⁴, and R^{b} may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

R⁴⁰ to R⁴³, R⁵¹ to R⁵⁴, and R^{b} may each be separately present or two adjacent ones may be linked to each other to form a ring.

In an implementation, at least one of the substituents of Chemical Formulas 4 and 5 may be substituted with deuterium. The number of substituted deuterium atoms may be 1 to the maximum number of hydrogens in the chemical formula, e.g., from 1 to 40 or from 1 to 30.

In an implementation, the second compound represented by a combination of Chemical Formulas 4 and 5 may be represented by one of Chemical Formulas 4A to 4E.

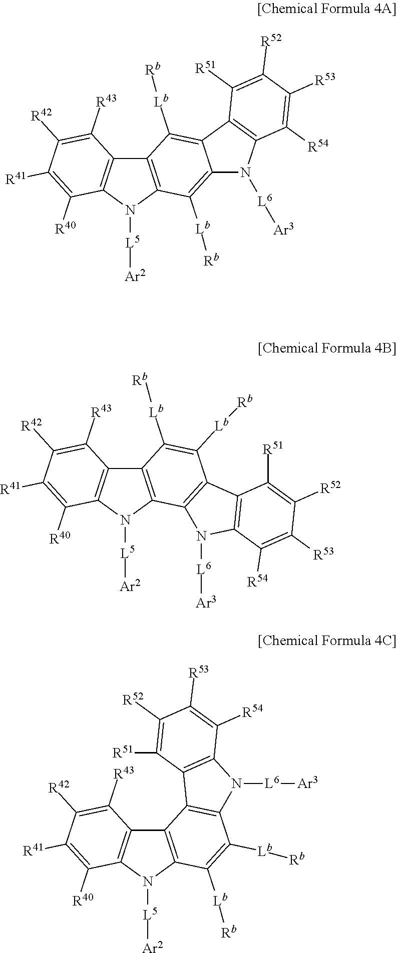

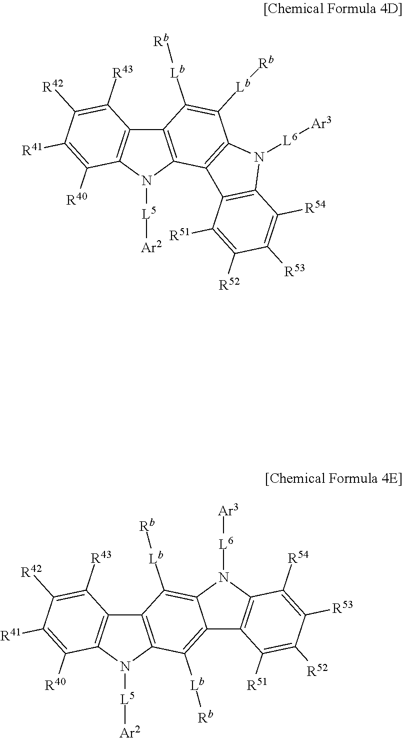

In Chemical Formulas 4A to 4E, L⁵, L⁶, L^{b}, Ar², Ar³, R⁴⁰ to R⁴³, R⁵¹ to R⁵⁴, and R^{b} may be the same as those described above.

In an implementation, in Chemical Formulas 4, 5 and 4A to 4E, L⁵, L⁶, and L^{b} may each independently be, e.g., a single bond or a substituted or unsubstituted C6 to C30 arylene group, and may be, e.g., a single bond, a substituted or unsubstituted phenylene group, a substituted or unsubstituted biphenylene group, or a substituted or unsubstituted terphenylene group.

In an implementation, in Chemical Formulas 4 and 4A to 4E, Ar² and Ar³ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

In an implementation, the second compound represented by the combination of Chemical Formulas 4 and 5 may include, e.g., a compound of Group 4.

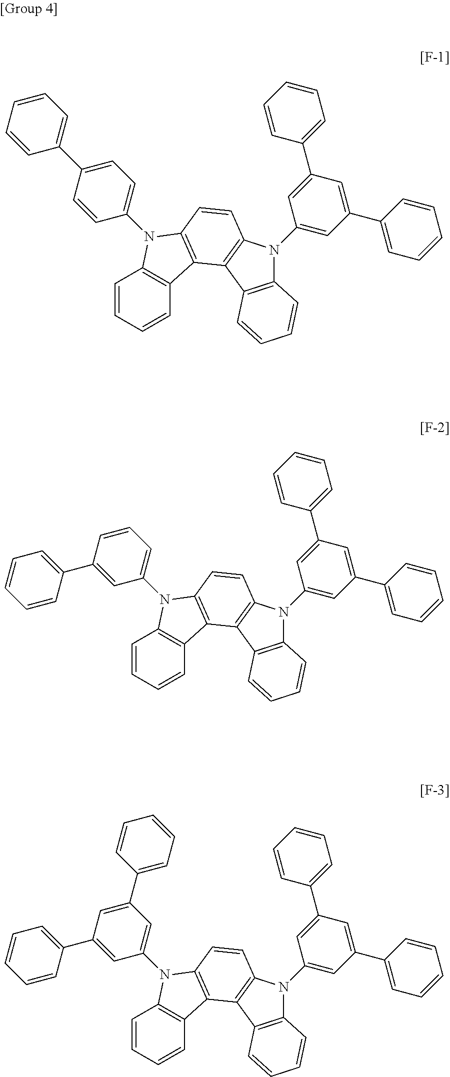 [SMILES: c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

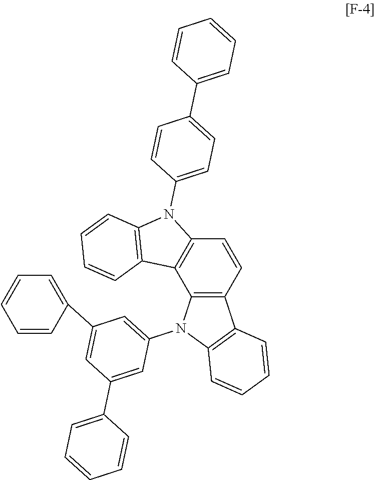 [SMILES: c1ccc(-c2ccc(-n3c4ccccc4c4c3ccc3c5ccccc5n(-c5cc(-c6ccccc6)cc(-c6ccccc6)c5)c34)cc2)cc1]

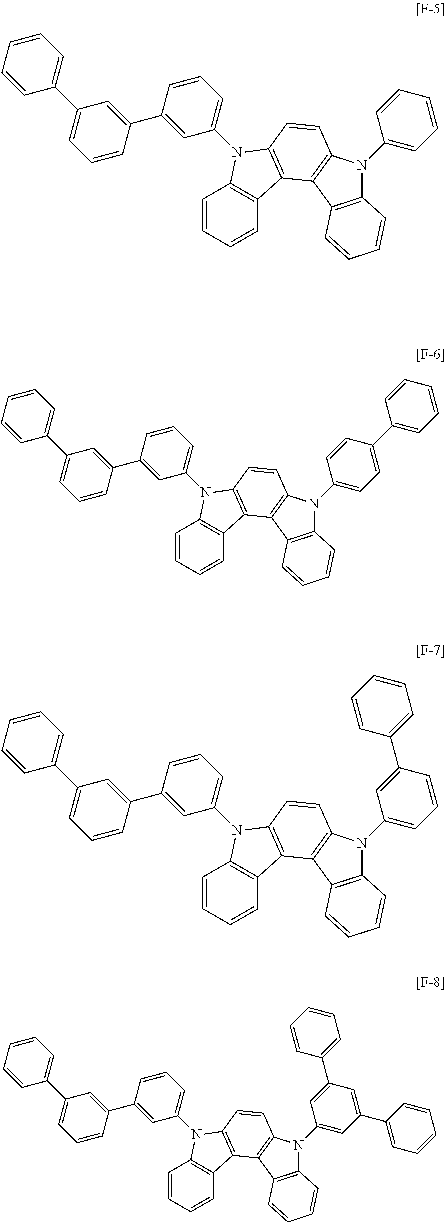 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

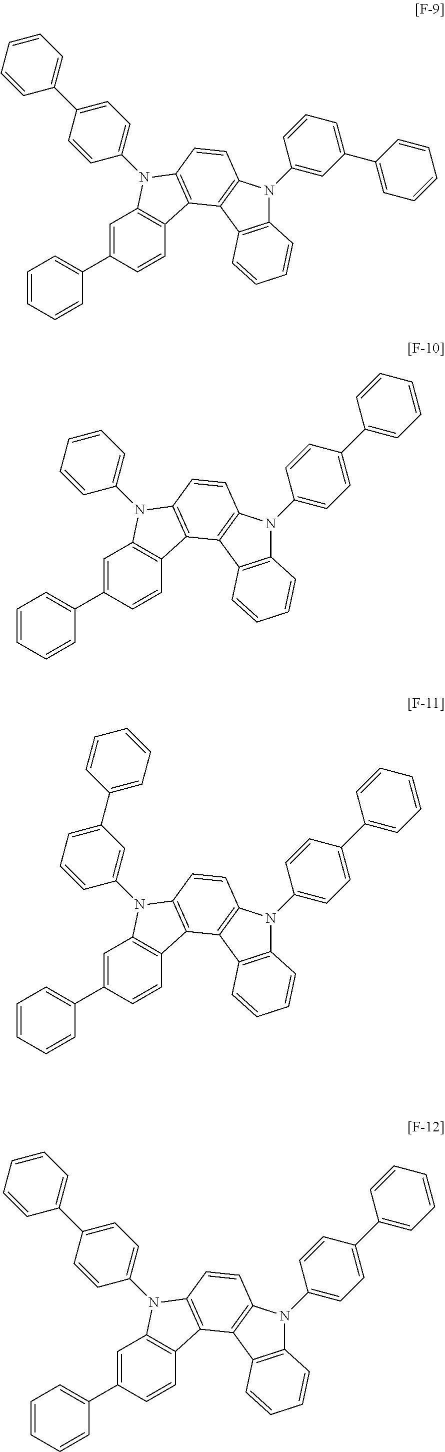 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

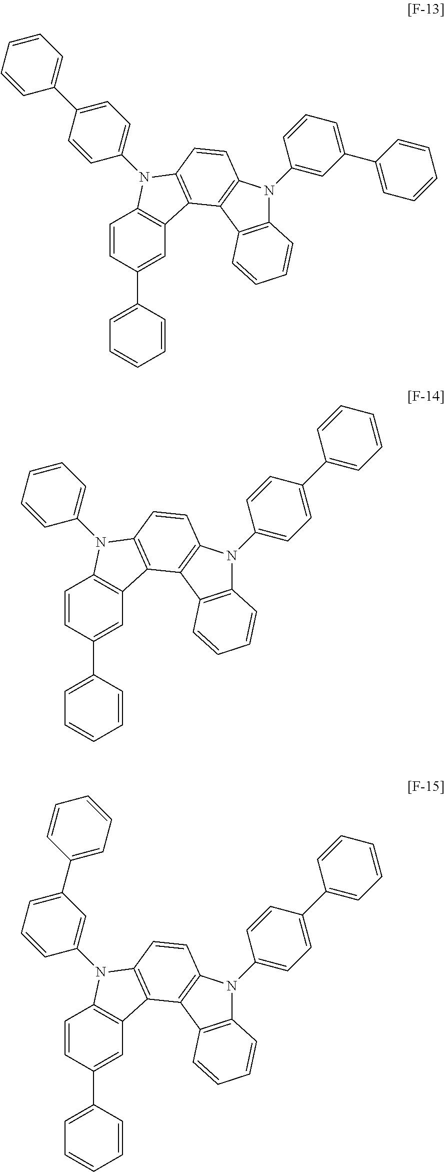 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1]

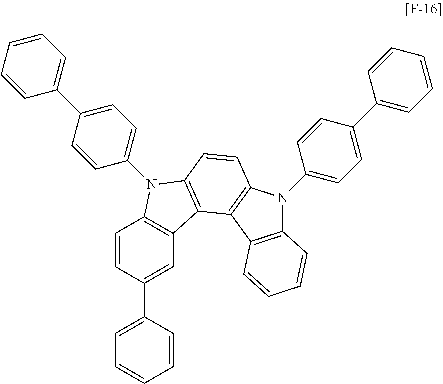 [SMILES: c1ccc(-c2ccc(-n3c4ccccc4c4c5c6cc(-c7ccccc7)ccc6n(-c6ccc(-c7ccccc7)cc6)c5ccc43)cc2)cc1]

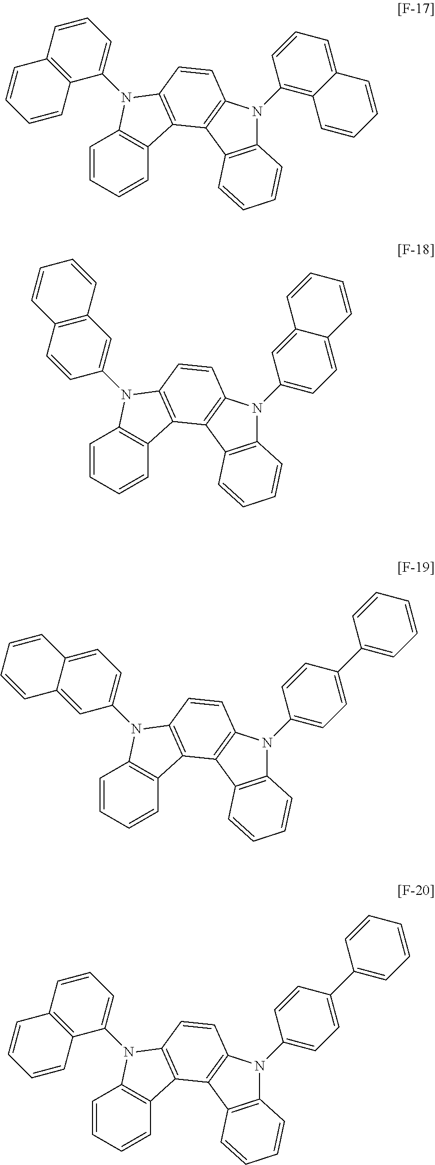 [SMILES: c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

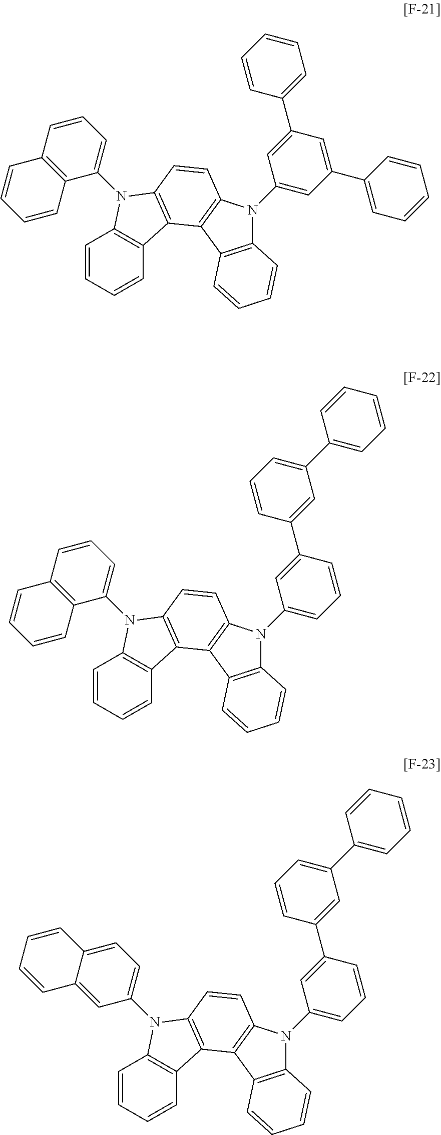 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1]

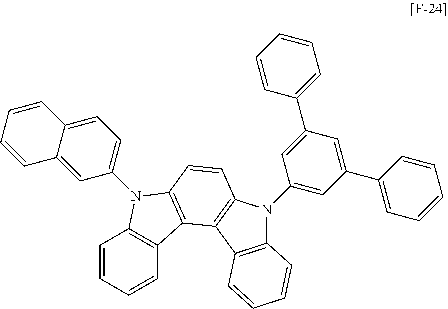 [SMILES: c1ccc(-c2cc(-c3ccccc3)cc(-n3c4ccccc4c4c5c6ccccc6n(-c6ccc7ccccc7c6)c5ccc43)c2)cc1]

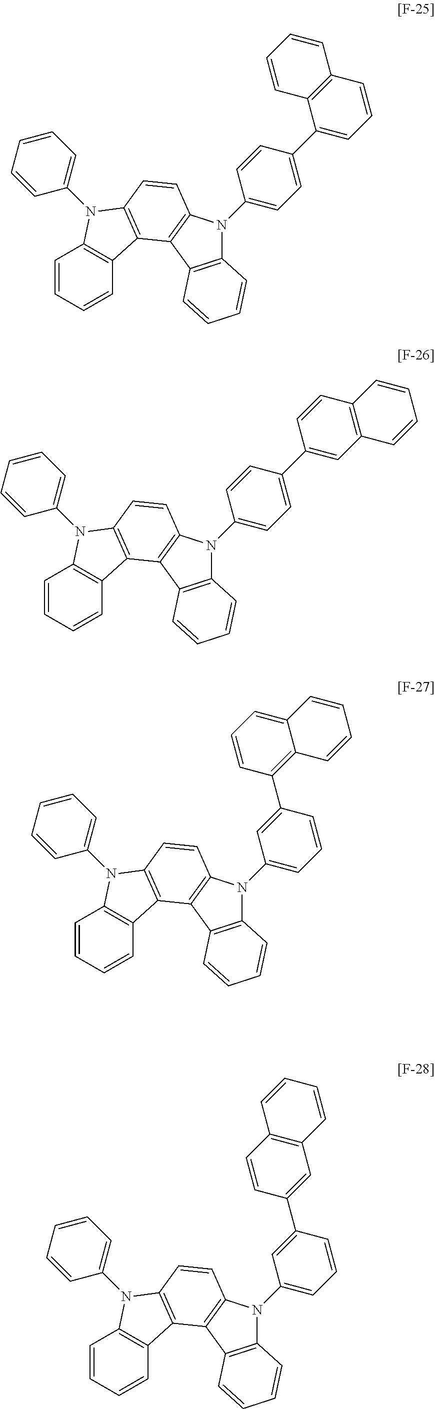 [SMILES: c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1]

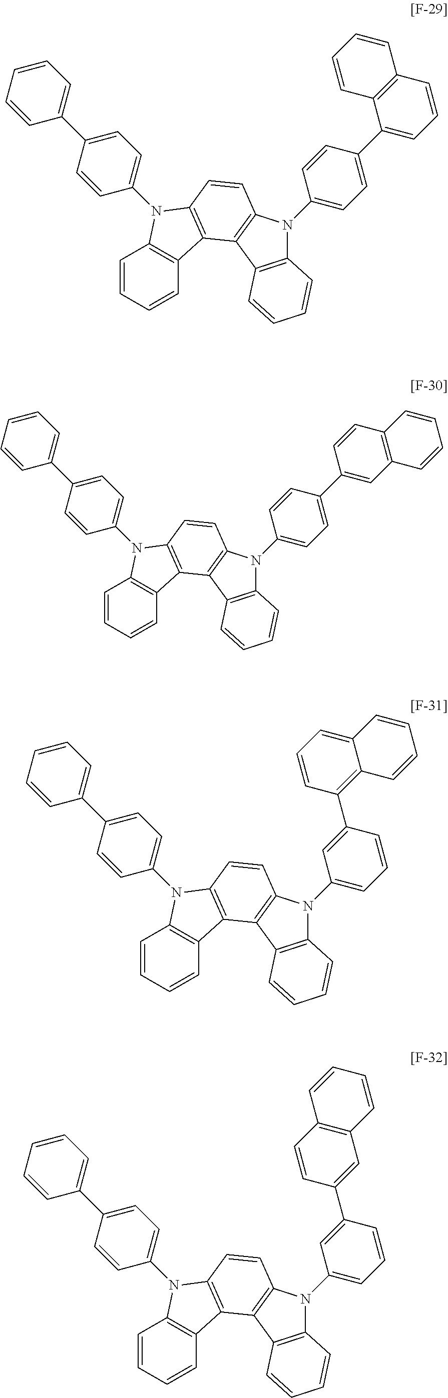 [SMILES: c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

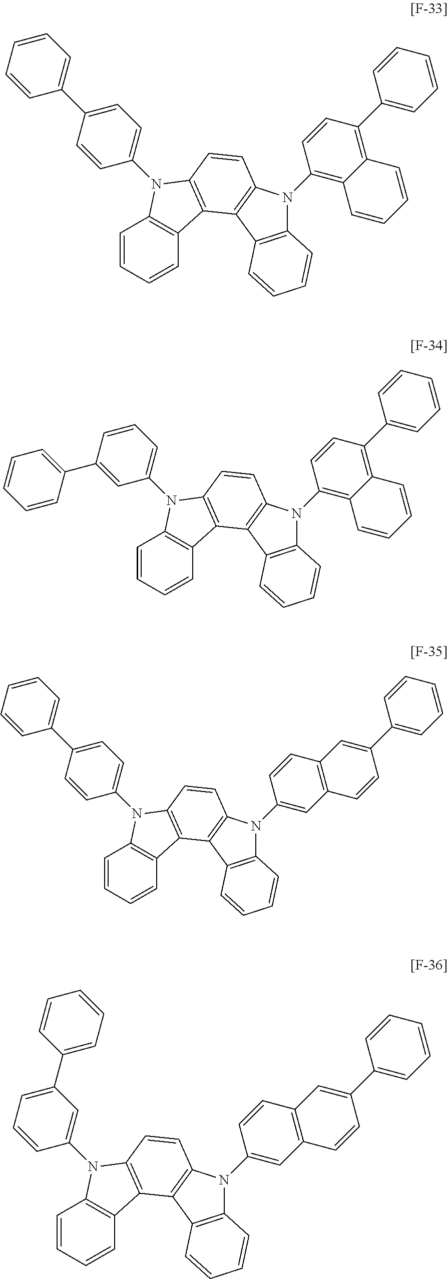 [SMILES: c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1]

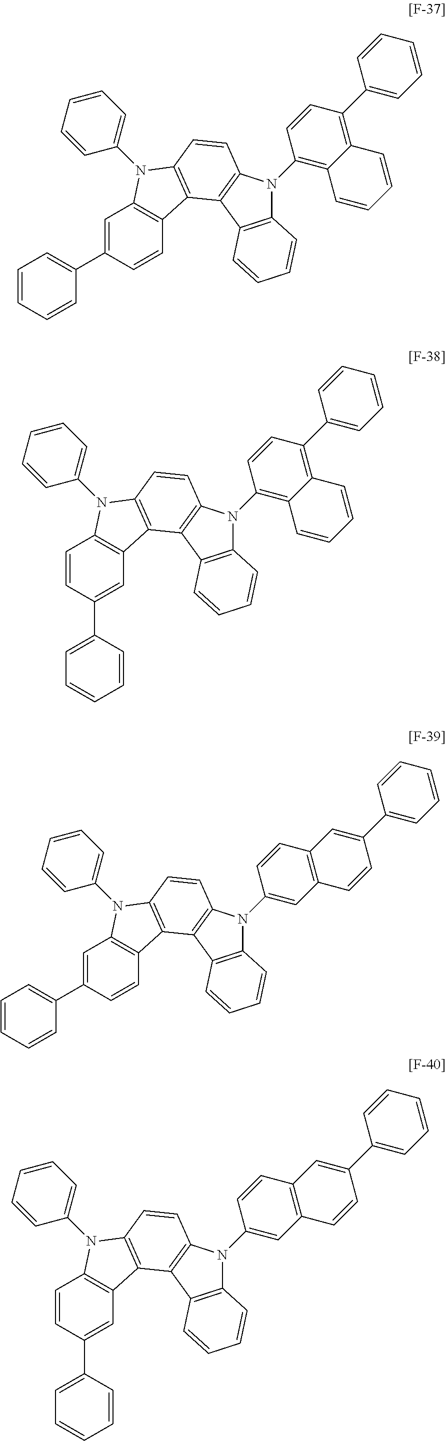 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

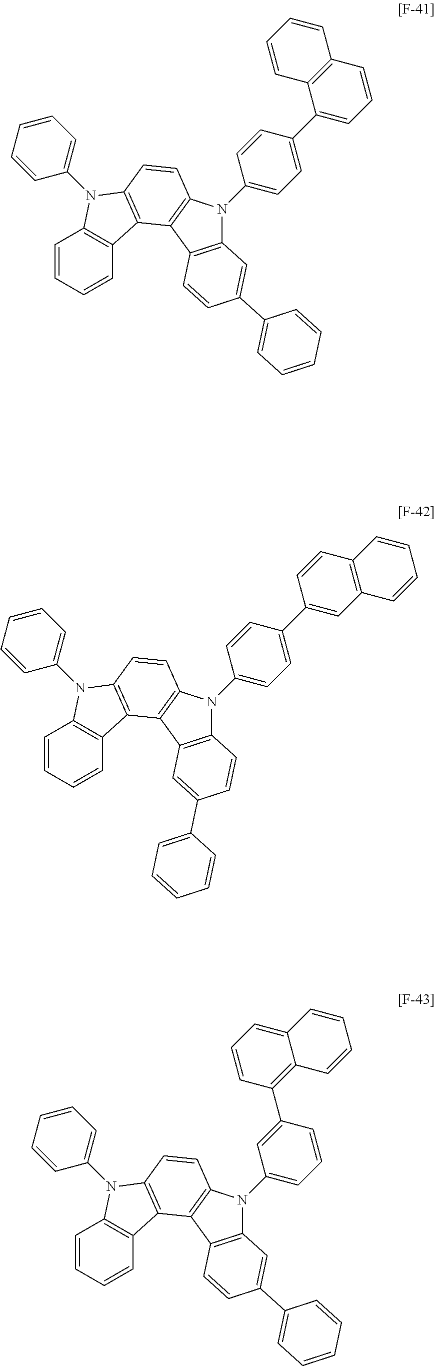 [SMILES: c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1]

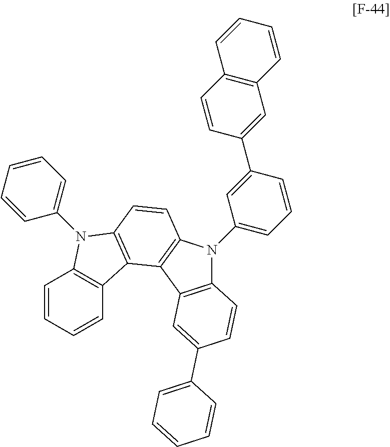 [SMILES: c1ccc(-c2ccc3c(c2)c2c4c5ccccc5n(-c5ccccc5)c4ccc2n3-c2cccc(-c3ccc4ccccc4c3)c2)cc1]

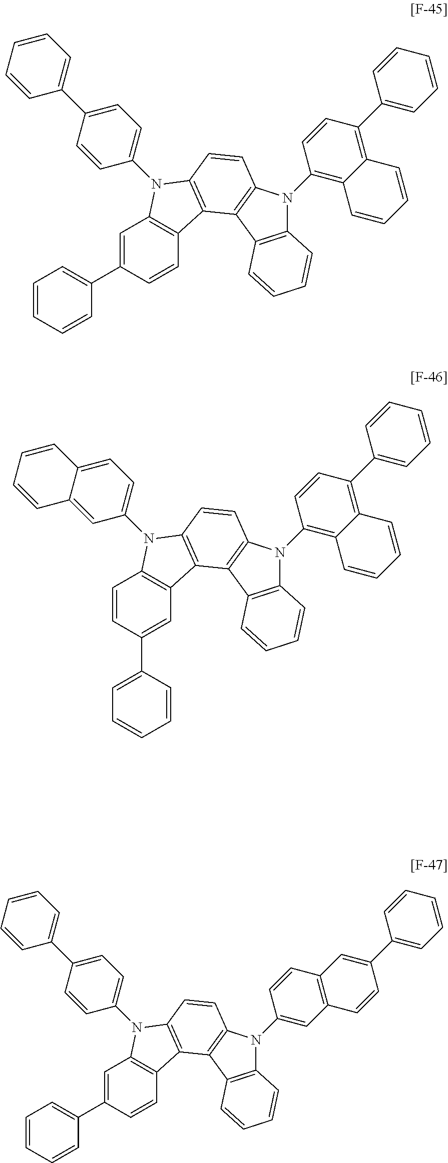 [SMILES: c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1]

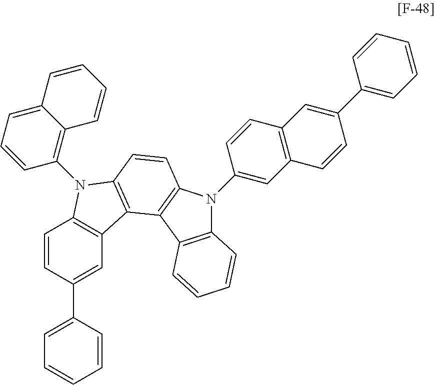 [SMILES: c1ccc(-c2ccc3cc(-n4c5ccccc5c5c6c7cc(-c8ccccc8)ccc7n(-c7cccc8ccccc78)c6ccc54)ccc3c2)cc1]

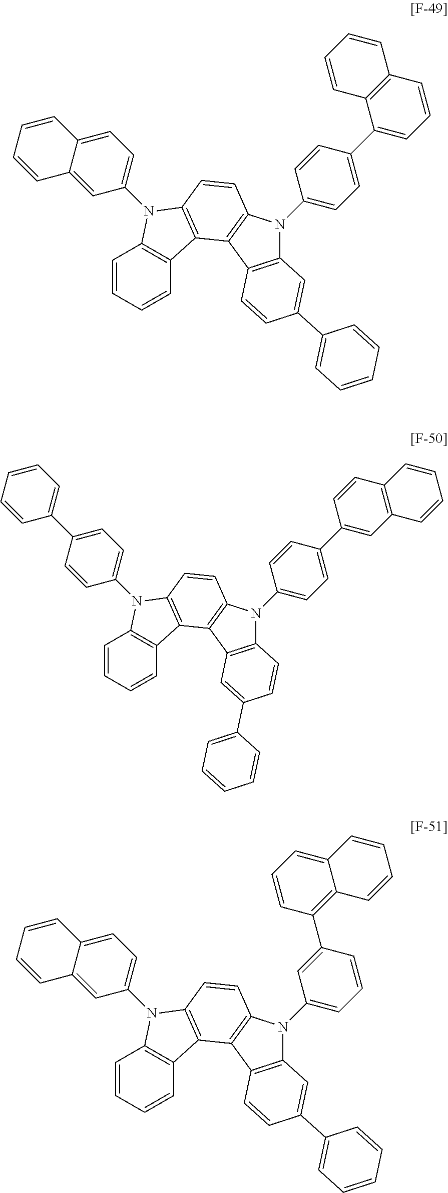 [SMILES: c1ccc(-c2ccc3c4c5c6ccccc6n(-c6ccc7ccccc7c6)c5ccc4n(-c4ccc(-c5cccc6ccccc56)cc4)c3c2)cc1 ; c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1]

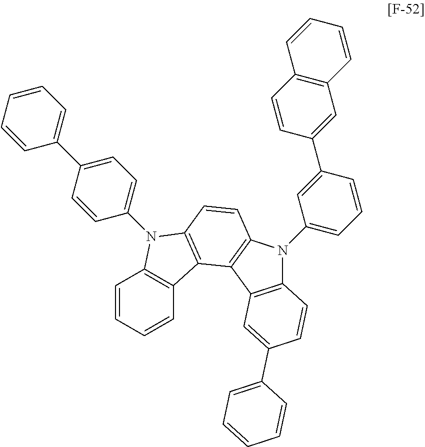 [SMILES: c1ccc(-c2ccc(-n3c4ccccc4c4c5c6cc(-c7ccccc7)ccc6n(-c6cccc(-c7ccc8ccccc8c7)c6)c5ccc43)cc2)cc1]

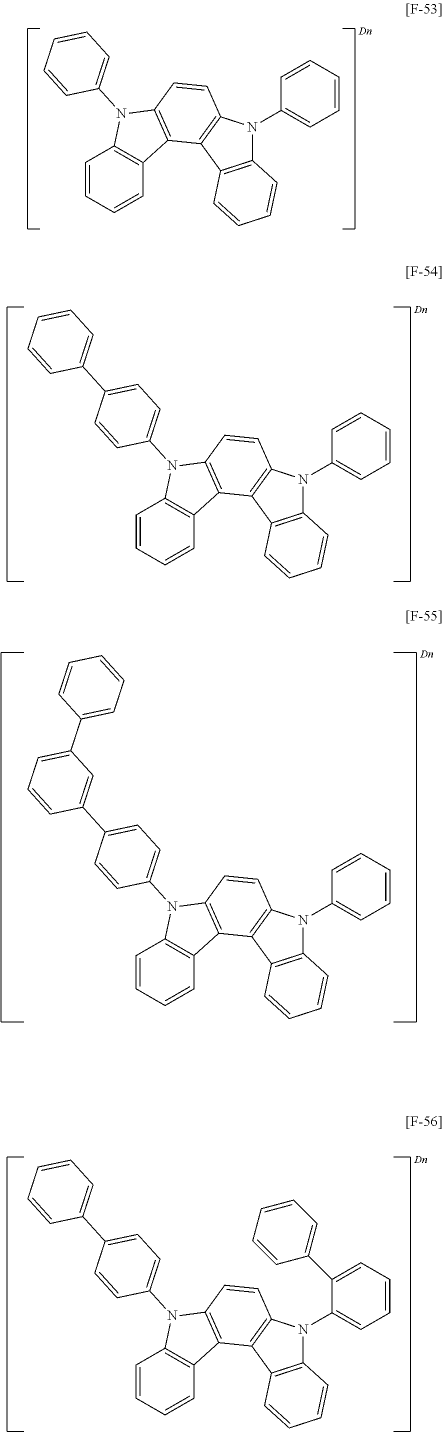

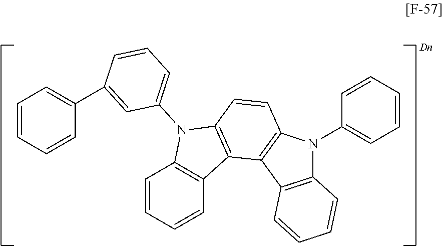

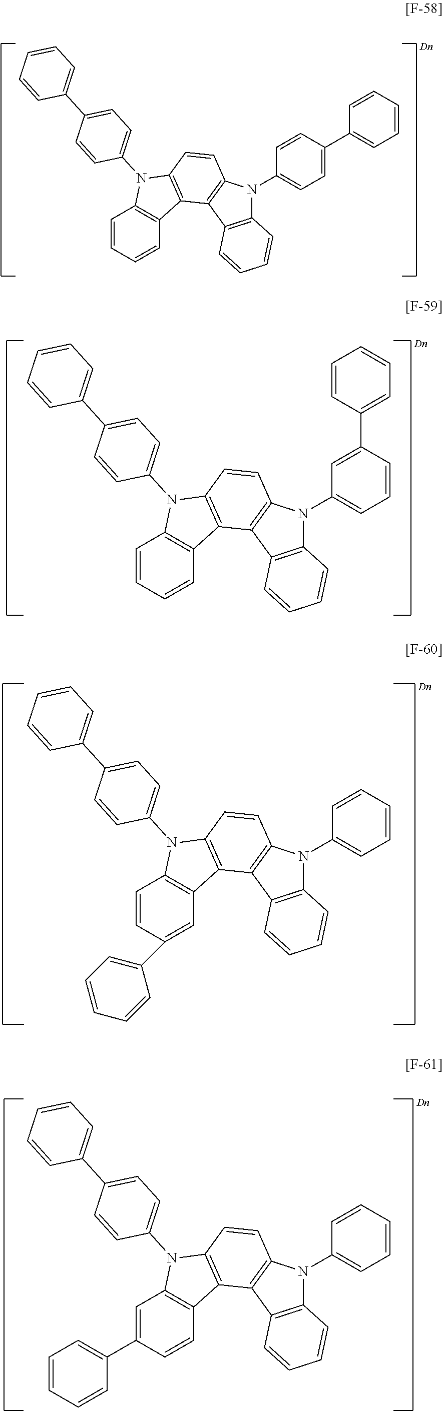

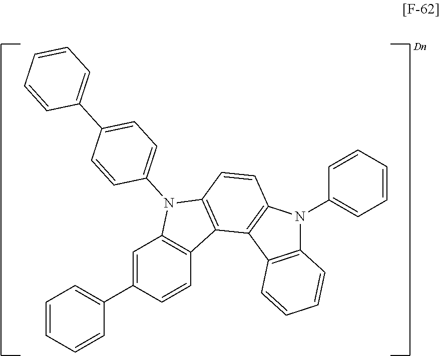

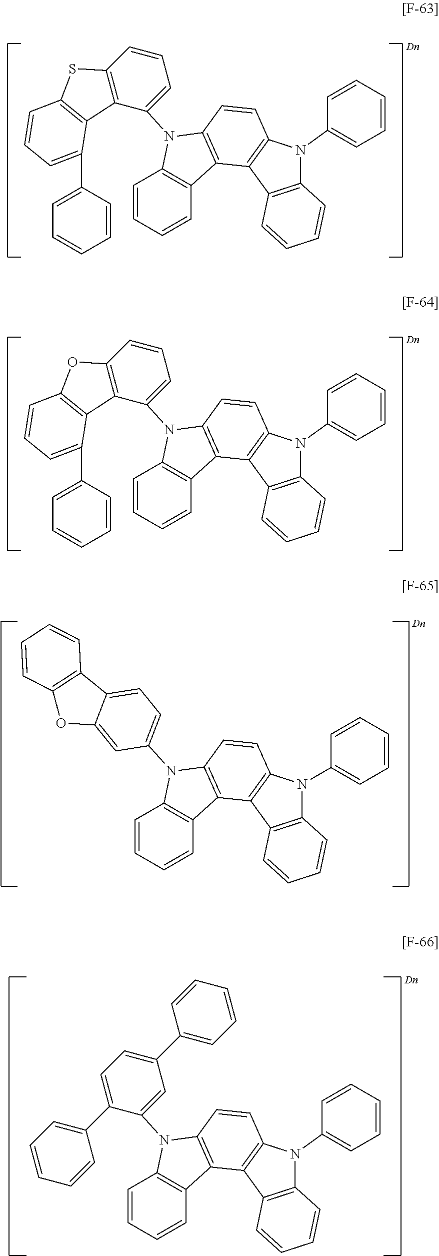

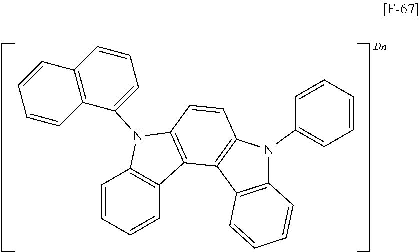

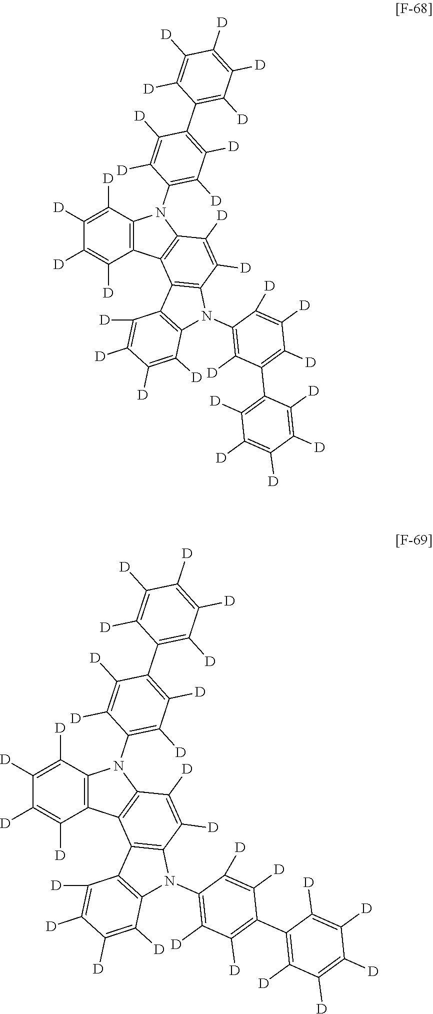 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

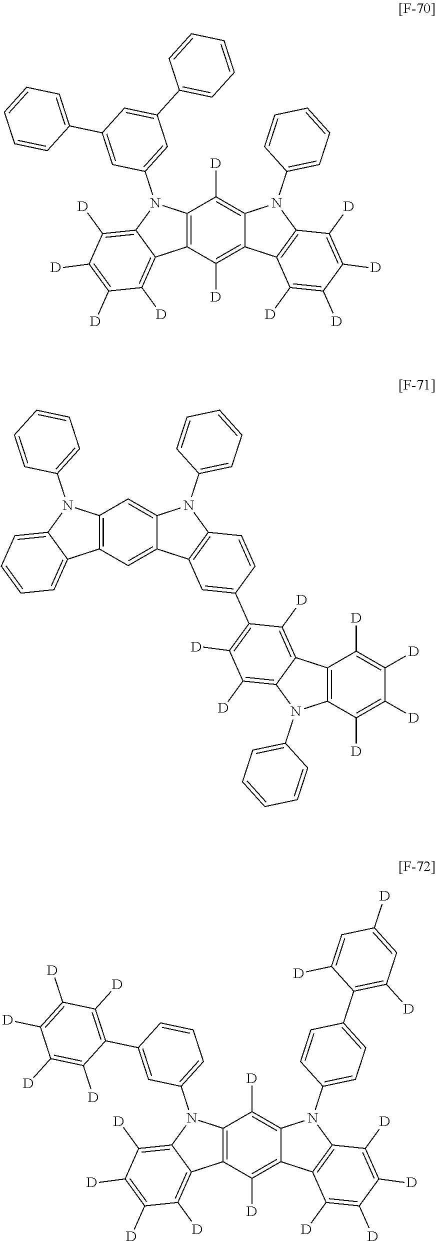 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

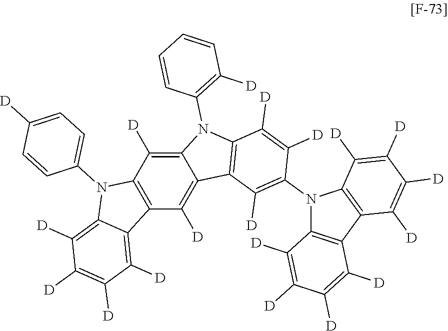 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1]

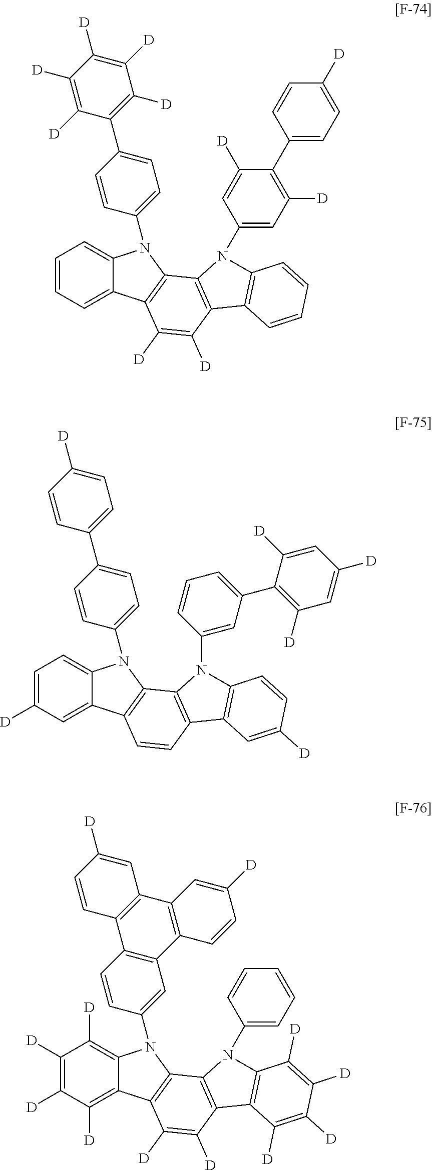 [SMILES: [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1 ; [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1]

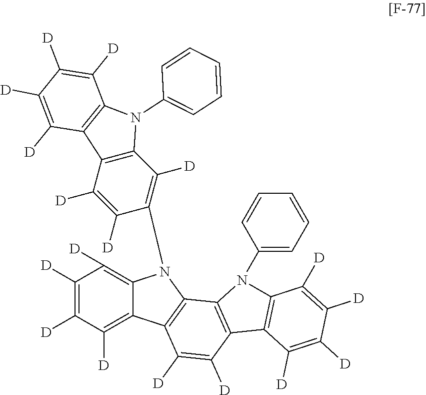 [SMILES: [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1]

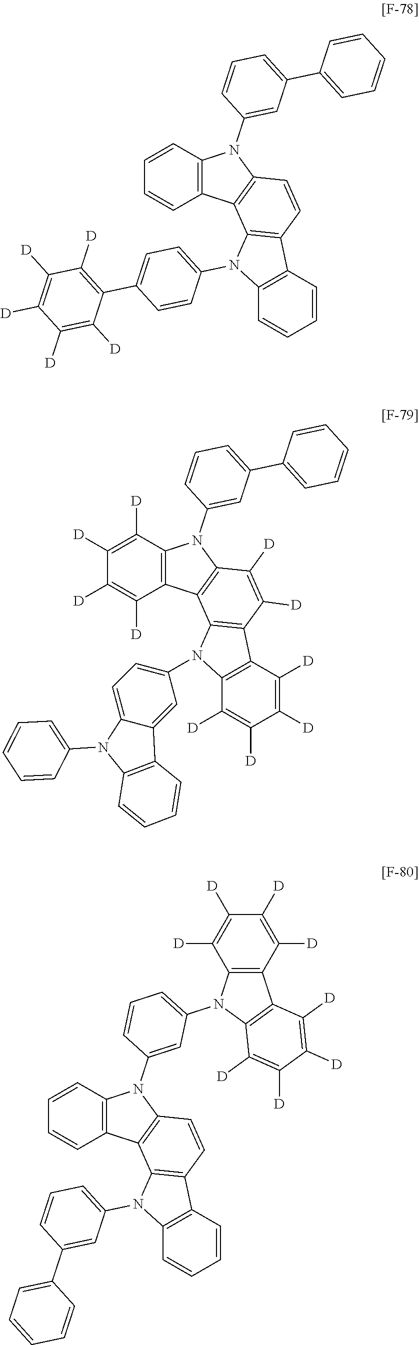 [SMILES: [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1 ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

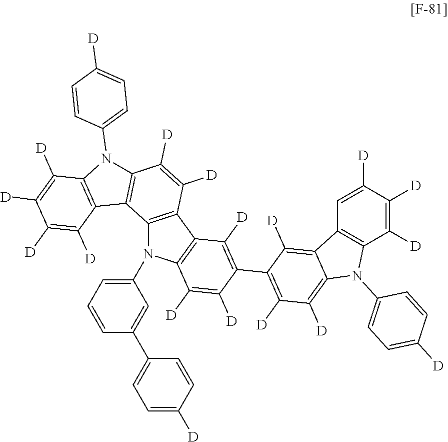 [SMILES: [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1]

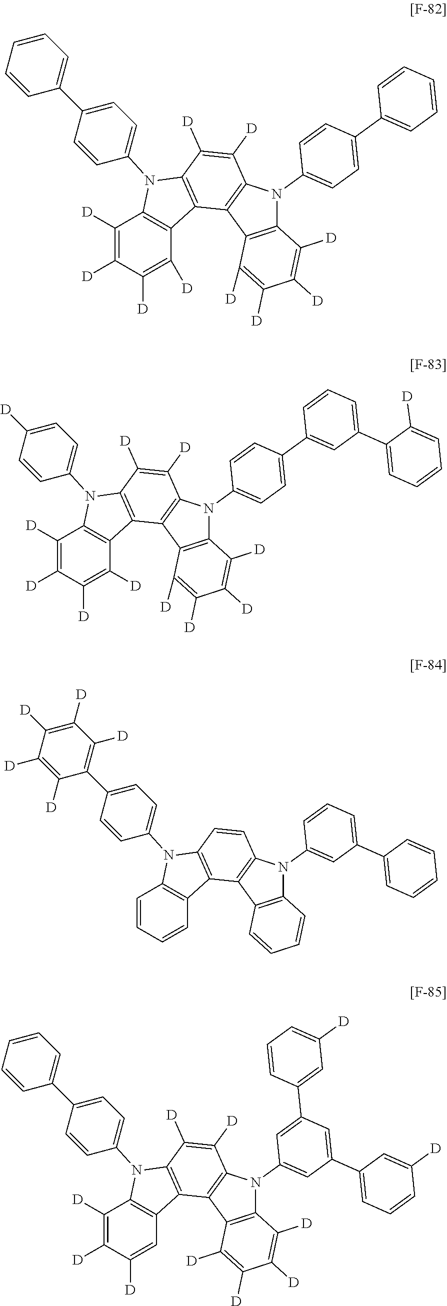

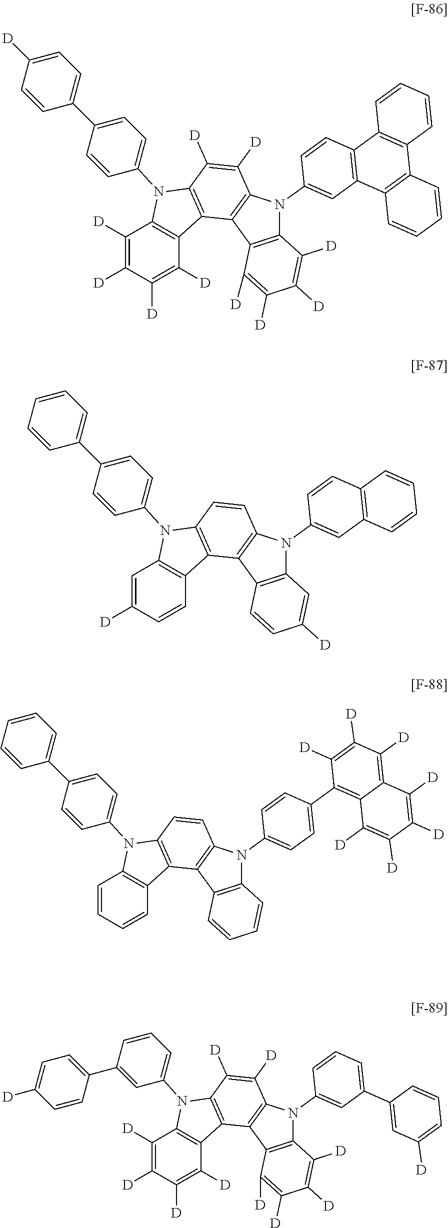 [SMILES: [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1 ; [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H] ; [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1]

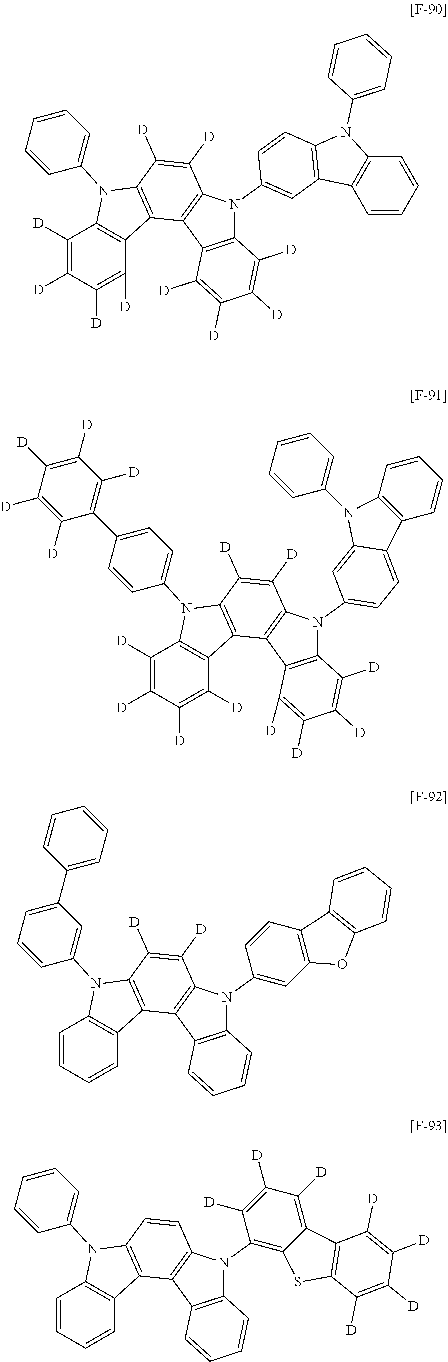 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1 ; [2H]c1c([2H])c([2H])c(-c2ccc(-n3c4c([2H])c([2H])c([2H])c([2H])c4c4c5c6c([2H])c([2H])c([2H])c([2H])c6n(-c6ccc7c8ccccc8n(-c8ccccc8)c7c6)c5c([2H])c([2H])c43)cc2)c([2H])c1[2H] ; [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

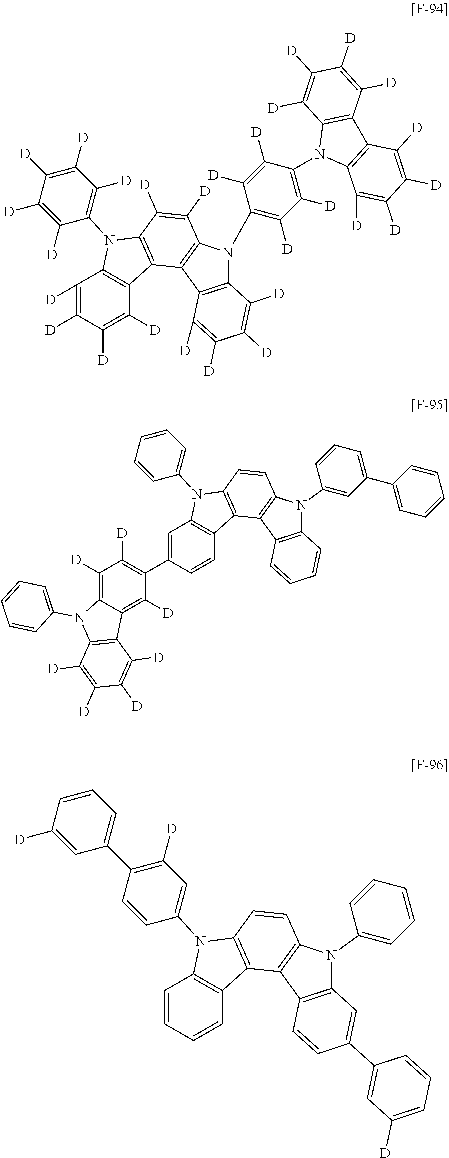 [SMILES: [2H]c1c([2H])c([2H])c(-n2c3c([2H])c([2H])c([2H])c([2H])c3c3c4c5c([2H])c([2H])c([2H])c([2H])c5n(-c5c([2H])c([2H])c(-n6c7c([2H])c([2H])c([2H])c([2H])c7c7c([2H])c([2H])c([2H])c([2H])c76)c([2H])c5[2H])c4c([2H])c([2H])c32)c([2H])c1[2H] ; [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1 ; [2H]c1cccc(-c2ccc3c4c5c6ccccc6n(-c6ccc(-c7cccc([2H])c7)c([2H])c6)c5ccc4n(-c4ccccc4)c3c2)c1]

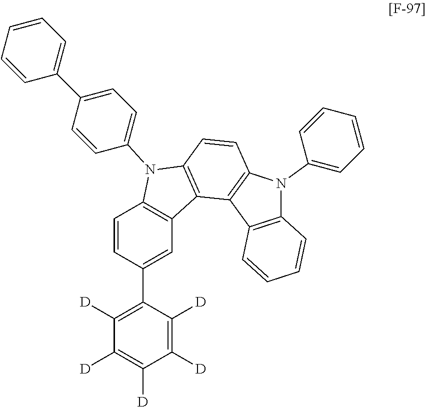 [SMILES: [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

(Dn is the number of hydrogen atoms replaced by deuterium and indicates a structure in which one or more deuterium atoms have been replaced)

The first compound and the second compound may each be a bipolar compound having both electron and hole characteristics, and the first compound may be a bipolar compound having relatively strong electron characteristics, and the second compound may be a bipolar compound having relatively strong hole characteristics. Additionally, the first compound and the second compound may exhibit good interfacial characteristics due to their structures.

The composition may finely control the mobility of holes and electrons by including the first compound and the second compound together, thereby achieving a balance of holes and electrons in an active layer (e.g., a light emitting layer) of an organic optoelectronic device.

The composition may include the first compound and the second compound in various ratios. In an implementation, the composition may include the first compound and the second compound in a weight ratio of, e.g., about 10:90 to about 90:10, and within that range, may include the first compound and the second compound in a weight ratio of, e.g., about 20:80 to about 80:20, about 30:70 to about 70:30, about 40:60 to about 60:40, or about 50:50.

In an implementation, the first compound may be included in an amount equal to or greater than the second compound. For example, the first compound may be included in an amount of about 50 wt % to about 90 wt %, based on a total weight of the first compound and the second compound.

In an implementation, the first compound may be included in less or the same amount as the second compound. For example, the first compound may be included in an amount of about 10 wt % to about 50 wt %, based on a total weight of the first compound and the second compound.

The composition may further include a light emitting dopant in addition to the first compound and the second compound. The light emitting dopant may be a substance that is mixed in a small amount in the composition and may cause luminescence, and may be, e.g., a fluorescent dopant, a phosphorescent dopant, or a combination thereof. The light emitting dopant may generally be a material, e.g., a metal complex, configured to emit light by multiple excitation into a triplet or more. The light emitting dopant may be, e.g., an inorganic, organic, or organic/inorganic compound, and may be included in one or more types.

The fluorescent dopant may be a fused polycyclic compound containing, e.g., boron (B), nitrogen (N), or a combination thereof, and may be, e.g., one of Compounds D1 to D30.

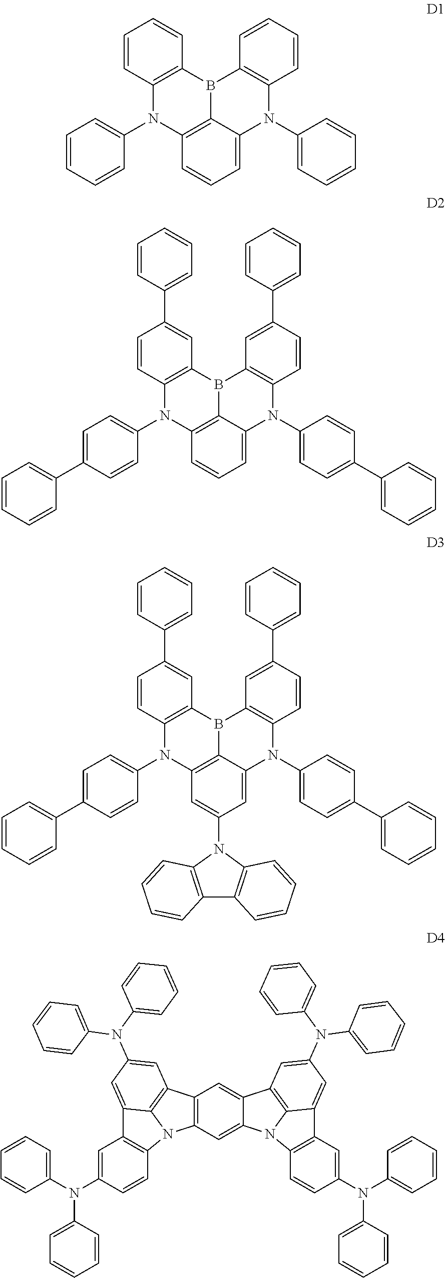 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1 ; c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1]

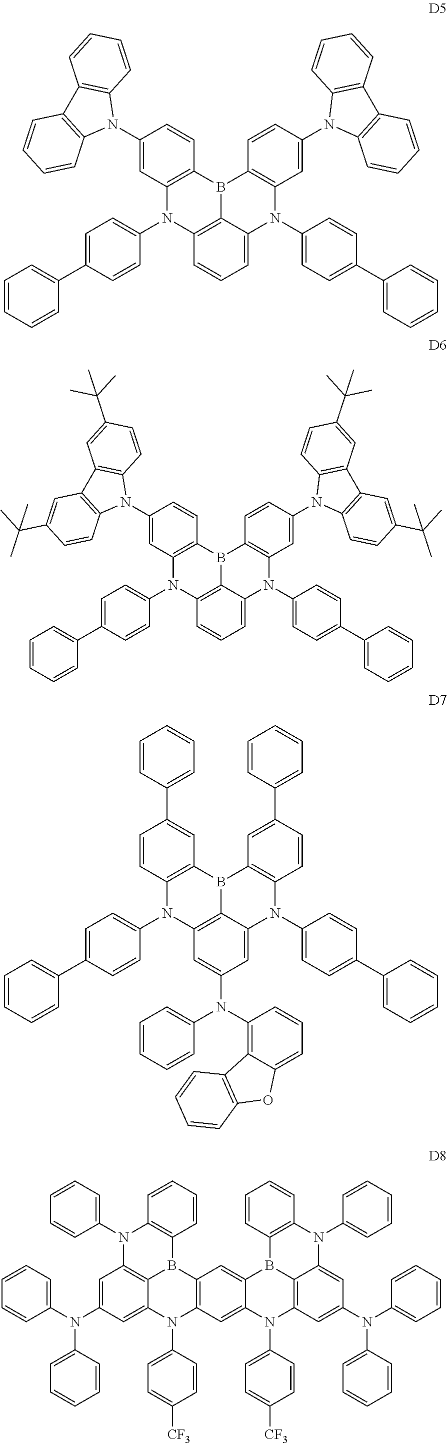

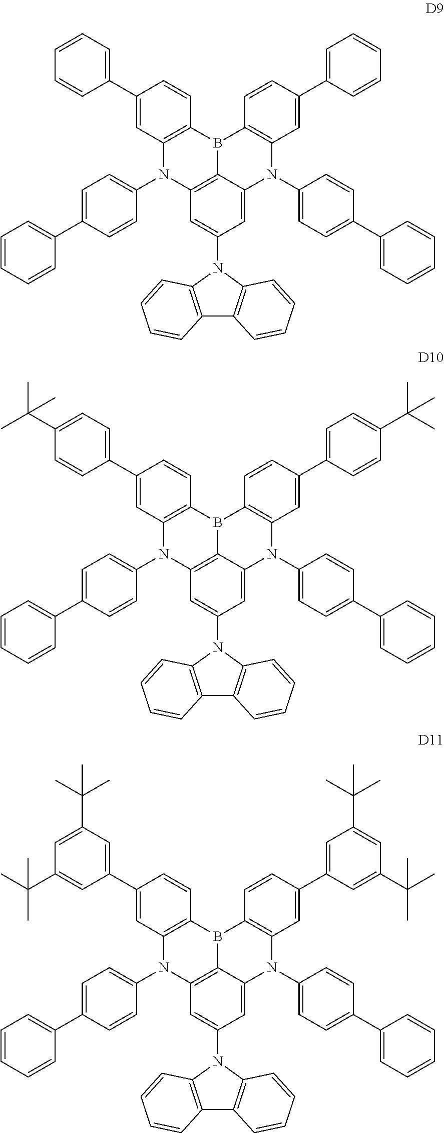 [SMILES: c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1 ; CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1]

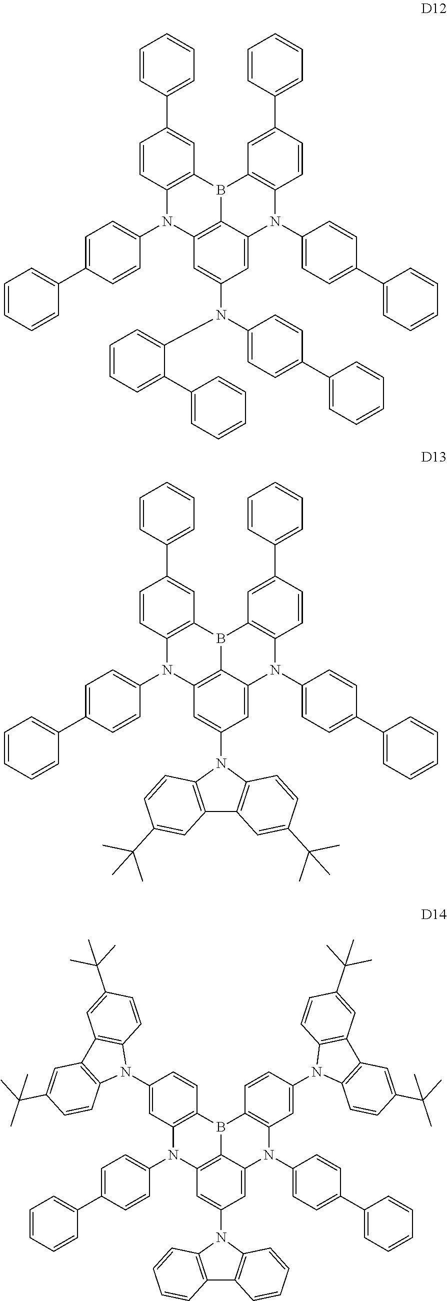 [SMILES: c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1 ; CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1]

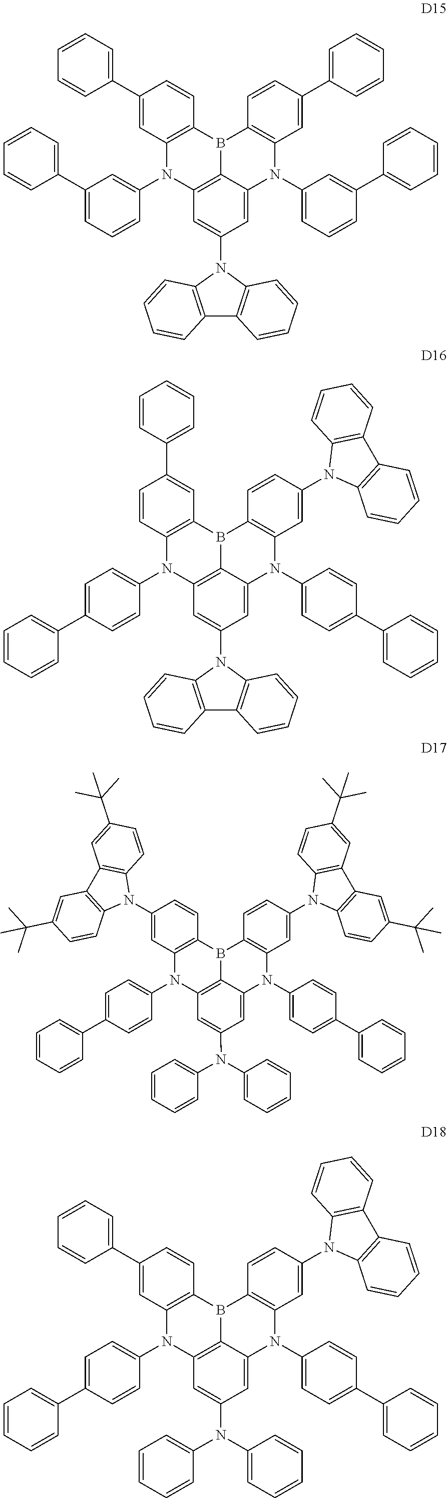

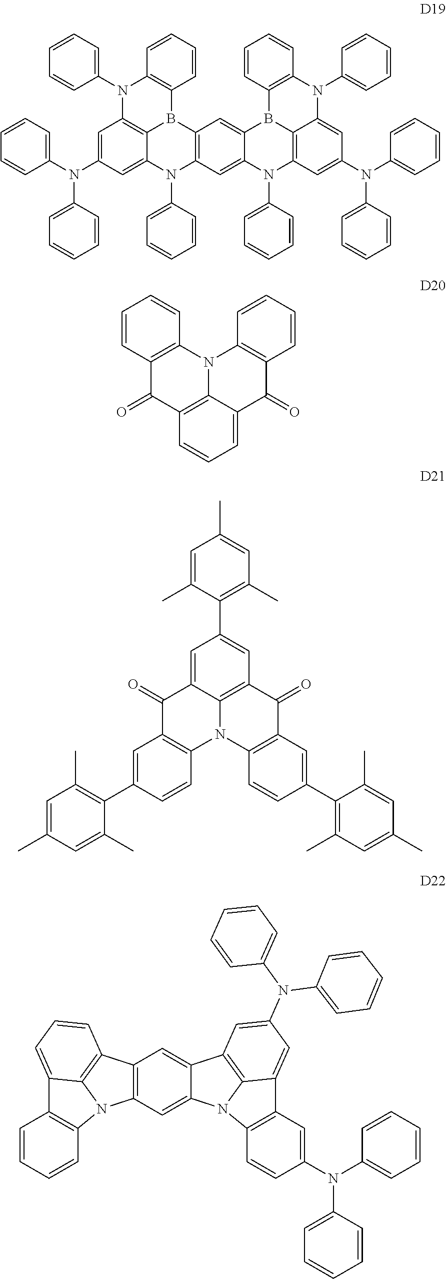

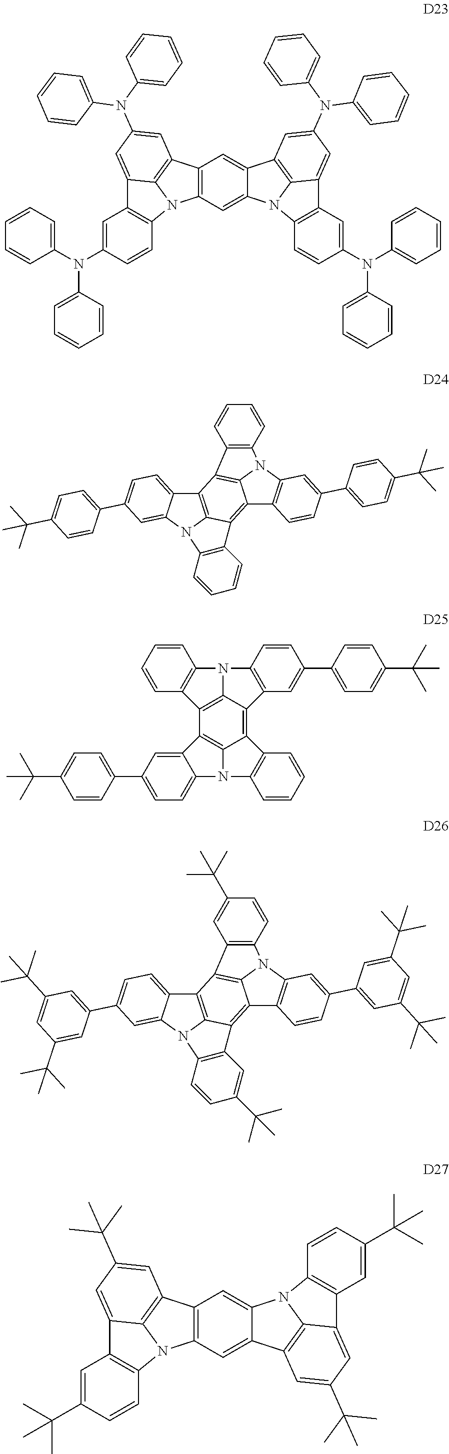

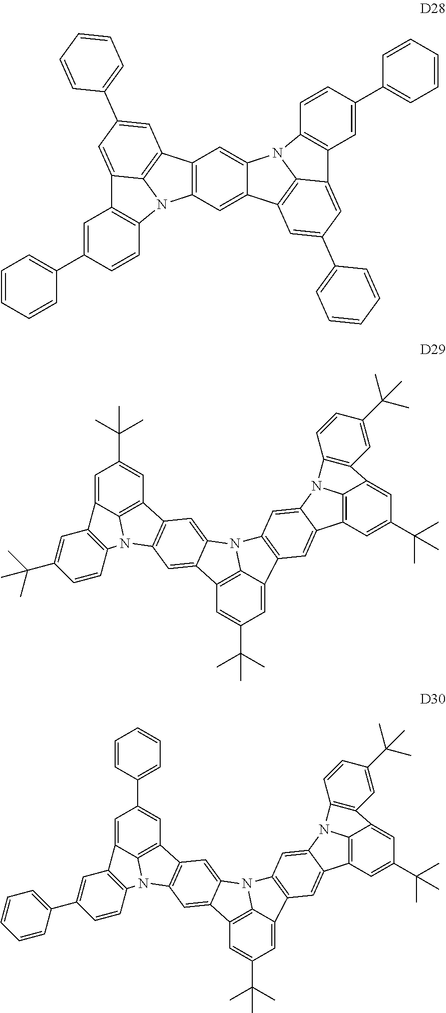 [SMILES: c1ccc(-c2ccc3c(c2)c2cc(-c4ccccc4)cc4c5cc6c(cc5n3c24)c2cc(-c3ccccc3)cc3c4cc(-c5ccccc5)ccc4n6c32)cc1 ; CC(C)(C)c1ccc2c(c1)c1cc(C(C)(C)C)cc3c4cc5c(cc4n2c13)c1cc(C(C)(C)C)cc2c3cc4c6cc(C(C)(C)C)cc7c8cc(C(C)(C)C)ccc8n(c4cc3n5c12)c76 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-c2ccccc2)cc2c3cc(-c4ccccc4)ccc3n9c21]

The phosphorescent dopant may include organometallic compounds including, e.g., Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

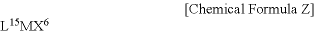

In Chemical Formula Z, M may be a metal, e.g., Ir, Pt, Os, Ti, Zr, Hf, Eu, Tb, Tm, Fe, Co, Ni, Ru, Rh, Pd, or a combination thereof.

L¹⁵ and X⁶ may be the same or different and may be ligands that form a complex with M, and may be, e.g., a bidentate ligand.

The ligands represented by L¹⁵ and X⁶ may be, e.g., represented by one of Chemical Formula Z-1 to Chemical Formula Z-8.

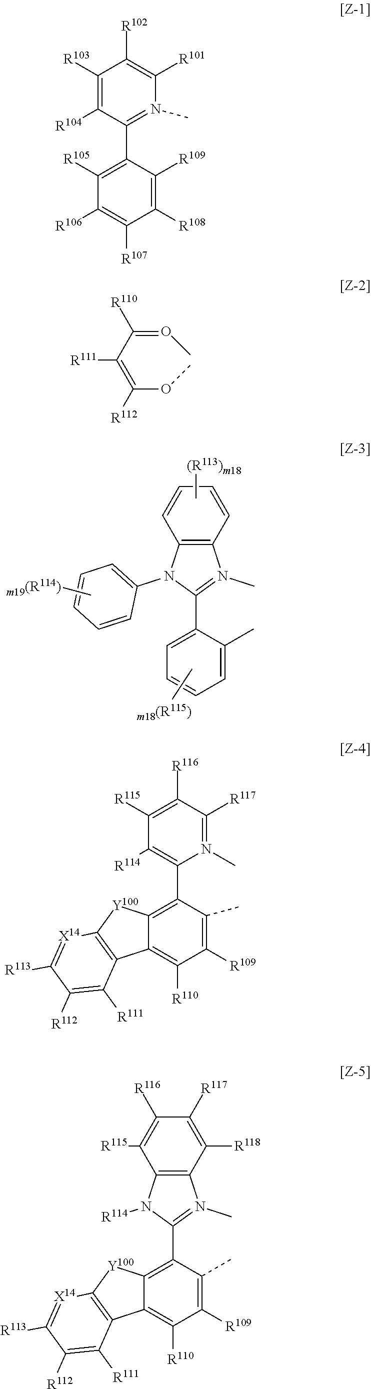

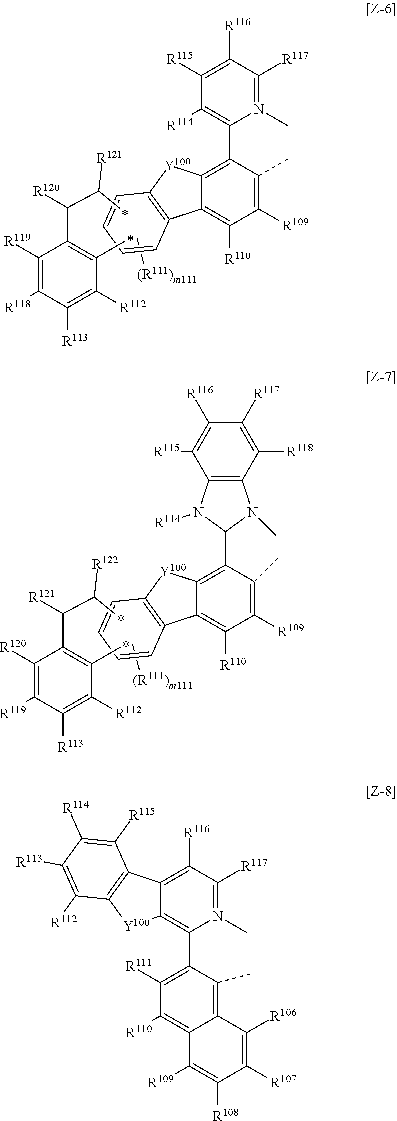

In Chemical Formula Z-1 to Chemical Formula Z-8, X¹⁴ may be, e.g., carbon or nitrogen.

Y¹⁰⁰ may be, e.g., O or S.

R¹⁰¹ to R¹²² may each independently be, e.g., hydrogen, deuterium, a halogen, a substituted or unsubstituted C1 to C30 alkyl group, a substituted or unsubstituted C3 to C30 cycloalkyl group, a substituted or unsubstituted C6 to C20 aryl group, —SiR¹³³R¹³⁴R¹³⁵, or —GeR¹³³R¹³⁴R¹³⁵ (wherein, R¹³³ to R¹³⁵ are independently hydrogen, deuterium, a substituted or unsubstituted C1 to C30 alkyl group, a substituted or unsubstituted C3 to C30 cycloalkyl group, or a substituted or unsubstituted C6 to C20 aryl group); or may be linked to adjacent substituents to form a substituted or unsubstituted ring, and, e.g., together with pyridine, may be a substituted or unsubstituted quinoline, a substituted or unsubstituted benzfuropyridine, a substituted or unsubstituted benzothienopyridine, a substituted or unsubstituted indenopyridine, a substituted or unsubstituted benzfuroquinoline, a substituted or unsubstituted benzothienoquinoline, or a substituted or unsubstituted indenoquinoline.

m18 may be, e.g., an integer of 1 to 4.

m19 may be, e.g., an integer of 1 to 5.

m111 may be, e.g., an integer of 1 or 2.

The light emitting dopant may further include a phosphorescent sensitizer. The phosphorescent sensitizer may be an organometallic compound and may effectively transfer energy received from the host to the fluorescent dopant. The phosphorescent sensitizer may increase energy transfer to the fluorescent dopant, causing excitons formed in the light emitting layer to emit light quickly inside the light emitting layer, thereby reducing deterioration of the light emitting diode.

The phosphorescent sensitizer may be, e.g., an organo-metal compound including iridium (Ir), platinum (Pt), osmium (Os), titanium (Ti), zirconium (Zr), hafnium (Hf), europium (Eu), terbium (Tb), thulium (Tm), rhodium (Rh), or a combination thereof, and may be, e.g., an organo-metal compound including an organic ligand including a nitrogen-containing ring. The nitrogen-containing ring may be, e.g., a substituted or unsubstituted pyridine, a substituted or unsubstituted pyrimidine, a substituted or unsubstituted triazine, a substituted or unsubstituted carbazole, a substituted or unsubstituted imidazole, a substituted or unsubstituted benzoimidazole, or a combination thereof.

The phosphorescent sensitizer may be, e.g., one of Compounds P1 to P52.

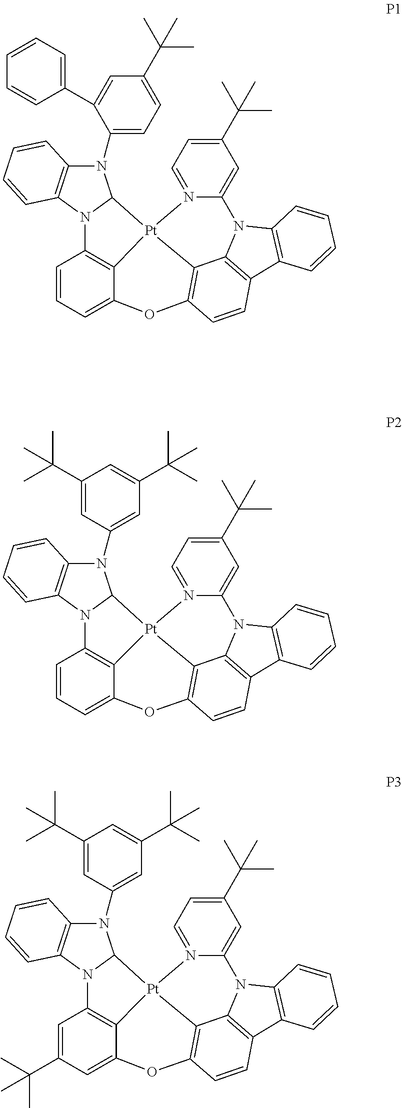

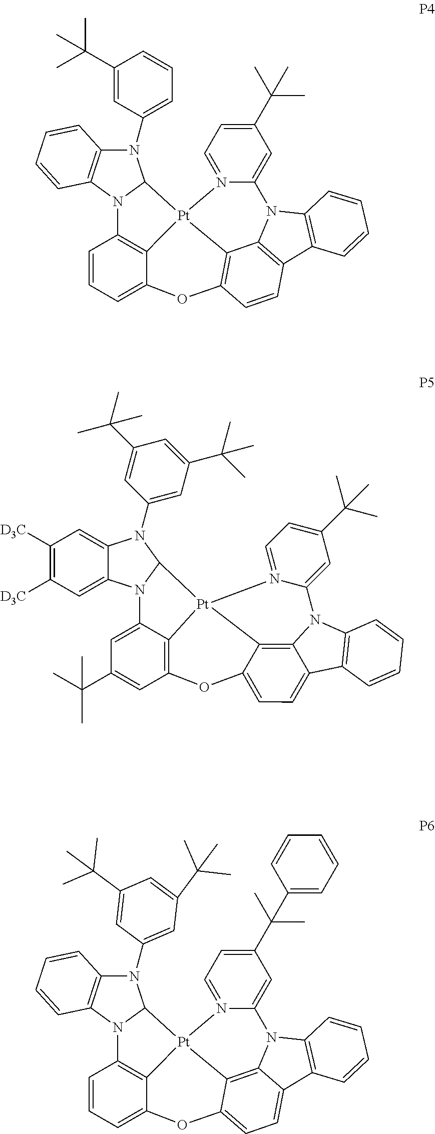

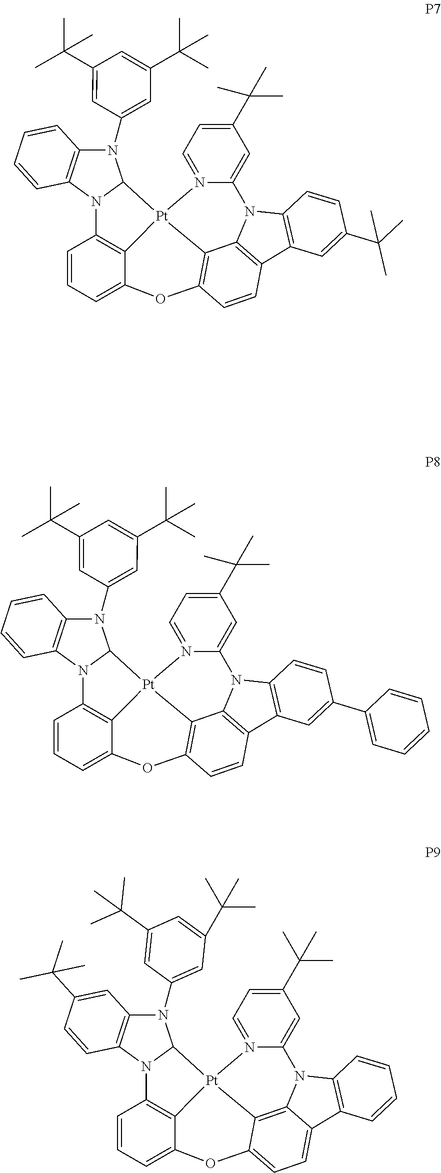

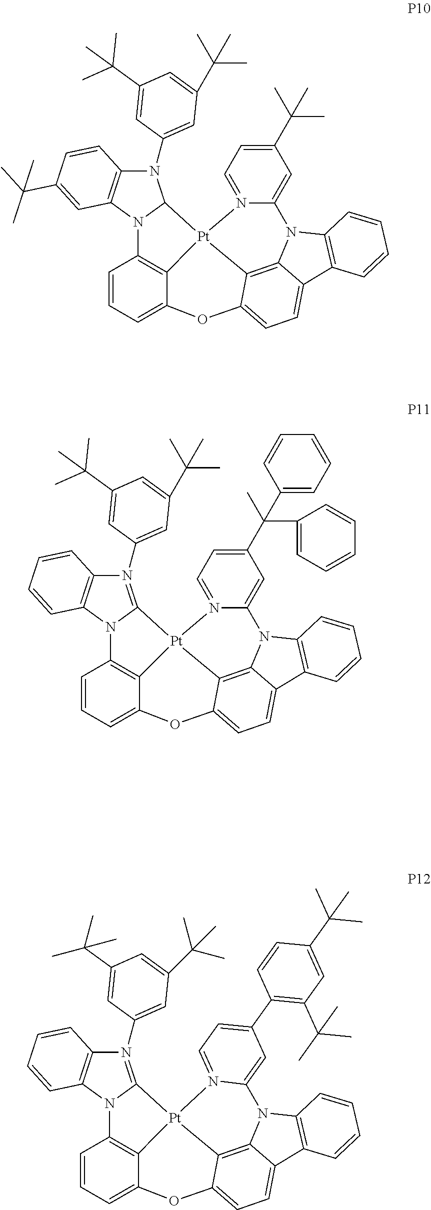

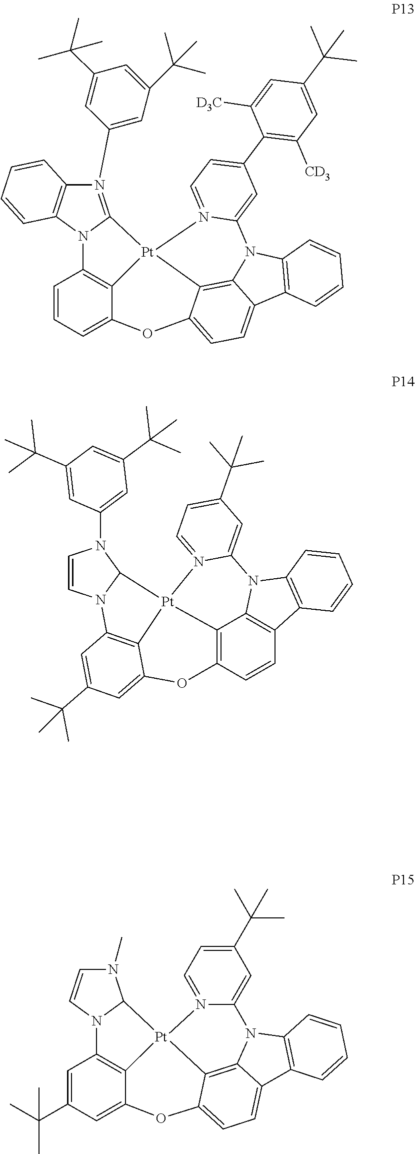

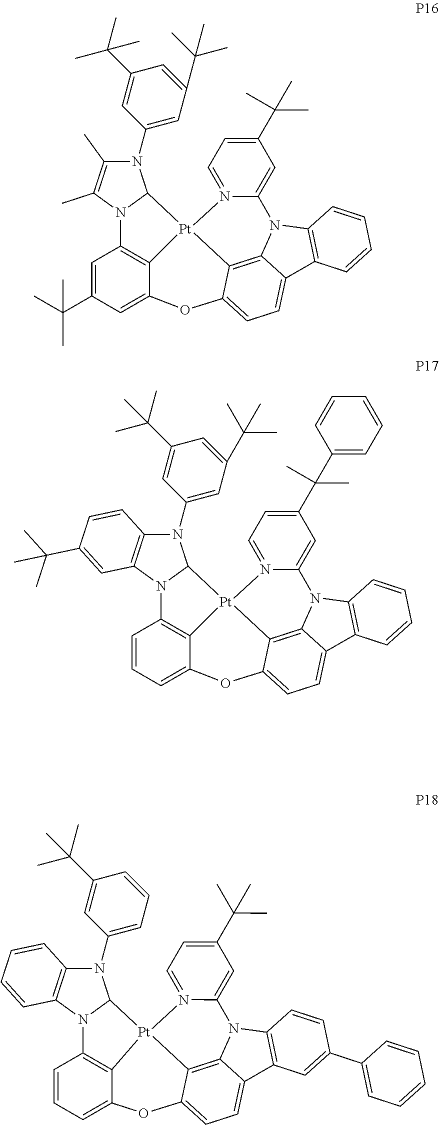

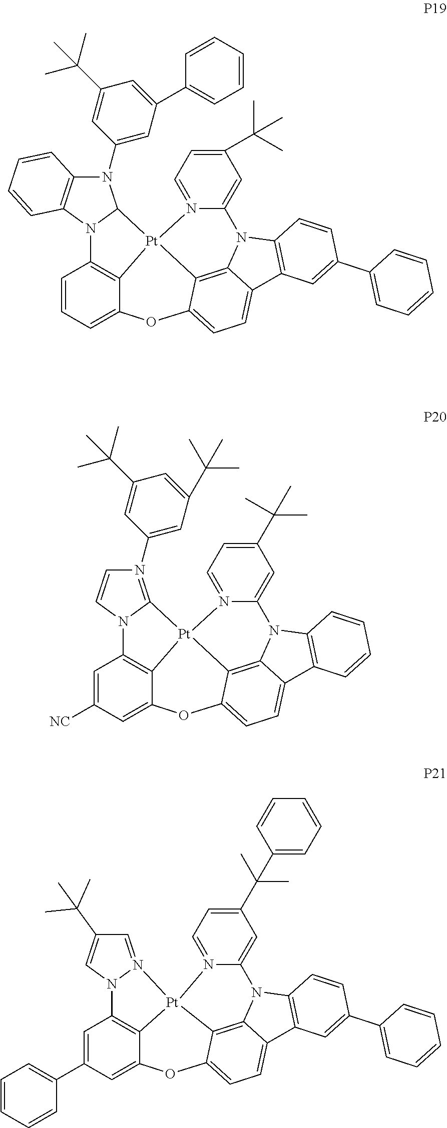

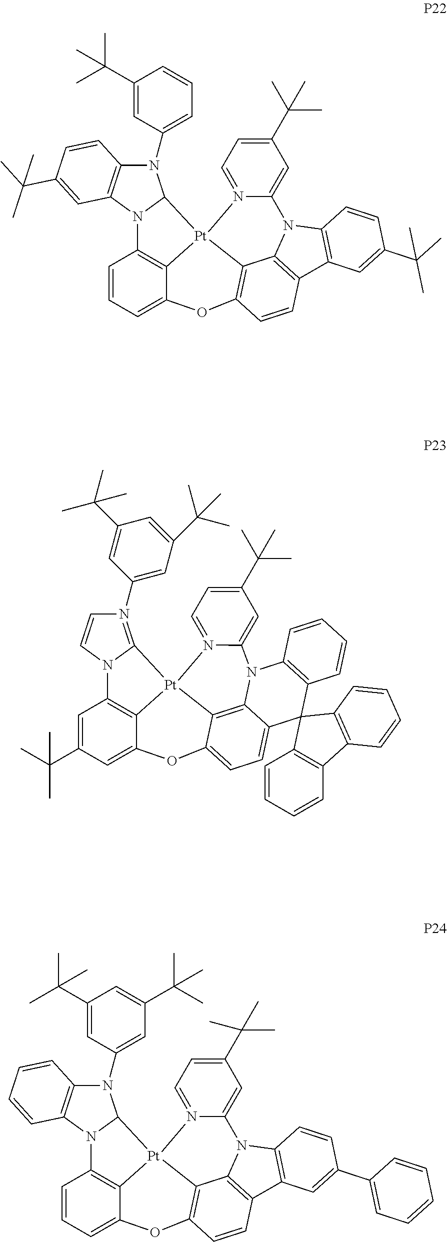

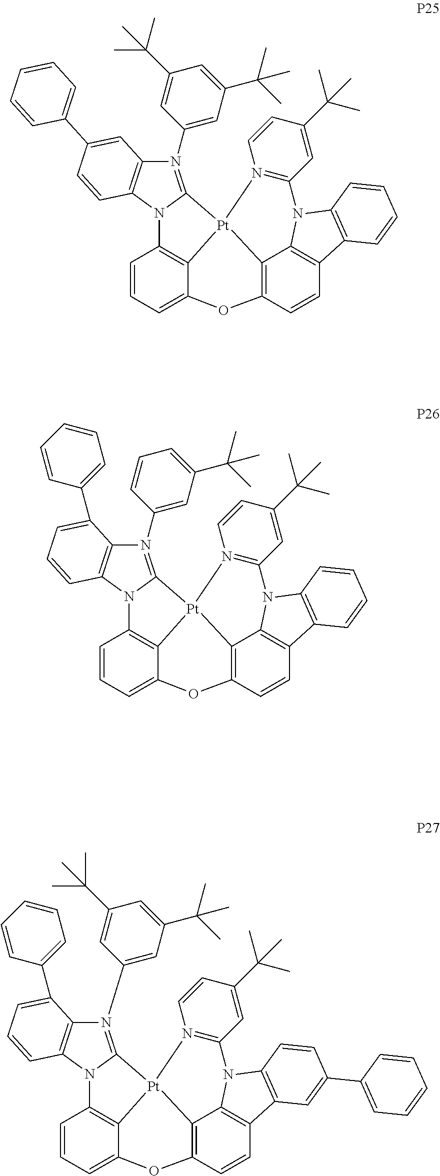

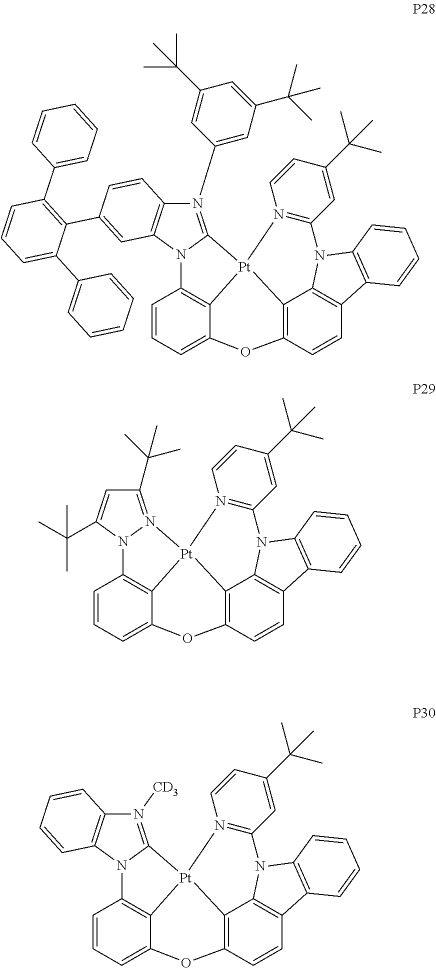

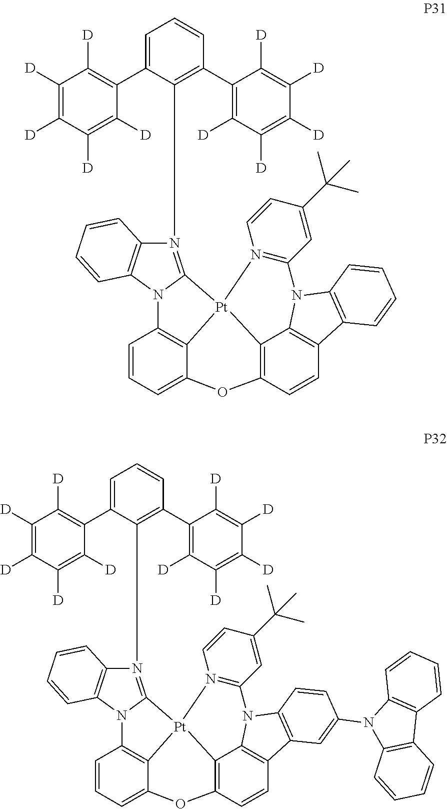

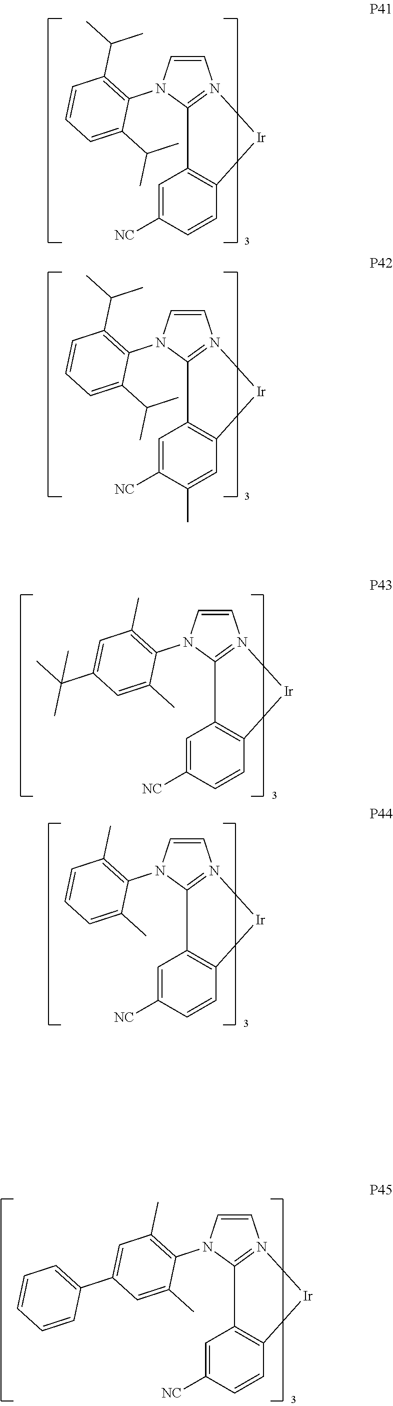

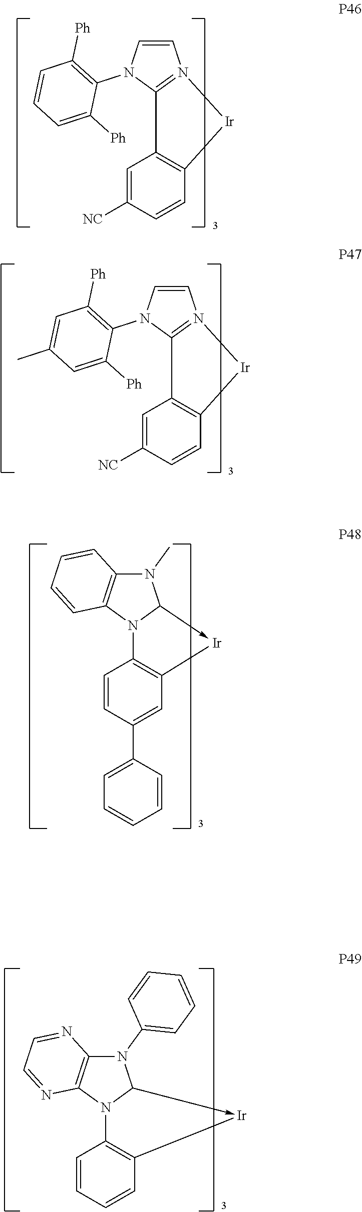

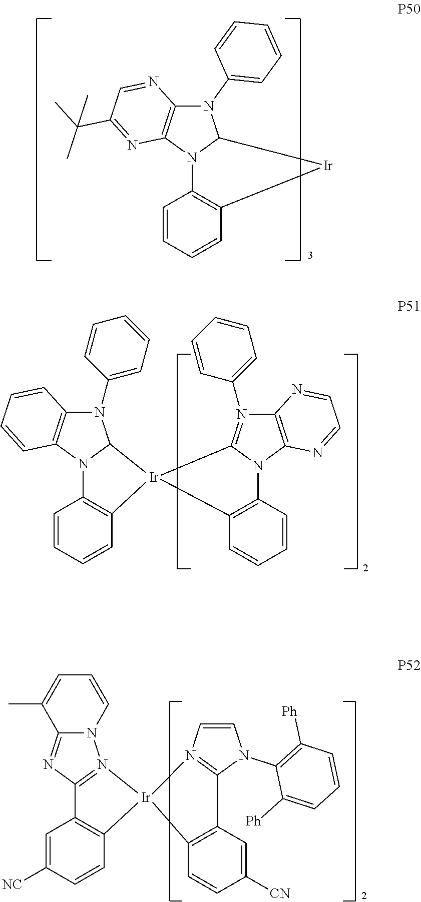

The light emitting dopant may be included, e.g., in an amount of less than or equal to about 20 wt %, and within the above range, about 0.1 wt % to about 20 wt %, about 0.1 wt % to about 15 wt %, about 0.1 wt % to about 10 wt %, about 0.1 wt % to about 7 wt %, about 0.1 wt % to about 5 wt %, about 0.1 wt % to about 4 wt %, about 1 wt % to about 20 wt %, about 1 wt % to about 15 wt %, about 1 wt % to about 10 wt %, about 1 wt % to about 7 wt %, about 1 wt % to about 5 wt %, or about 1 wt % to about 4 wt %, based on a total weight of the composition.

Hereinafter, an organic optoelectronic device using the aforementioned compound or composition is described.

The organic optoelectronic device may be, e.g., an organic light emitting diode, an organic photoelectric device, or an organic solar cell. In an implementation, the organic optoelectronic device may be an organic light emitting diode.

The organic optoelectronic device may include, e.g., an anode and a cathode facing each other, and an organic layer between the anode and the cathode, and the organic layer may include, e.g., an active layer such as a light emitting layer or a light absorbing layer, and the aforementioned compound or composition may be included in an active layer. The organic layer may include an auxiliary layer between the anode and the active layer or between the cathode and the active layer, and the aforementioned compound or composition may be included in the auxiliary layer.

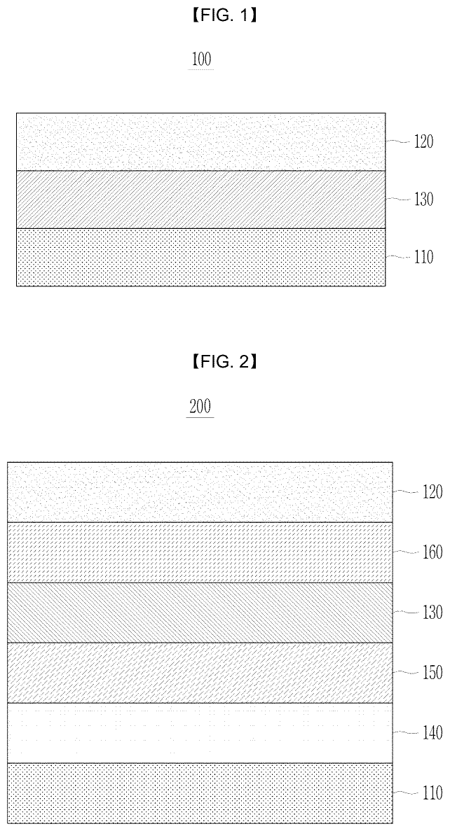

FIG. 1 is a cross-sectional view showing an example of an organic light emitting diode, which is an example of an organic optoelectronic device according to some embodiments.

Referring to FIG. 1, the organic light emitting diode 100 according to some embodiments includes, e.g., an anode 110 and a cathode 120 facing each other, and a light emitting layer 130 between the anode 110 and the cathode 120.

The anode 110 may be made of a conductor with a high work function to facilitate hole injection and may be, e.g., made of a metal, a metal oxide, or a conductive polymer. The anode 110 may be made of a metal such as nickel, platinum, vanadium, chromium, copper, zinc, gold, or an alloy thereof; a metal oxide such as zinc oxide, indium oxide, indium tin oxide (ITO), or indium zinc oxide (IZO); a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 120 may be made of a conductor with a low work function to facilitate electron injection and may be made of, e.g., a metal, a metal oxide, or a conductive polymer. The cathode 120 may be made of a metal such as magnesium, calcium, sodium, potassium, titanium, indium, yttrium, lithium, gadolinium, aluminum, silver, tin, lead, cesium, barium, or an alloy thereof; a multilayer structure material such as LiF/Al, LiO₂/Al, LiF/Ca, or BaF₂/Ca.

The light emitting layer 130 may further include the aforementioned compound or composition. The light emitting layer 130 may further include another organic compound in addition to the aforementioned compound or composition. The light emitting layer 130 may further include the light emitting dopant described above, and may include a fluorescent dopant, a phosphorescent dopant, a phosphorescent sensitizer, or a combination thereof, as described above.

In an implementation, the light emitting layer 130 may be configured to emit light in the blue emission spectrum by a combination of the aforementioned compound or composition and the light emitting dopant. A peak wavelength of the blue emission spectrum may be, e.g., in the range of about 410 nm to about 480 nm, and within the range, may be in the range of about 420 nm to about 470 nm or about 430 nm to about 470 nm. At least one of the aforementioned first and second compounds of the composition may have a high triplet energy level of greater than or equal to about 2.8 eV, so that exciton transfer to the blue light-emitting dopant may be easy, and thus an organic optoelectronic device having high-efficiency and long life-span may be realized.

In an implementation, the light emitting layer 130 may be configured to emit light in the green emission spectrum by a combination of the aforementioned compound or composition and the light emitting dopant. A peak wavelength of the green emission spectrum may be in the range of, e.g., about 500 nm to about 580 nm and within the range, may be in the range of about 510 nm to about 570 nm or about 520 nm to about 560 nm.

In an implementation, the light emitting layer 130 may be configured to emit light in the red emission spectrum by a combination of the aforementioned compound or composition and the light emitting dopant. A peak wavelength of the red emission spectrum may be in the range of, e.g., about 600 nm to about 750 nm, and within the range, may be in the range of about 610 nm to about 700 nm or about 620 nm to about 690 nm.

The aforementioned compound may effectively separate HOMO/LUMO distribution by asymmetrically arranging substituted or unsubstituted carbazolyl groups and substituted or unsubstituted biscarbazolyl groups having hole characteristics centered on a nitrogen-containing ring having electron characteristics, and thus, when the compound is applied to a device (e.g., a light emitting layer 130), life-span characteristics of the organic light emitting diode may be improved. In addition, the aforementioned compound may help lower a driving voltage of the organic light emitting diode 100 and increase the luminous efficiency by specifying the bonding position of the substituted or unsubstituted biscarbazolyl group.

In an implementation, the aforementioned composition may include the first compound, which may be a bipolar compound having relatively strong electron transport properties, and the second compound, which may be a bipolar compound having relatively strong hole transport characteristics, thereby further increasing the balance of electrons and holes within the light emitting layer 130, further helping increase luminous efficiency, and at the same time helping reduce unbound charges due to imbalance in the mobility of electrons and holes, and further helping improve the life-span of the organic light emitting diode 100.

In an implementation, an organic light emitting diode 100 including a light emitting layer 130 to which the composition is applied as a mixed host may have enhanced luminous efficiency in the light emitting layer 130 by finely controlling holes and electrons injected from the anode 110 and cathode 120 respectively to have appropriate mobility within the light emitting layer 130, thereby helping strongly induce exciton generation within the light emitting layer 130.

In an implementation, a difference in mobility of holes and electrons injected from the anode 110 and cathode 120 respectively within the light emitting layer 130 may reduce or prevent the generation of excitons at inappropriate locations, such as the interface between the light emitting layer 130 and adjacent layers, or the accumulation of unbound charges at the interface between the light emitting layer 130 and adjacent layers. Accordingly, the roll-off phenomenon in which the luminous efficiency of the organic light emitting diode 100 rapidly decreases due to non-luminescent excitons or non-bound charges may be reduced or prevented, and thus the life-span of the organic light emitting diode 100 may ultimately be improved.

The organic light emitting diode 100 may be manufactured by forming an anode 110 or a cathode 120 on a substrate, forming a light emitting layer 130 using dry film forming methods such as vacuum evaporation, sputtering, plasma plating, and ion plating, and forming a cathode 120 or an anode 110 thereon.

FIG. 2 is a cross-sectional view showing another example of an organic light emitting diode, which is an example of an organic optoelectronic device according to some embodiments.

Referring to FIG. 2, the organic light emitting diode 200 according to the present embodiments may include, e.g., an anode 110, a cathode 120, and a light emitting layer 130, similar to the aforementioned embodiments. However, unlike the aforementioned embodiments, the organic light emitting diode 200 according to the present embodiments further includes, e.g., a hole transport layer 140, a hole transport auxiliary layer 150, and an electron transport layer 160.

The hole transport layer 140 may be, e.g., between the anode 110 and the light emitting layer 130, and the hole transport auxiliary layer 150 may be, e.g., between the light emitting layer 130 and the hole transport layer 140. The electron transport layer 160 may be, e.g., between the cathode 120 and the light emitting layer 130.

The hole transport layer 140 may facilitate hole transfer from the anode 110 to the light emitting layer 130, and may include, e.g., an amine compound. In an implementation, the amine compound may have, e.g., at least one aryl group or heteroaryl group with hole characteristics. In an implementation, the amine compound may be, e.g., represented by Chemical Formula 6A or 6B.

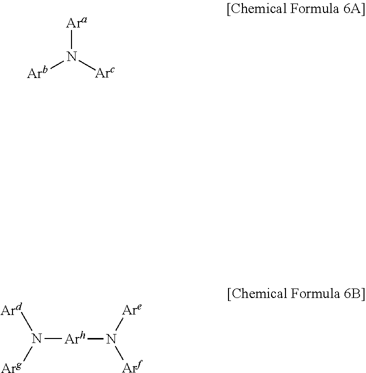

In Chemical Formula 6A or 6B, Ar^{a} to Ar^{g} may each independently be, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

At least one of Ar^{a} to Ar^{c} and at least one of Ar^{d} to Ar^{g} may be, e.g., a substituted or unsubstituted C6 to C30 aryl group, a substituted or unsubstituted C2 to C30 heteroaryl group, or a combination thereof.

Arh may be, e.g., a single bond, a substituted or unsubstituted C1 to C20 alkylene group, a substituted or unsubstituted C6 to C30 arylene group, a substituted or unsubstituted C2 to C30 heteroarylene group, or a combination thereof.

The hole transport auxiliary layer 150 may form an interface with the light emitting layer 130 by being between the hole transport layer 140 and the light emitting layer 130 and in contact with the light emitting layer 130. The hole transport auxiliary layer 150 may further reduce or prevent exciton generation at inappropriate sites, such as the interface between the aforementioned light emitting layer 130 and adjacent layers, or accumulation of unbound charges at the interface between the light emitting layer 130 and adjacent layers. Accordingly, the roll-off phenomenon in which the luminous efficiency of the organic light emitting diode 200 rapidly decreases due to non-luminescent excitons or unbound charges may be further reduced or prevented, and thus the life-span of the organic optoelectronic device 200 may be further improved.

The electron transport layer 160 may help further increase electron injection or electron mobility and block holes between the cathode 120 and the light emitting layer 130.

The electron transport layer 160 may include, e.g., a compound of Group 5.

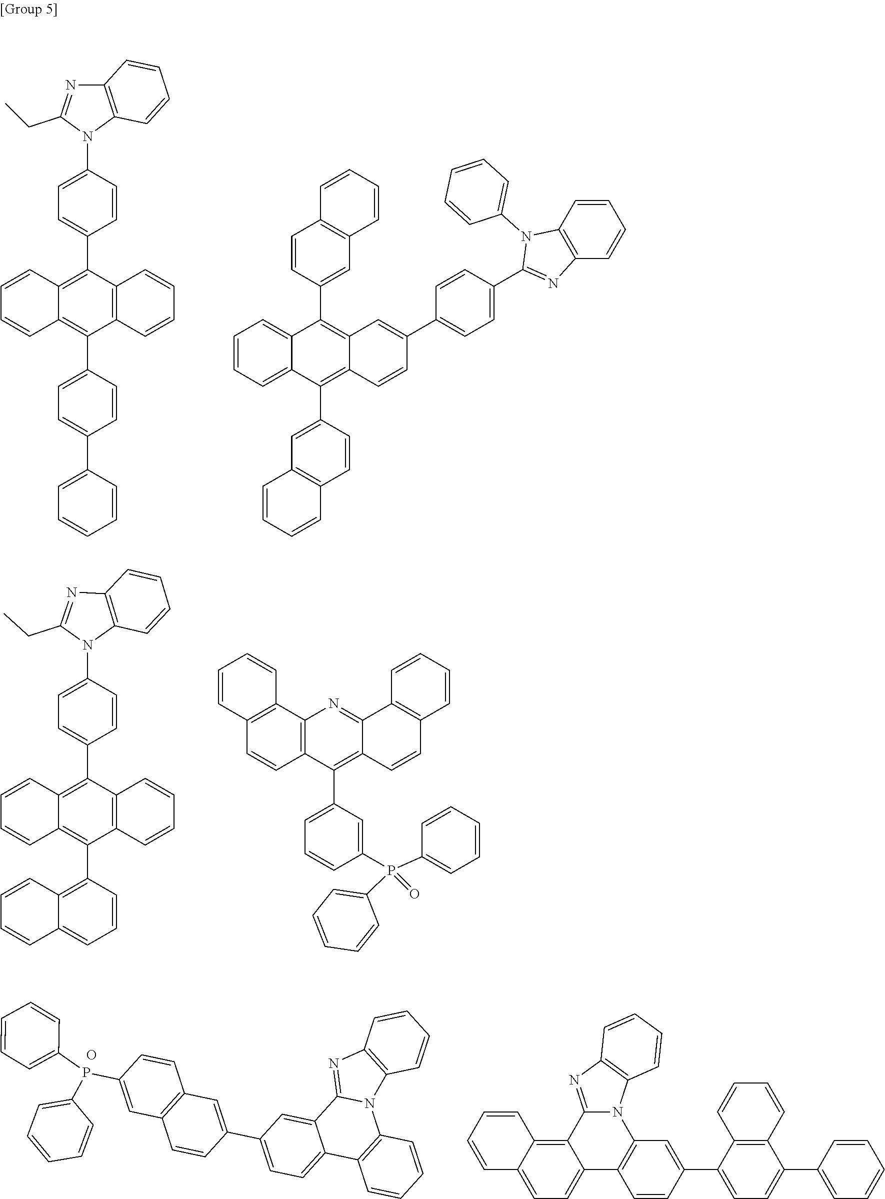

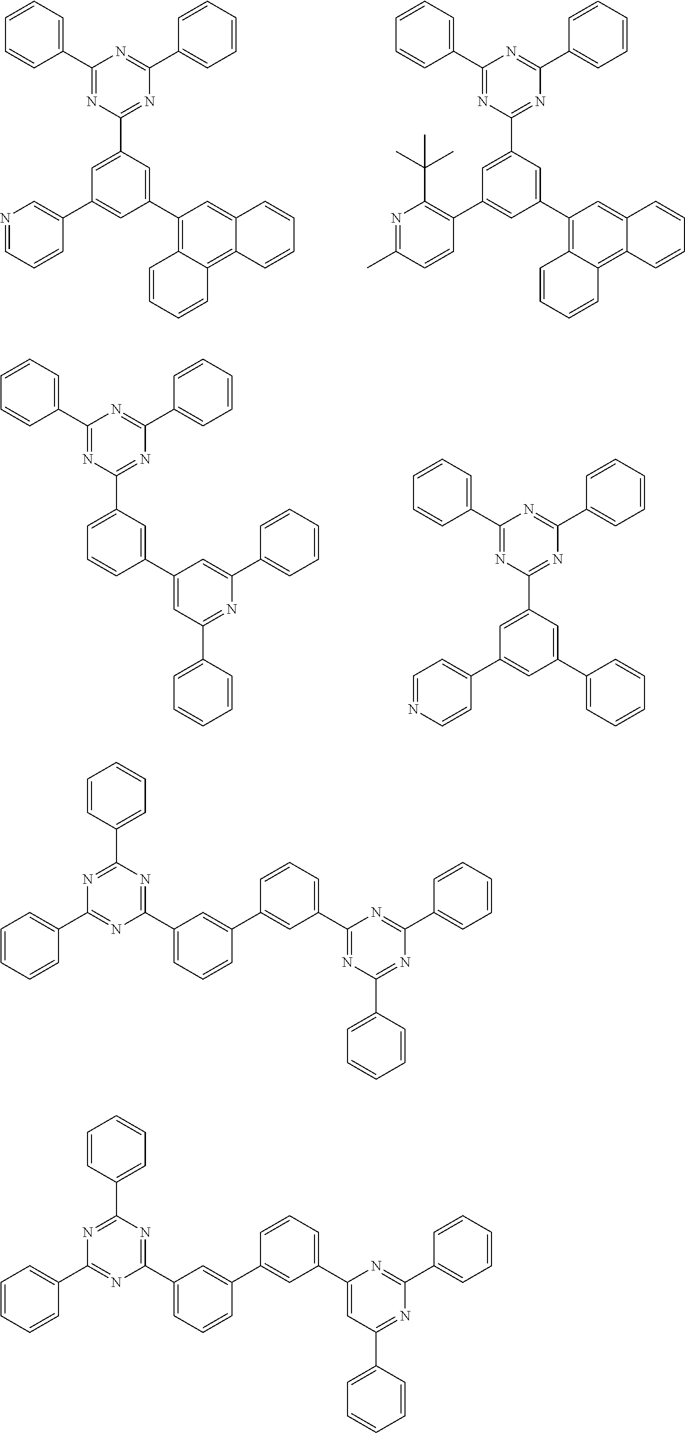 [SMILES: c1ccc(-c2nc(-c3ccccc3)nc(-c3cc(-c4cccnc4)cc(-c4cc5ccccc5c5ccccc45)c3)n2)cc1 ; Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

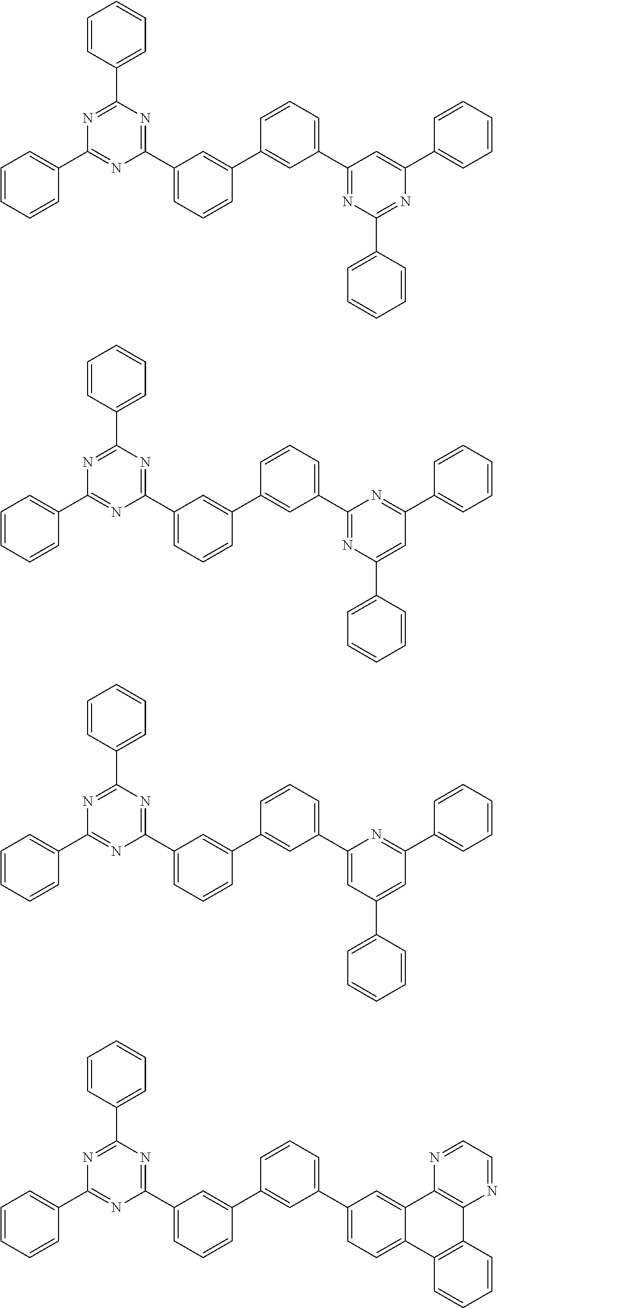 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1]

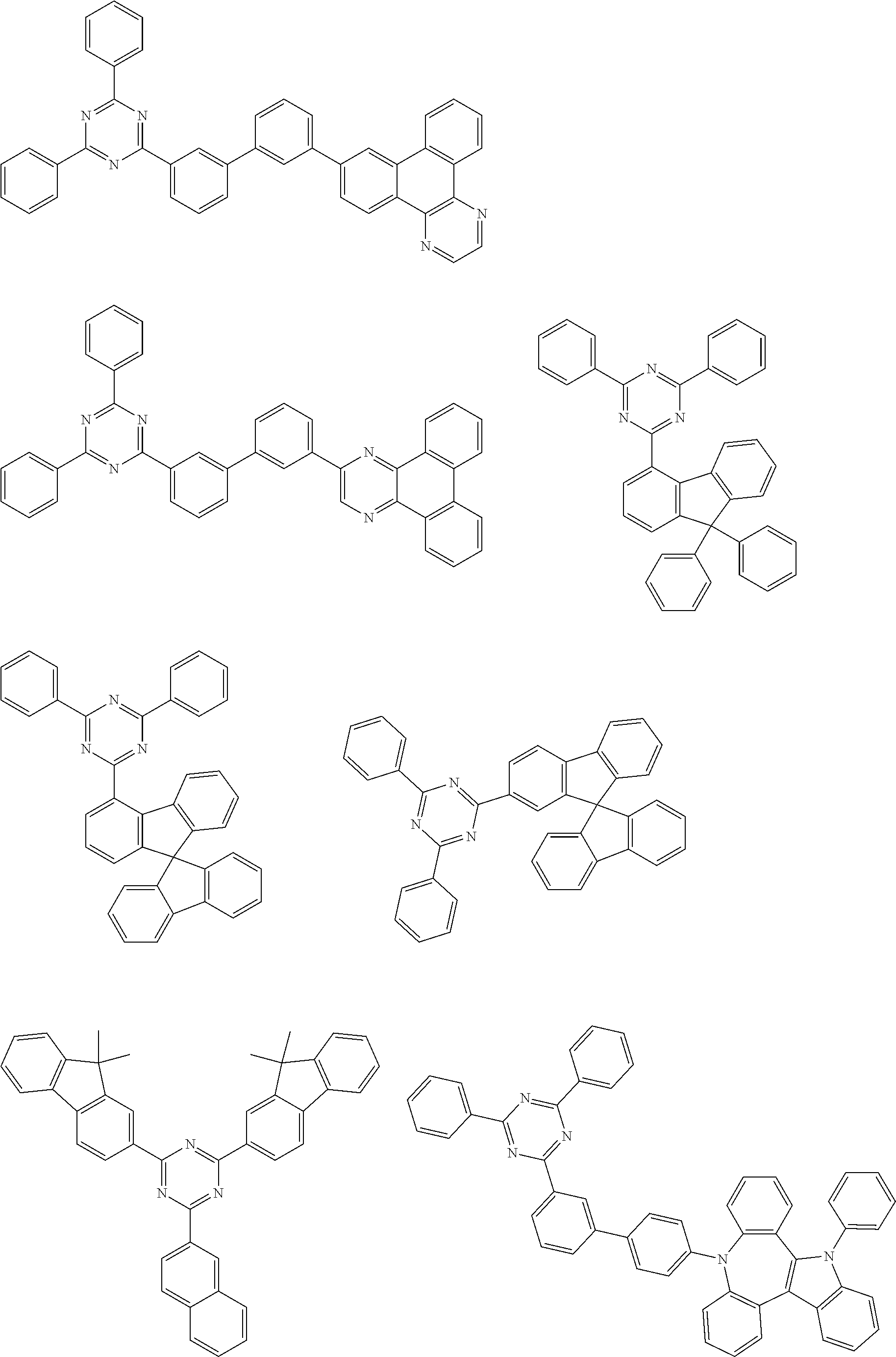

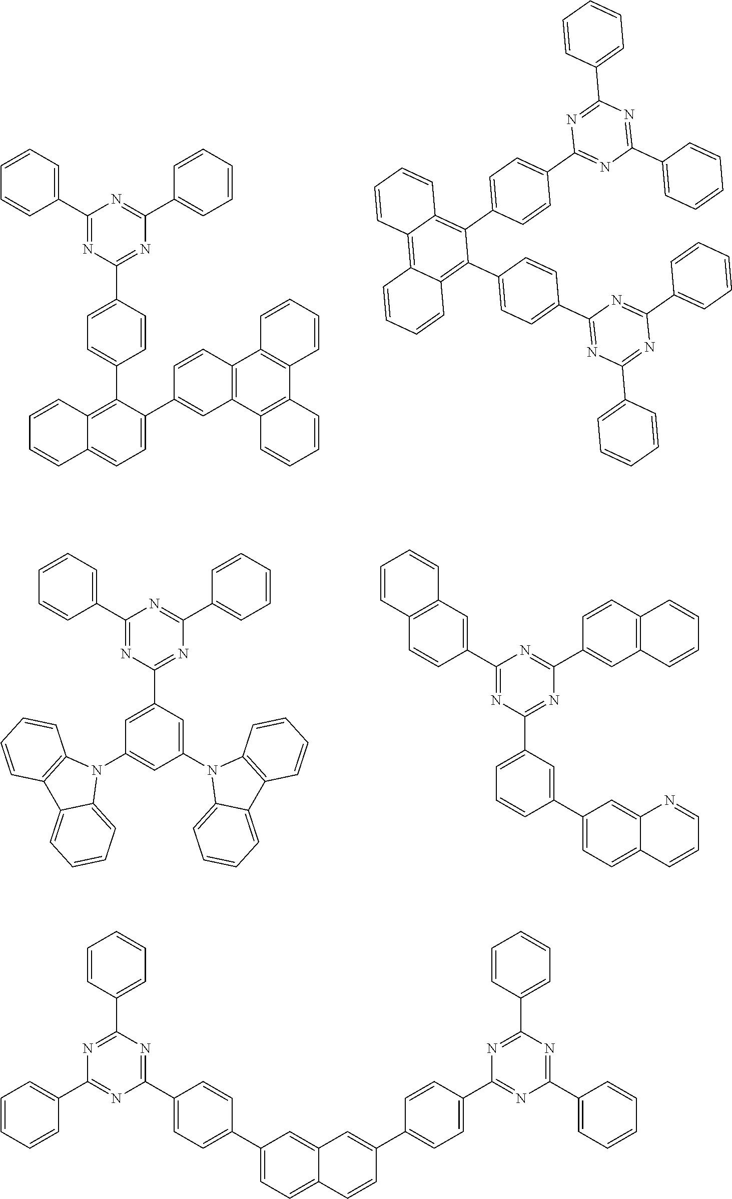

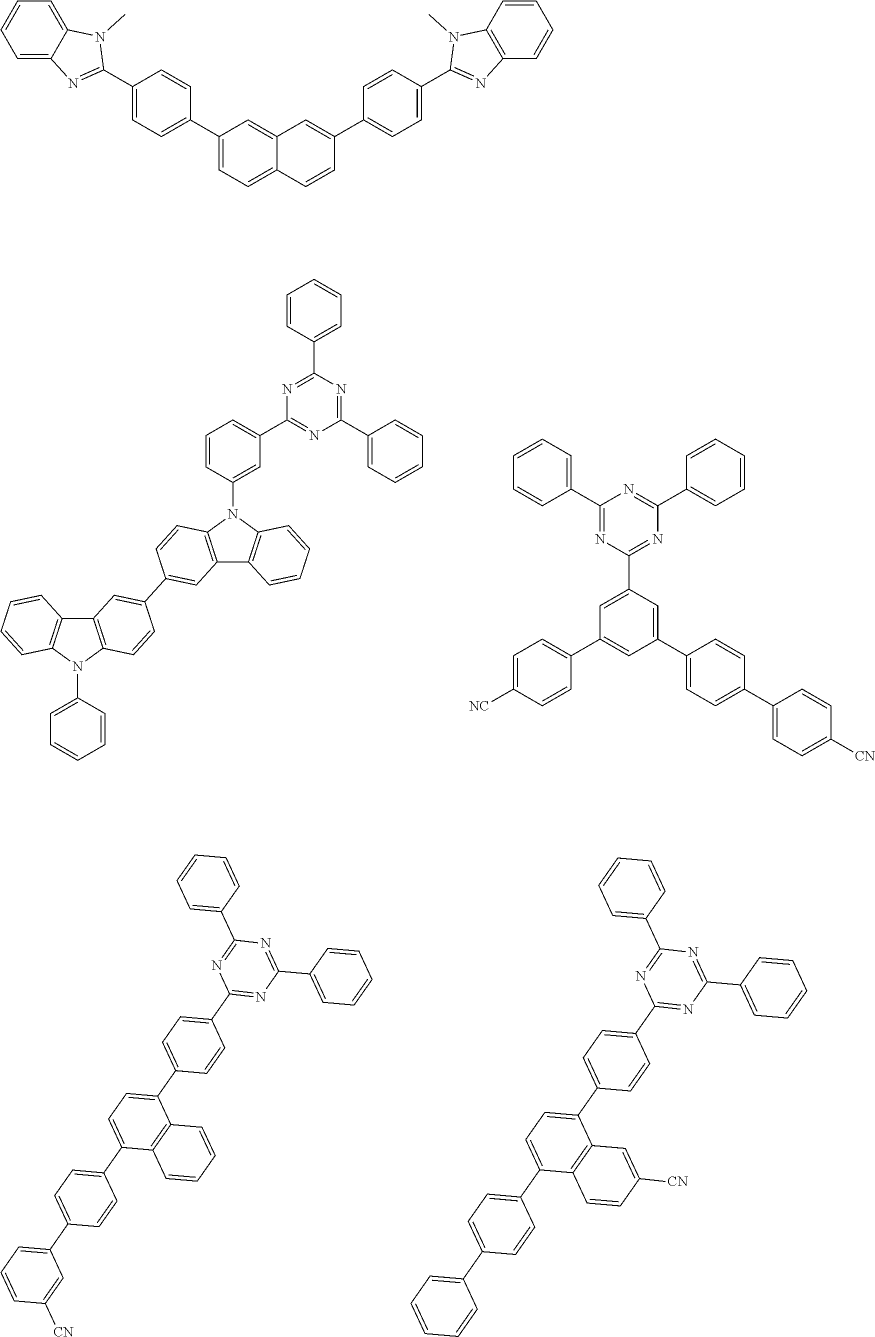 [SMILES: Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

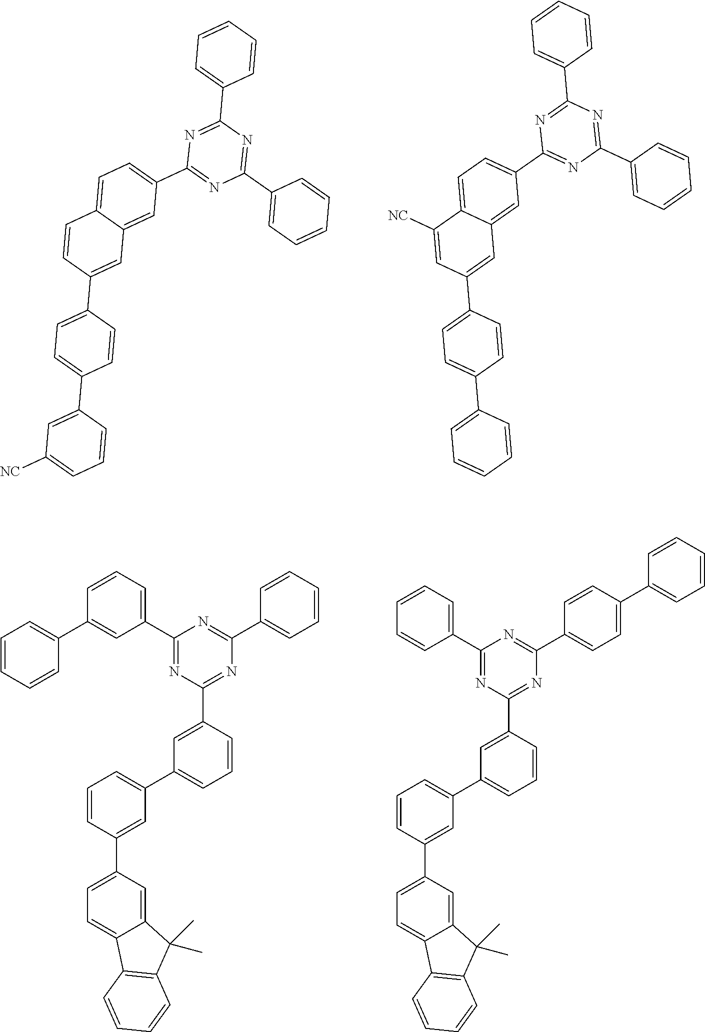 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12 ; CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

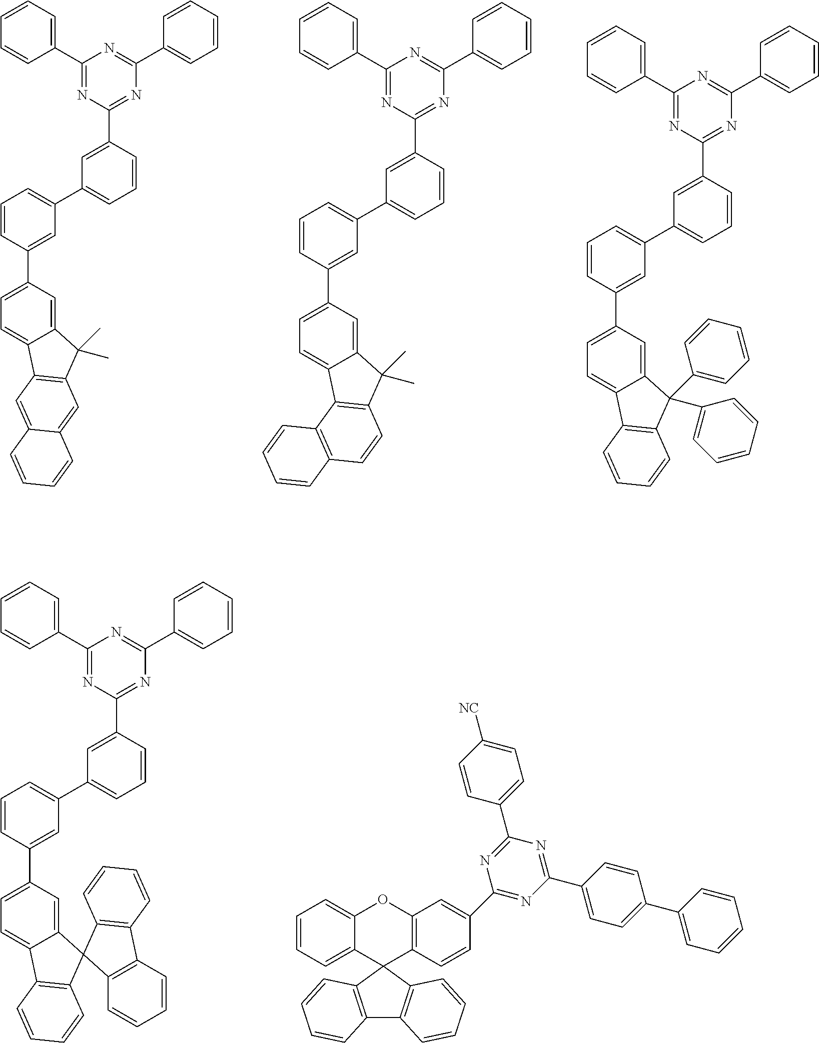 [SMILES: CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21 ; c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1 ; N#Cc1ccc(-c2nc(-c3ccc(-c4ccccc4)cc3)nc(-c3ccc4c(c3)Oc3ccccc3C43c4ccccc4-c4ccccc43)n2)cc1]

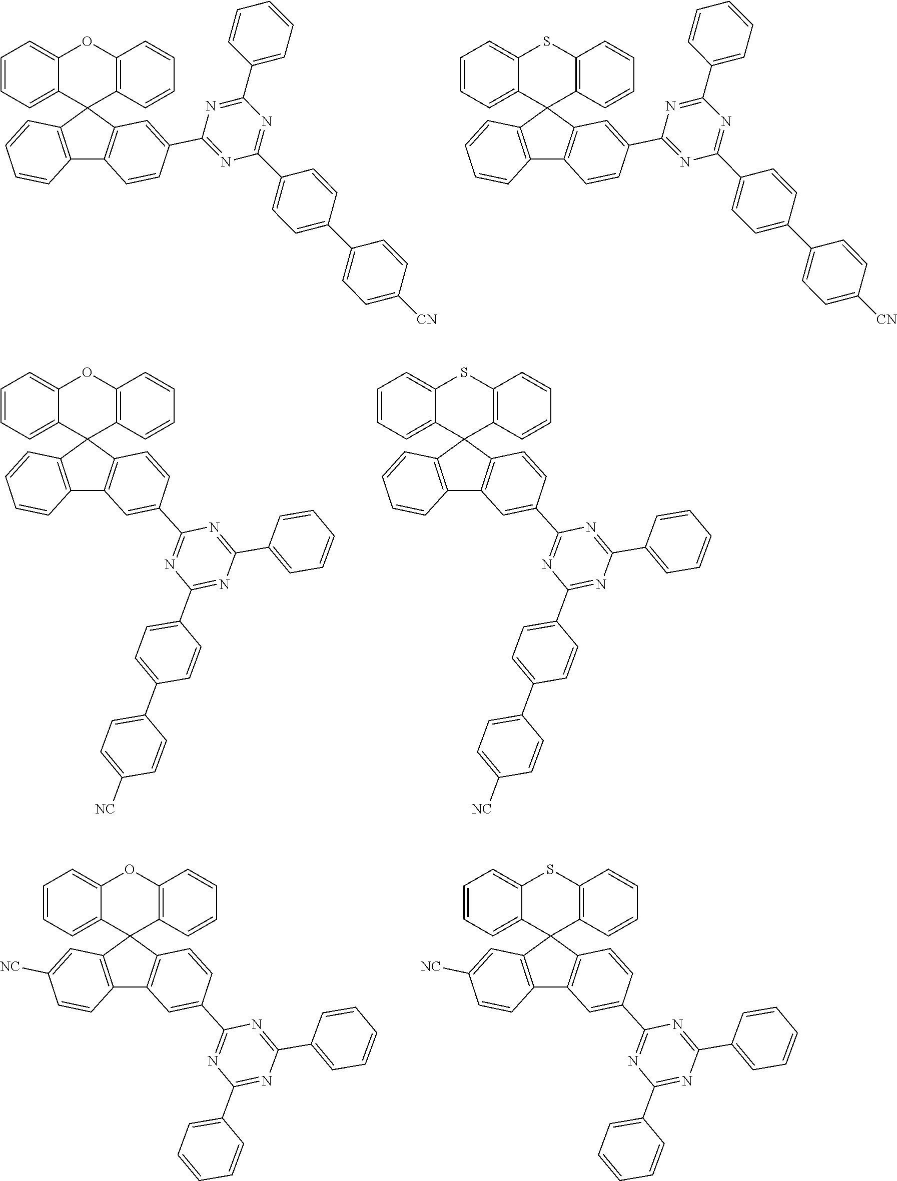

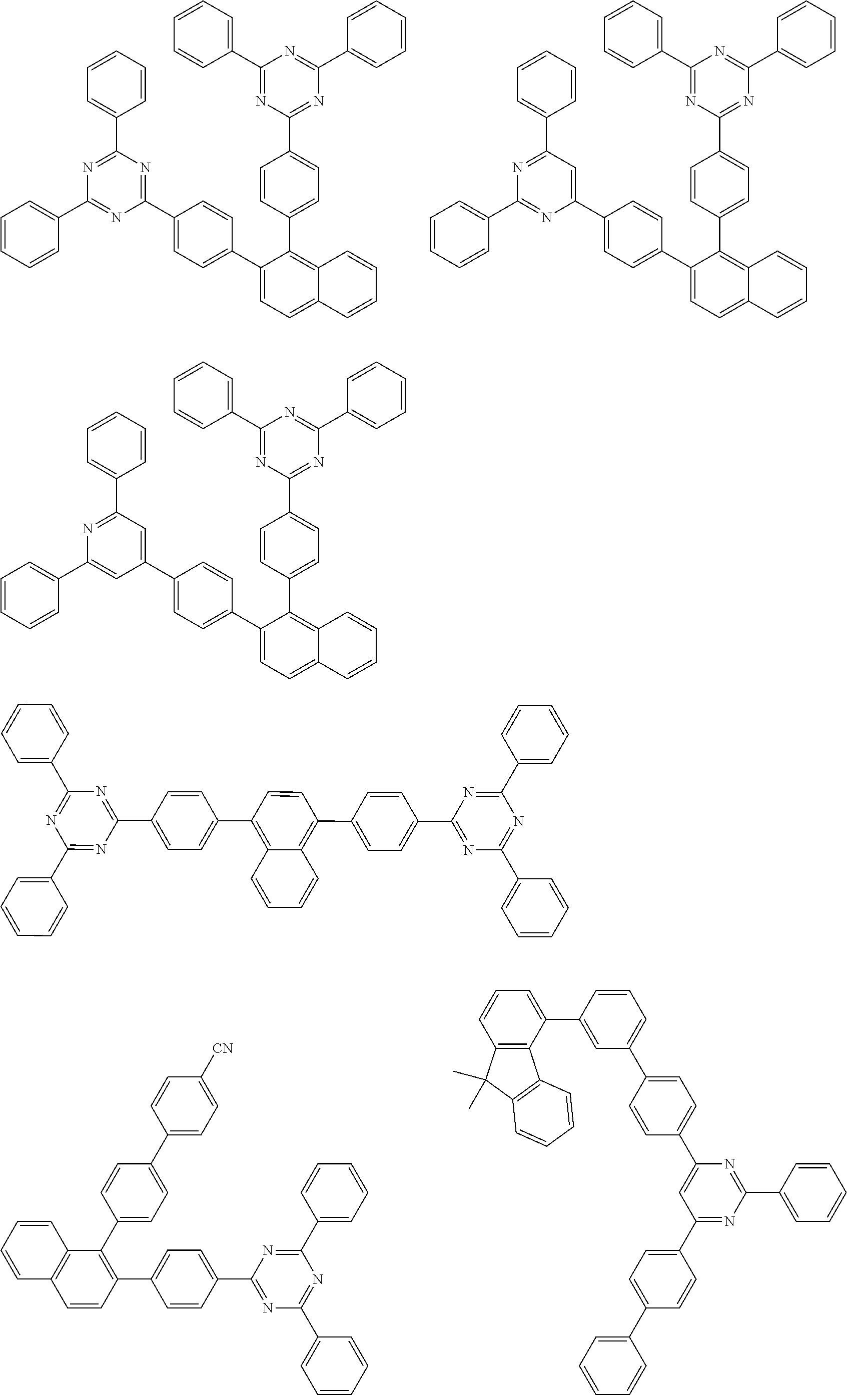

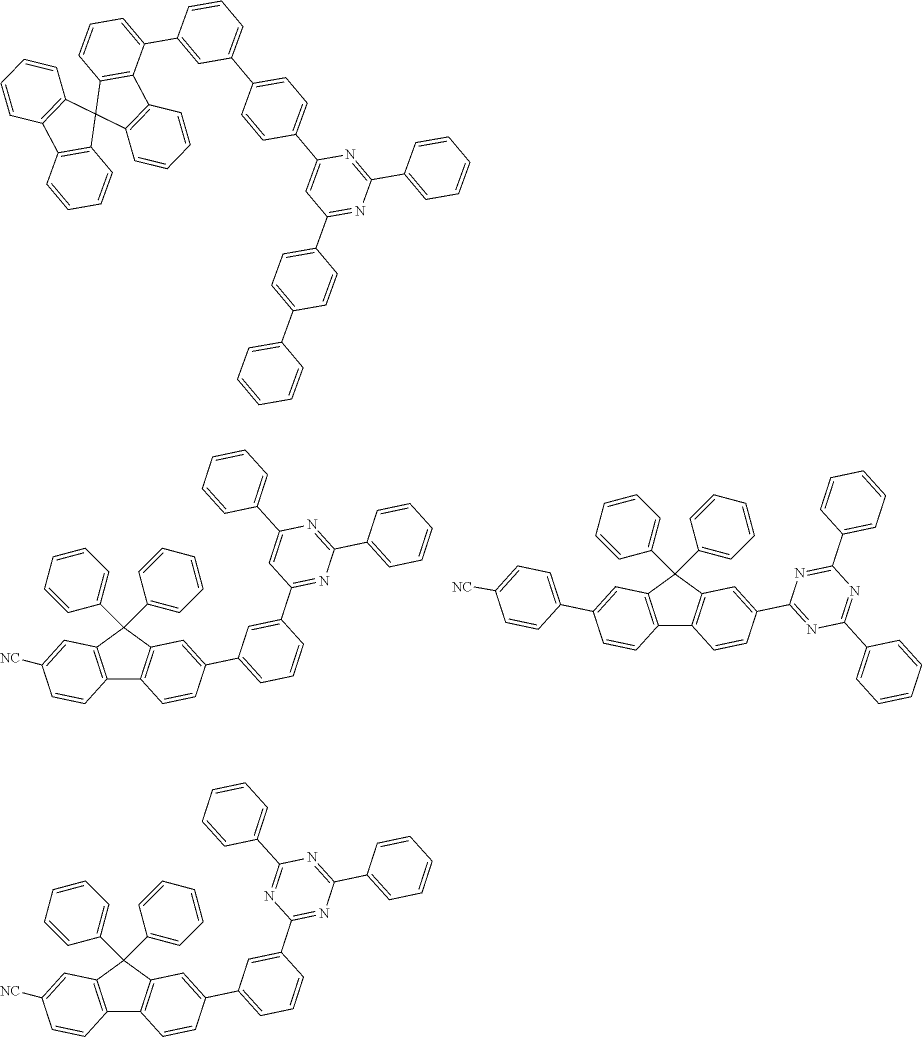 [SMILES: c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

The organic optoelectronic device, including the aforementioned organic light emitting diodes, may be applied to display devices.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry or P&H tech as far as there is no particular comment or were synthesized by suitable methods.

### Synthesis of Compounds

As a more specific example of the compound according to some embodiments, the compound presented was synthesized through the following steps.

### (Synthesis of First Compound)

### Synthesis Example 1

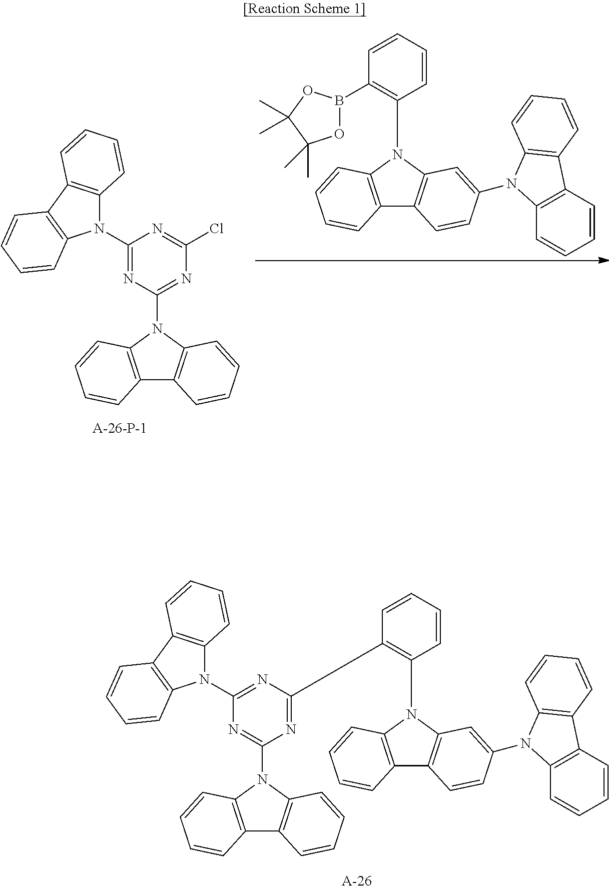

12 g of Compound A-26-P-1, 13.2 g of 9-(2-(4,4,5,5-tetramethyl-1,3,2-dioxaborolan-2-yl)phenyl)-9H-2,9′-bicarbazole, 9.1 g of K₂CO₃ (potassium carbonate), and 1.2 g of Pd (dppf) Cl₂ were added to 180 ml of a mixed solvent of 1,4-dioxane and deionized water (in a volume ratio of 2:1) and then, stirred at 75° C. for 16 hours. When a reaction was completed, after cooling the reaction mixture to ambient temperature and then, adding 500 ml of distilled water and dichloromethane (MC) thereto, work-up was performed to obtain Compound A-26.

### Synthesis Example 2

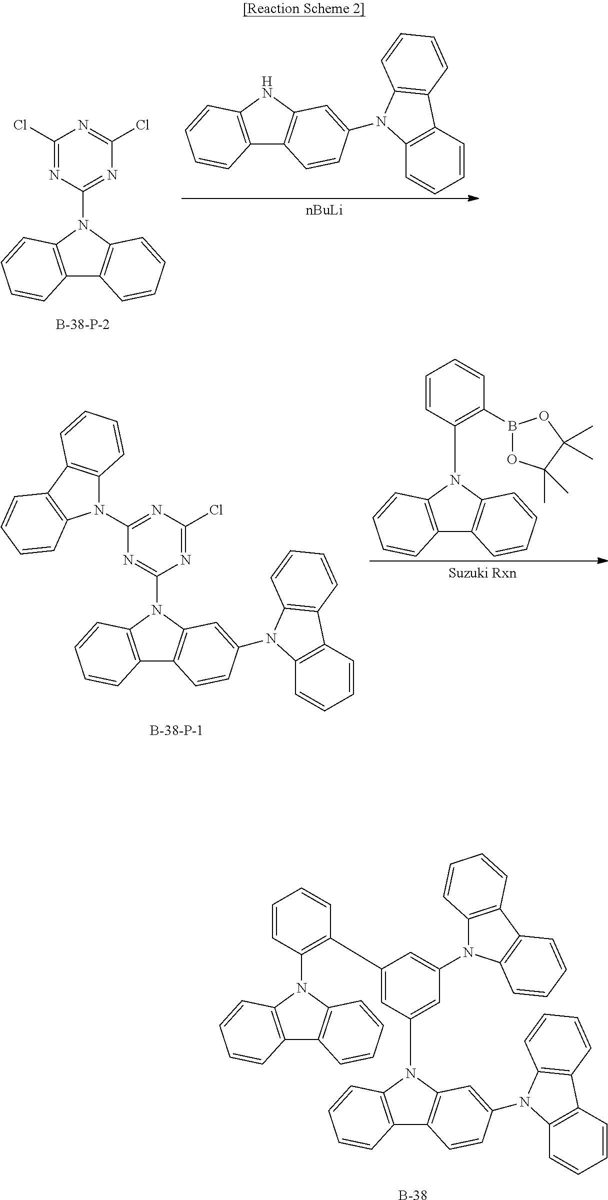

First step: Synthesis of Compound B-38-P-1

24.2 g of 9-(4,6-dichloro-1,3,5-triazine-2-yl)-9H-carbazole (Compound B-38-P-2), 20 g of 9H-2,9′-bicarbazole, 23 ml of n-BuLi (2.5 M), and 170 ml of Tetrahydrofuran (THF) were mixed and then, stirred at −78° C. for 24 hours. When a reaction was completed, the reaction mixture was added to 300 ml of distilled water at ambient temperature to produce a solid product, which was filtered and column-purified to synthesize 9-(4-(9H-carbazol-9-yl)-6-chloro-1,3,5-triazin-2-yl)-9H-2,9′-bicarbazole (Compound B-38-P-1).

### Second Step: Synthesis of Compound B-38

20 g of Compound B-38-P-1, 13.3 g of 9-(2-(4,4,5,5-tetramethyl-1,3,2-dioxaborolan-2-yl)phenyl)-9H-carbazole, 10.9 g of K₂CO₃ (potassium carbonate), and 1.3 g of Pd (dppf) Cl₂ were added to 180 ml of a mixed solvent of 1,4-dioxane and deionized water (in a volume ratio of 2:1) and then, stirred at 75° C. for 16 hours. When a reaction was completed, after cooling the reaction mixture to ambient temperature and then, adding 500 ml of distilled water and dichloromethane (MC) thereto, work-up was performed to obtain Compound B-38.

### Synthesis Example 3

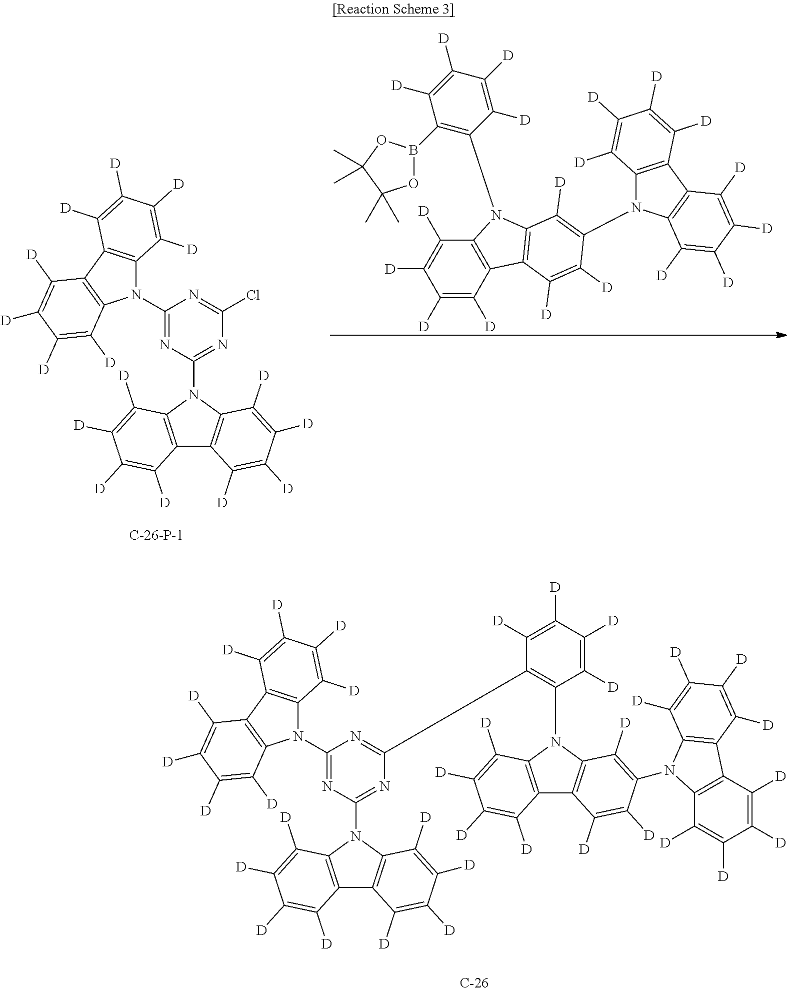

12 g of compound C-26-P-1, 13.2 g of 9-(2-(4,4,5,5-tetramethyl-1,3,2-dioxaborolan-2-yl)phenyl-3,4,5,6-d4)-9H-2,9′-bicarbazole-1,1′,2′,3,3′,4′,5,5′,6,6′,7,7′,8,8′-d14), 9.1 g of K₂CO₃, and 1.2 g of Pd (dppf) Cl₂ were added to 180 ml of a mixed solvent of 1,4-dioxane and deionized water (in a volume ratio of 2:1) and then, stirred at 75° C. for 16 hours. When a reaction was completed, after cooling the reaction mixture to ambient temperature and then, adding 500 ml of distilled water and dichloromethane (MC), work-up was performed to obtain Compound C-26.

### Synthesis Example 4

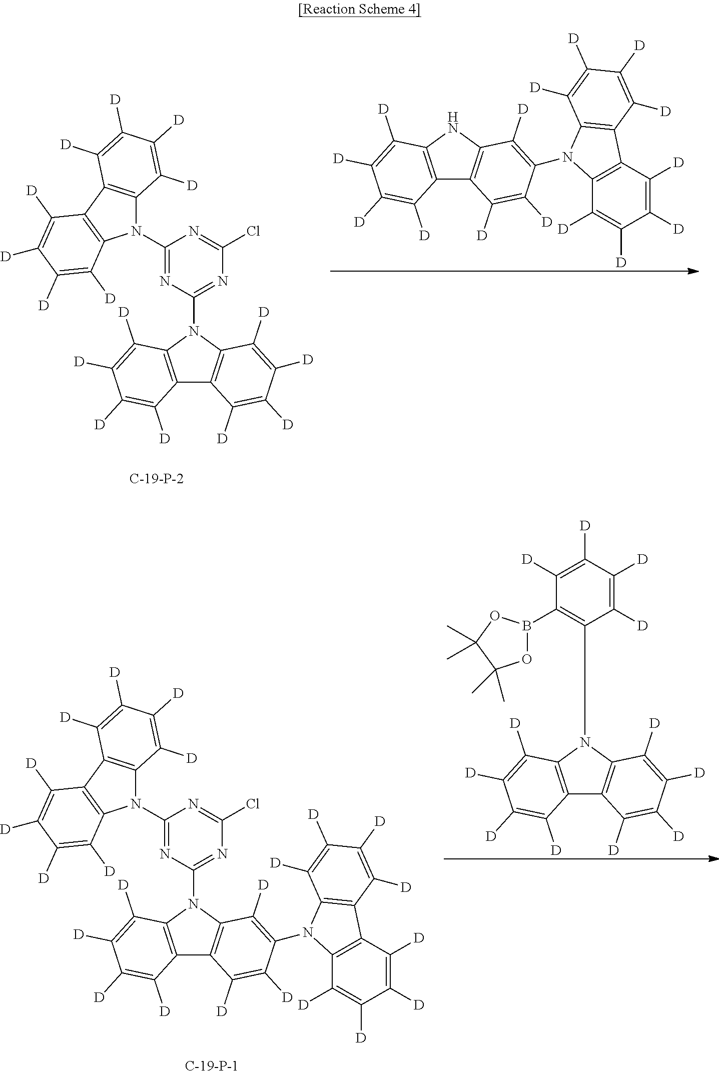

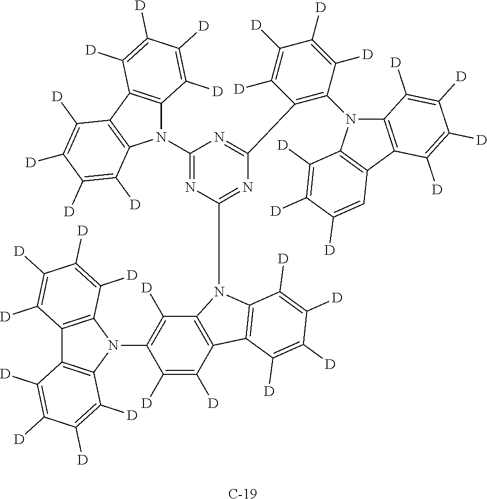

### First Step: Synthesis of Compound C-19-P-1

24.2 g of 9,9′-(6-chloro-1,3,5-triazine-2,4-diyl)bis(9H-carbazole-1,2,3,4,5,6,7,8-d8) (Compound C-19-P-2), 20 g of 9H-2,9′-bicarbazole-1,1′,2′,3,3′,4,4′,5,5′,6,6′,7,7′,8,8′-d15, 23 ml of n-BuLi (2.5 M), and 170 ml of THF were mixed and then, stirred at −78° C. for 24 hours. When a reaction was completed, the reaction mixture was added to 300 ml of distilled water at ambient temperature to produce a solid product, which was filtered and column-purified to obtain 9-(4-(9H-carbazol-9-yl-d8)-6-chloro-1,3,5-triazin-2-yl)-9H-2,9′-bicarbazole-1,1′,2′,3,3′,4,4′,5,5′,6,6′,7,7′,8,8′-d15 (Compound C-19-P-1).

### Second Step: Synthesis of Compound C-19

20 g of Compound C-19-P-1, 13.3 g of 9-(2-(4,4,5,5-tetramethyl-1,3,2-dioxaborolan-2-yl)phenyl-3,4,5,6-d4)-9H-carbazole-1,2,3,4,6,7,8-d7, 10.9 g of K₂CO₃ (potassium carbonate), and 1.3 g of Pd (dppf) Cl₂ were added to 180 ml of a mixed solvent of 1,4-dioxane and deionized water (in a volume ratio of 2:1) and then, stirred at 75° C. for 16 hours. When a reaction was completed, after cooling the reaction mixture to ambient temperature and then, adding 500 ml of distilled water and dichloromethane (MC) thereto, work-up was performed to obtain Compound C-19.

### Comparative Synthesis Example 1

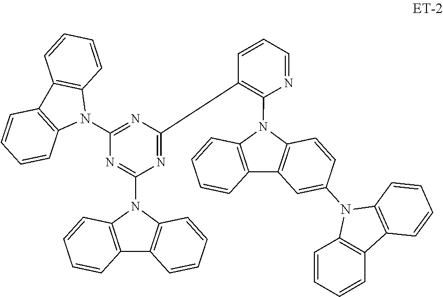 [SMILES: c1cnc(-n2c3ccccc3c3cc(-n4c5ccccc5c5ccccc54)ccc32)c(-c2nc(-n3c4ccccc4c4ccccc43)nc(-n3c4ccccc4c4ccccc43)n2)c1]

Comparative Compound ET-2 was synthesized by referring to the method disclosed in Korean Patent Publication No. KR 2023-0155974 A.

### Comparative Synthesis Example 2

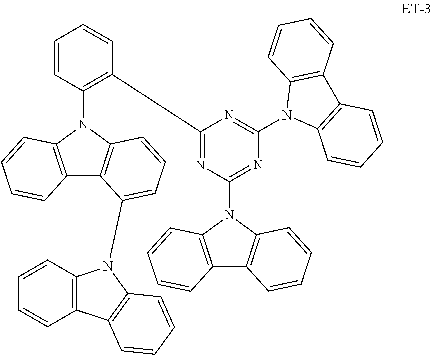 [SMILES: c1ccc(-n2c3ccccc3c3c(-n4c5ccccc5c5ccccc54)cccc32)c(-c2nc(-n3c4ccccc4c4ccccc43)nc(-n3c4ccccc4c4ccccc43)n2)c1]

Comparative Compound ET-3 was synthesized by referring to the method disclosed in Korean Patent Publication No. WO 2024/135278 A1.

### (Synthesis of Second Compound)

### Synthesis Example 5: Synthesis of Compound D-13

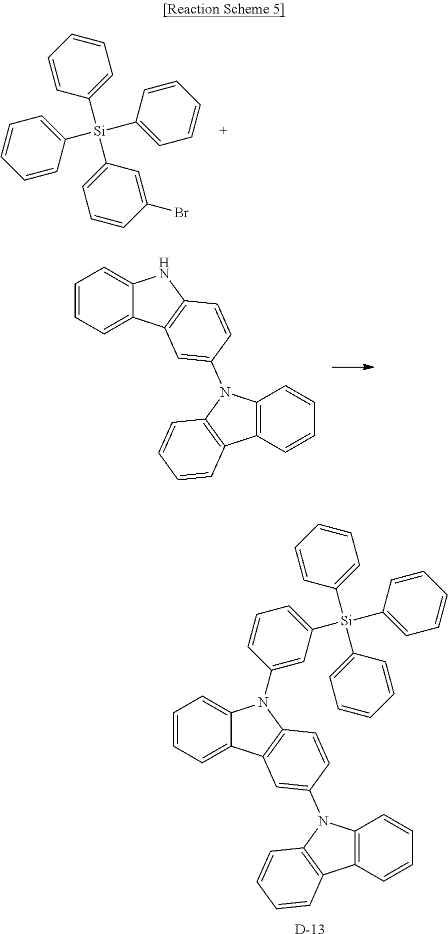

25 g of (3-bromophenyl)triphenylsilane, 30 g of 3,9′-bicarbazole, 4.1 g of Pd2(dba)3, 10.4 g of sodium t-butoxide, 6.4 ml (13.5 mmol) of tri-t-butyl phosphine solution (50% in toluene), and 300 ml of xylene were added and then, heated under reflux for 6 hours under nitrogen. The obtained mixture was extracted with water and chloridemethylene and then, purified through column chromatography to obtain 29.3 g (60.5 mmol) of Compound D-13.

LC/MS [M+H]+ 667.91

### Synthesis Example 6: Synthesis of Compound D-30

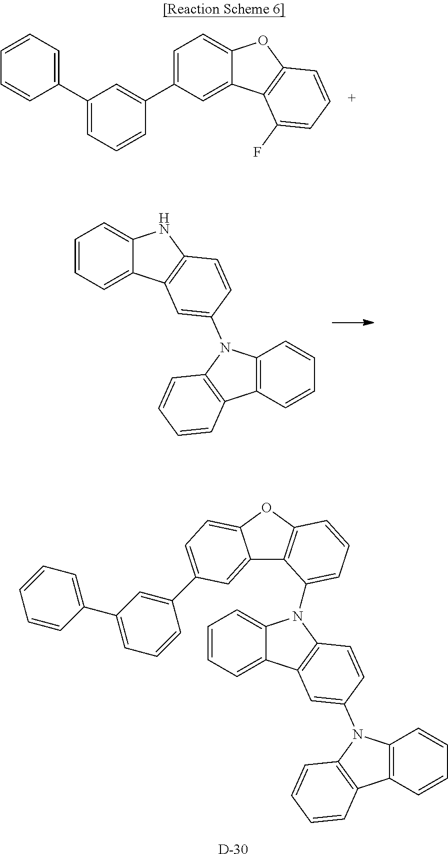

9.85 g of 8-([1,1′-biphenyl]-3-yl)-1-fluorodibenzo[b,d]furan, 11.6 g of 9H-3,9′-bicarbazole, 23.7 g of cesium carbonate, and 98 ml of N-methyl-2-pyrrolidone were added and then, stirred at 180° C. for 18 hours. After cooling to ambient temperature, water was added thereto to produce a solid, which was filtered under a reduced pressure and purified through column chromatography to obtain Compound D-30 (13.6 g, 20.9 mmol).

LC/MS [M+H]+ 651.78

### Comparative Synthesis Example 3: Synthesis of Comparative Compound H5

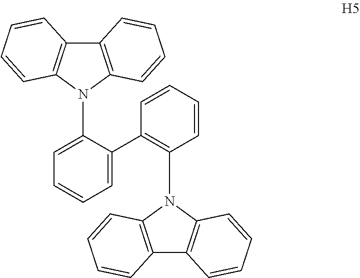 [SMILES: c1ccc(-n2c3ccccc3c3ccccc32)c(-c2ccccc2-n2c3ccccc3c3ccccc32)c1]

Comparative Compound H5 was synthesized by referring to the method disclosed in Korean Publication Patent Application No. 10-2020-0014085.

### Manufacture of Organic Light Emitting Diode I

### Example 1

A glass substrate with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor to prepare an ITO transparent electrode. The prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 600 Å to form a hole transport layer. mCP (1,3-bis(carbazol-9-yl)benzene) was deposited to a thickness of 100 Å on the hole transport layer to form a hole transport auxiliary layer. On the hole transport auxiliary layer, Compound A-26 (host) obtained in Synthesis Example 1, 13 wt % of Compound P31 (phosphorescent sensitizer), and 1.5 wt % of Compound D3 (fluorescent dopant) were vacuum-deposited to form a 400 Å-thick light emitting layer. Subsequently, BCP (2,9-dimethyl-4,7-diphenyl-1,10-phenanthroline) was deposited on the light emitting layer to a thickness of 50 Å to form an electron transport auxiliary layer, and Compound B and LiQ were simultaneously vacuum deposited in a weight ratio of 1:1 to form a 300 Å-thick electron transport layer. 10 Å of LiQ and 1,200 Å of Al were sequentially vacuum-deposited on the electron transport layer to form a cathode, manufacturing an organic light emitting diode.

### Compound A: N-(9,9-diphenyl-9H-fluoren-2-yl)-N,9-diphenyl-9H-carbazol-2-amine

### Compound B: 8-{4-[bis(naphthalene-2-yl)-1,3,5-triazin-2-yl]phenyl}quinoline

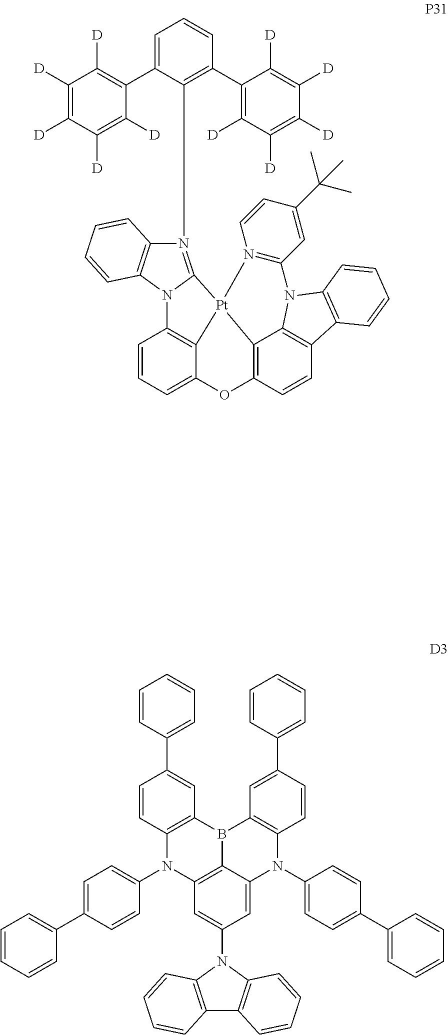

### Example 2

An organic light emitting diode was manufactured in the same manner as in Example 1, except that Compound B-38 of Synthesis Example 2 instead of Compound A-26 of Synthesis Example 1 was used as the host of the light emitting layer.

### Example 3

An organic light emitting diode was manufactured in the same manner as in Example 1, except that Compound C-26 of Synthesis Example 3 instead of Compound A-26 of Synthesis Example 1 was used as the host of the light emitting layer.

### Example 4

An organic light emitting diode was manufactured in the same manner as in Example 1, except that Compound C-19 of Synthesis Example 4 instead of Compound A-26 of Synthesis Example 1 was used as the host of the light emitting layer.

### Comparative Example 1

An organic light emitting diode was manufactured in the same manner as in Example 1, except that Compound ET-2 of Comparative Synthesis Example 1 instead of Compound A-26 of Synthesis Example 1 was used as the host of the light emitting layer.

### Comparative Example 2

An organic light emitting diode was manufactured in the same manner as in Example 1, except that Compound ET-3 of Comparative Synthesis Example 2 instead of Compound A-26 of Synthesis Example 1 was used as the host of the light emitting layer.

### Evaluation I

The organic light emitting diodes according to Examples and Comparative Examples were evaluated with respect to driving voltage, luminous efficiency, and life-span characteristics.

The specific measurement methods were as follows, and the results are shown in Tables 1 to 3.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

The luminance and the current density measured from (1) and (2) above and a voltage were used to calculate the luminous efficiency (cd/A) at the same current density (10 mA/cm²). The luminous efficiencies of the organic light emitting diodes of the examples were calculated as a relative value to that of Comparative Example 1.

### (4) Measurement of Life-span Characteristics

Time that the current efficiency (cd/A) of the organic light emitting diodes decreased to 95%, while maintaining the luminance (cd/m²) at 2000 cd/m², was measured. The measured life-spans of the organic light emitting diodes according to Examples were calculated as relative values based on Comparative Example 2.

### (5) Measurement of Driving Voltage

The driving voltage of each diode at 15 mA/cm² using a current-voltage meter (Keithley 2400) were measured to obtain the results. The measured driving voltages of the organic light emitting diodes according to Examples were calculated as relative values based on Comparative Example 2.

The results are shown in Tables 1 to 3.

TABLE 1

| | Luminous efficiency (%) |
|---|---|
| Example 1 | 115 |
| Example 2 | 110 |
| Comparative Example 1 | 100 |

TABLE 2

| | Driving voltage (%) |
|---|---|
| Example 1 | 93 |
| Example 2 | 89 |
| Comparative Example 2 | 100 |

TABLE 3

| | Life-span (%) |
|---|---|
| Example 3 | 135 |
| Example 4 | 113 |
| Comparative Example 2 | 100 |

Referring to Tables 1 to 3, the organic light emitting diodes according to Examples exhibited lower driving voltage, higher luminous efficiency, or further improved life-span characteristics compared to the organic light emitting diodes according to Comparative Examples.

### Manufacture of Organic Light Emitting Diode II

### Example 5

A glass substrate with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor to prepare an ITO transparent electrode.

The prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 600 Å to form a hole transport layer. mCP was deposited to a thickness of 100 Å on the hole transport layer to form a hole transport auxiliary layer.

On the hole transport auxiliary layer, Compound A-26 obtained in Synthesis Example 1 and Compound D-13 obtained in Synthesis Example 5 were used simultaneously as hosts, Compound P31 was doped at 13 wt % as a phosphorescent sensitizer, and Compound D3 was doped at 1.5 wt % as a fluorescent dopant to form a 400 Å-thick light emitting layer by vacuum deposition. Herein, Compound A-26 and Compound D-13 were used in a weight ratio of 5:5.

Subsequently, BCP was deposited on the light emitting layer to a thickness of 50 Å to form an electron transport auxiliary layer, and Compound B and LiQ were simultaneously vacuum deposited in a weight ratio of 1:1 to form a 300 Å-thick electron transport layer.

10 Å of LiQ and 1,200 Å of Al were sequentially vacuum-deposited on the electron transport layer to form a cathode, manufacturing an organic light emitting diode.

ITO/Compound A (3% NDP-9 doping, 100 Å)/Compound A (600 Å)/mCP (100 Å)/EML [Host (Compound A-26:Compound D-13):Compound P31:Compound D3=85.5 wt %:13 wt %:1.5 wt %] (400 Å)/BCP (50 Å)/Compound B:LiQ (300 Å)/LiQ (10 Å)/Al (1,200 Å).

### Example 6

An organic light emitting diode was manufactured in the same manner as in Example 5, except that Compound B-38 of Synthesis Example 2 and Compound D-13 of Synthesis Example 5 were used instead of Compound A-26 of Synthesis Example 1 and Compound D-13 of Synthesis Example 5 as the host of the light emitting layer.

### Example 7

An organic light emitting diode was manufactured in the same manner as in Example 5, except that Compound C-26 of Synthesis Example 3 and Compound D-13 of Synthesis Example 5 were used instead of Compound A-26 of Synthesis Example 1 and Compound D-13 of Synthesis Example 5 as the host of the light emitting layer.

### Example 8

An organic light emitting diode was manufactured in the same manner as in Example 5, except that Compound C-19 of Synthesis Example 4 and Compound D-13 of Synthesis Example 5 were used instead of Compound A-26 of Synthesis Example 1 and Compound D-13 of Synthesis Example 5 as the host of the light emitting layer.

### Comparative Example 3

An organic light emitting diode was manufactured in the same manner as in Example 5, except that Compound ET-2 of Comparative Synthesis Example 1 and Compound D-13 of Synthesis Example 5 were used instead of Compound A-26 of Synthesis Example 1 and Compound D-13 of Synthesis Example 5 as the host of the light emitting layer.

### Comparative Example 4

An organic light emitting diode was manufactured in the same manner as in Example 5, except that Compound ET-3 of Comparative Synthesis Example 2 and Compound D-13 of Synthesis Example 5 were used instead of Compound A-26 of Synthesis Example 1 and Compound D-13 of Synthesis Example 5 as the host of the light emitting layer.

### Evaluation II

The organic light emitting diodes according to Examples and Comparative Examples were evaluated with respect to luminous efficiency, driving voltage, and life-span characteristics according to the measurement methods specified in Evaluation I. The luminous efficiencies of the organic light emitting diodes were calculated as relative values based on Comparative Example 3 and the measured driving voltages and life-span characteristics were calculated as relative values based on Comparative Example 4. The results are shown in Tables 4 to 6.

TABLE 4

| | Host | | Luminous |
|---|---|---|---|
| | First | Second | efficiency |
| No. | compound | compound | (%) |
| Example 5 | A-26 | D-13 | 127 |
| Example 6 | B-38 | D-13 | 122 |
| Comparative Example 3 | ET-2 | D-13 | 100 |

TABLE 5

| | Host | | Driving |
|---|---|---|---|
| | First | Second | voltage |
| No. | compound | compound | (%) |
| Example 5 | A-26 | D-13 | 93 |
| Example 6 | B-38 | D-13 | 91 |
| Comparative Example 4 | ET-3 | D-13 | 100 |

TABLE 6

| | Host | | |
|---|---|---|---|
| | First | Second | Life- |
| No. | compound | compound | span (%) |
| Example 7 | C-26 | D-13 | 189 |
| Example 8 | C-19 | D-13 | 120 |
| Comparative Example 4 | ET-3 | D-13 | 100 |

Referring to Tables 4 to 6, the organic light emitting diodes according to Examples exhibited lower driving voltage, higher luminous efficiency, or further improved life-span characteristics compared to the organic light emitting diodes according to Comparative Examples.

By way of summation and review, some example embodiments may provide a compound for an organic optoelectronic device that may achieve high efficiency and long life-span characteristics. Some example embodiments may provide a composition for an organic optoelectronic device including the compound for an organic optoelectronic device. Some example embodiments may provide an organic optoelectronic device including the compound or the composition. Some example embodiments may provide a display device including the organic optoelectronic device.

An organic optoelectronic device having high efficiency and a long life-span may be realized through come of the example embodiments.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound represented by Chemical Formula 1: 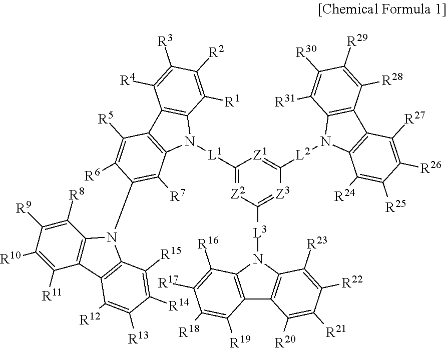
wherein, in Chemical Formula 1,
Z¹ to Z³ are each independently N or CR^{a}, provided that at least two of Z¹ to Z³ are N,
L¹ to L³ are each independently a single bond or a substituted or unsubstituted C6 to C20 arylene group, provided that one or two of L¹ to L³ are a substituted or unsubstituted o-phenylene group or a substituted or unsubstituted m-phenylene group, and
R¹ to R³¹ and R^{a} are each independently hydrogen, deuterium, a substituted or unsubstituted C1 to C30 alkyl group, a substituted or unsubstituted C6 to C30 aryl group, a substituted or unsubstituted C2 to C30 heterocyclic group, excluding a carbazolyl group, a substituted or unsubstituted silyl group, a substituted or unsubstituted amine group, a halogen, a cyano group, or a combination thereof.

2. The compound as claimed in claim 1, wherein:
one or two of L¹ to L³ are a substituted or unsubstituted o-phenylene group, and
the remaining one or two of L¹ to L³ are single bonds.

3. The compound as claimed in claim 1, wherein the compound is represented by Chemical Formula 1A or 1B: 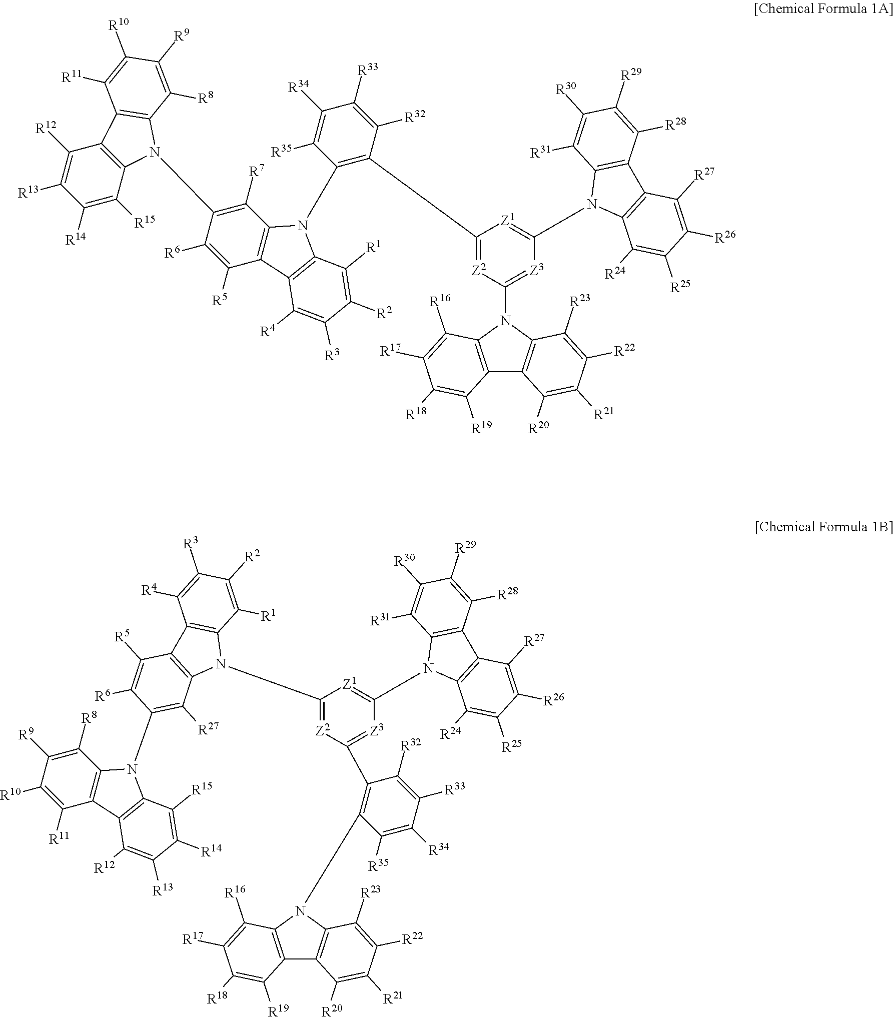
wherein, in Chemical Formula 1A or 1B,
Z¹ to Z³ are each independently N or CR^{a}, provided that at least two of Z¹ to Z³ are N, and
R¹ to R³⁵ and R^{a} are each independently hydrogen, deuterium, a substituted or unsubstituted C1 to C30 alkyl group, a substituted or unsubstituted C6 to C30 aryl group, a substituted or unsubstituted C2 to C30 heterocyclic group, excluding a carbazolyl group, a substituted or unsubstituted silyl group, a substituted or unsubstituted amine group, a halogen, a cyano group, or a combination thereof.

4. A composition, the composition comprising:
a first compound, the first compound being the compound claimed in claim 1, and
a second compound, the second compound being different from the first compound and including at least one carbazole moiety.

5. The composition as claimed in claim 4, wherein the second compound is represented by Chemical Formula 2: 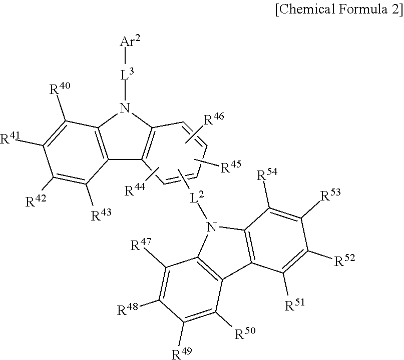
wherein, in Chemical Formula 2,
L⁴ and L⁵ are each independently a single bond, a substituted or unsubstituted C6 to C30 arylene group, a substituted or unsubstituted divalent C3 to C30 heterocyclic group, or a combination thereof,
Ar² is a substituted or unsubstituted C6 to C30 aryl group or a substituted or unsubstituted C2 to C30 heterocyclic group,
R⁴⁰ to R⁵⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
R⁴⁰ to R⁵⁴ are each separately present or two adjacent ones are linked to each other to form a ring.

6. The composition as claimed in claim 5, wherein:
L⁴ and L⁵ are each independently a single bond, a substituted or unsubstituted phenylene group, a substituted or unsubstituted biphenylene group, or a substituted or unsubstituted terphenylene group, and
Ar² is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

7. The composition as claimed in claim 5, wherein at least one of Ar² and R⁴⁰ to R⁵⁴ is a substituted or unsubstituted carbazolyl group.

8. The composition as claimed in claim 4, wherein:
the second compound is represented by Chemical Formula 3: 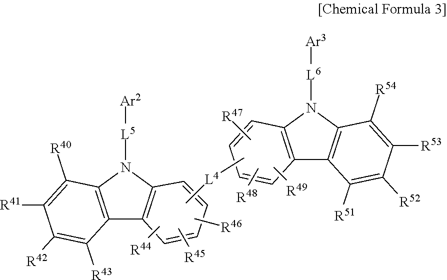
in Chemical Formula 3,
L⁴ to L⁶ are each independently a single bond, a substituted or unsubstituted C6 to C30 arylene group, a substituted or unsubstituted divalent C3 to C30 heterocyclic group, or a combination thereof,
Ar² and Ar³ are each independently a substituted or unsubstituted C6 to C30 aryl group or a substituted or unsubstituted C2 to C30 heterocyclic group,
R⁴⁰ to R⁴⁹ and R⁵¹ to R⁵⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
R⁴⁰ to R⁴⁹ and R⁵¹ to R⁵⁴ are each separately present or two adjacent ones are linked to each other to form a ring.

9. The composition as claimed in claim 8, wherein:
L⁴ to L⁶ are each independently a single bond, a substituted or unsubstituted phenylene group, a substituted or unsubstituted biphenylene group, or a substituted or unsubstituted terphenylene group, and
Ar² and Ar³ are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

10. The composition as claimed in claim 4, wherein:
the second compound is represented by a combination of Chemical Formulas 4 and 5: 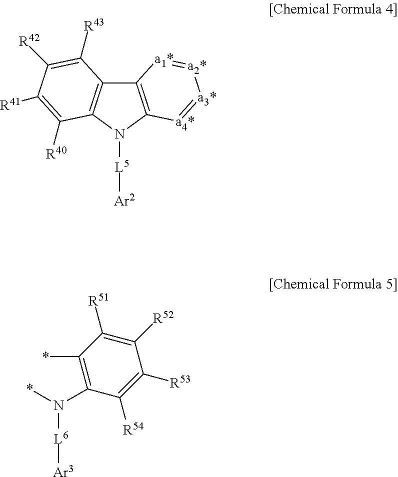
in Chemical Formulas 4 and 5,
two adjacent ones of a₁* to a₄* of Chemical Formula 4 are each linking carbons linked at * of Chemical Formula 5,
the remaining two of a₁* to a₄* of Chemical Formula 4, not linked at * of Chemical Formula 5, are each independently C-L^{b}-R^{b},
L⁵, L⁶, and L^{b} are each independently a single bond, a substituted or unsubstituted C6 to C30 arylene group, a substituted or unsubstituted divalent C3 to C30 heterocyclic group, or a combination thereof,
Ar² and Ar³ are each independently a substituted or unsubstituted C6 to C30 aryl group or a substituted or unsubstituted C2 to C30 heterocyclic group,
R⁴⁰ to R⁴³, R⁵¹ to R⁵⁴, and R^{b} are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, and
R⁴⁰ to R⁴³, R⁵¹ to R⁵⁴, and R^{b} are each separately present or two adjacent ones are linked to each other to form a ring.

11. The composition as claimed in claim 10, wherein:
L⁵, L⁶, and L^{b} are each independently a single bond, a substituted or unsubstituted phenylene group, a substituted or unsubstituted biphenylene group, or a substituted or unsubstituted terphenylene group, and
Ar² and Ar³ are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, or a substituted or unsubstituted fluorenyl group.

12. An organic optoelectronic device, comprising:
an anode and a cathode facing each other, and
an organic layer between the anode and the cathode, the organic layer including the compound as claimed in claim 1.

13. The organic optoelectronic device as claimed in claim 12, wherein:
the organic layer includes a light emitting layer, and
the light emitting layer includes a fluorescent dopant, a phosphorescent dopant, or a combination thereof and the compound.

14. A display device comprising the organic optoelectronic device as claimed in claim 12.

15. An organic optoelectronic device, comprising:
an anode and a cathode facing each other, and
an organic layer between the anode and the cathode, the organic layer including the composition as claimed in claim 4.

16. The organic optoelectronic device as claimed in claim 15, wherein:
the organic layer includes a light emitting layer, and
the light emitting layer includes a fluorescent dopant, a phosphorescent dopant, or a combination thereof and the composition.

17. A display device comprising the organic optoelectronic device as claimed in claim 15.
